(19) United States
(12) Patent Application Publication
(10) Pub. No.: US 2003/0124176 A1
(43) Pub. Date: Jul. 3, 2003

(54) Transdermal and topical administration of drugs using basic permeation enhancers

(76) Inventors: Tsung-Min Hsu, San Diego, CA (US); Nicole T. Gricenko, San Diego, CA (US); Alan T.J. Hickey, San Diego, CA (US); Eric C. Jacobson, San Diego, CA (US); Rose C. LoBello, San Diego, CA (US); Jane Obara, San Diego, CA (US); Eric C. Luo, Plano, TX (US)

Correspondence Address: REED & ASSOCIATES, 800 MENLO AVENUE, SUITE 210, MENLO PARK, CA, 94025, US

(21) Appl. No.: 10/176,952

(22) Filed: Jun. 21, 2002

Related U.S. Application Data
(63) Continuation-in-part of application No. 09/972,008, filed on Oct. 4, 2001.
Application No. 09/972,008 is a continuation-in-part of application No. 09/738,410, filed on Dec. 14, 2000.
Application No. 09/738,410 is a continuation-in-part of application No. 09/569,889, filed on May 11, 2000.
Application No. 09/569,889 is a continuation-in-part of application No. 09/465,098, filed on Dec. 16, 1999, now abandoned.
Application No. 09/569,889 is a continuation-in-part of application No. 09/738,395, filed on Dec. 14, 2000.
Application No. 09/738,395 is a continuation of application No. 09/607,892, filed on Jun. 30, 2000, now abandoned.

(51) Int. Cl. A61K 9/70

(52) U.S. Cl. 424/449

(57) ABSTRACT

Methods are provided for enhancing the permeability of skin or mucosal tissue to topical or transdermal application of pharmacologically or cosmeceutically active agents. The methods entail the use of a base in order to increase the flux of the active agent through a body surface while minimizing the likelihood of skin damage, irritation or sensitization. The permeation enhancer can be an inorganic or organic base. Compositions and transdermal systems are also described.

### CROSS-REFERENCE TO RELATED APPLICATIONS

This application is a continuation in part of U.S. Ser. No. 09/972,008 filed on Oct. 4, 2001, which is a continuation in part of U.S. Ser. No. 09/738,410 filed on Dec. 14, 2000, which is a continuation in part of U.S. Ser. No. 09/569,889 filed on May 11, 2000, which is a continuation in part of U.S. Ser. No. 09/465,098 filed on Dec. 16, 1999; and is a continuation in part of U.S. Ser. No. 09/738,395 filed on Dec. 14, 2000, which is a continuation in part of U.S. Ser. No. 09/607,892 filed Jun. 30, 2000, now abandoned.

### FIELD OF THE INVENTION

This invention relates generally to the topical and transdermal administration of pharmacologically or cosmeceutically active agents, and more particularly relates to methods and compositions for enhancing the flux of pharmacologically active agents through a body surface by treatment with a basic permeation enhancer.

### BACKGROUND OF THE INVENTION

The delivery of drugs through the skin provides many advantages; primarily, such a means of delivery is a comfortable, convenient and noninvasive way of administering drugs. The variable rates of absorption and metabolism encountered in oral treatment are avoided, and other inherent inconveniences, e.g., gastrointestinal irritation and the like, are eliminated as well. Transdermal drug delivery also makes possible a high degree of control over blood concentrations of any particular drug.

Skin is a structurally complex, relatively thick membrane. Molecules moving from the environment into and through intact skin must first penetrate the stratum corneum and any material on its surface. They must then penetrate the viable epidermis, the papillary dermis, and the capillary walls into the blood stream or lymph channels. To be so absorbed, molecules must overcome a different resistance to penetration in each type of tissue. Transport across the skin membrane is thus a complex phenomenon. However, it is the cells of the stratum corneum, which present the primary barrier to absorption of topical compositions or transdermally administered drugs. The stratum corneum is a thin layer of dense, highly keratinized cells approximately 10-15 microns thick over most of the body. It is believed to be the high degree of keratinization within these cells as well as their dense packing which creates in most cases a substantially impermeable barrier to drug penetration. With many drugs, the rate of permeation through the skin is extremely low without the use of some means to enhance the permeability of the skin.

Numerous chemical agents have been studied as a means of increasing the rate at which a drug penetrates through the skin. As will be appreciated by those in the field, chemical enhancers are compounds that are administered along with the drug (or in some cases the skin may be pretreated with a chemical enhancer) in order to increase the permeability of the stratum corneum, and thereby provide for enhanced penetration of the drug through the skin. Ideally, such chemical penetration enhancers or “permeation enhancers,” as the compounds are referred to herein, are compounds that are innocuous and serve merely to facilitate diffusion of the drug through the stratum corneum. The permeability of many therapeutic agents with diverse physicochemical characteristics may be enhanced using these chemical enhancement means. However, there are skin irritation and sensitization problems associated with high levels of certain enhancers.

Accordingly, although there are many chemical methods of enhancing permeation, there remains an ongoing need for a method that is highly effective in increasing the rate at which a drug permeates the skin, does not result in skin damage, irritation, sensitization, or the like, and can be used to effect transdermal delivery of even high molecular weight drugs such as peptides, proteins, and nucleic acids. It has now been discovered that basic permeation enhancers as described herein are highly effective permeation enhancers, and provide all of the aforementioned advantages relative to known permeation enhancers. Furthermore, in contrast to many conventional enhancers, transdermal administration of drugs with basic permeation enhancers, employed at the appropriate levels, does not result in systemic toxicity.

### SUMMARY OF THE INVENTION

One aspect of the invention pertains to a method for enhancing the flux of a drug through a body surface, comprising: (a) administering the drug to a localized region of a human patient's body surface; and (b) administering a basic permeation enhancer to the localized region, the enhancer comprising a pharmaceutically acceptable base and being present in an amount effective to provide a pH within the range of about 8.0-13.0 at the localized region of the body surface during administration of the drug and to enhance the flux of the drug through the body surface without causing damage thereto. In one aspect of the invention the pH is about 8.5-11.5, in another aspect the pH is about 9.5-11.5, and most preferably about 10.0 to 11.5. The pharmaceutically acceptable base can be an inorganic or an organic base.

Another aspect of the invention relates to a composition for the enhanced delivery of a drug through a body surface, comprising a formulation of: (a) a therapeutically effective amount of the drug; (b) a pharmaceutically acceptable base, in an amount effective to provide a pH within the range of about 8.0-13.0 at the body surface during administration of the drug and to enhance the flux of the drug through the body surface without causing damage thereto; and (c) a pharmaceutically acceptable carrier suitable for topical or transdermal drug administration. In one aspect of the invention the pH is about 8.5-11.5, in another aspect the pH is about 9.5-11.5, and most preferably about 10.0 to 11.5. The formulation is typically aqueous. The pharmaceutically acceptable base can be an inorganic or an organic base.

Yet another aspect of the invention pertains to a system for the enhanced topical or transdermal administration of a drug, comprising: (a) at least one drug reservoir containing the drug and a pharmaceutically acceptable base, in an amount effective to enhance the flux of the drug through the body surface without causing damage thereto; (b) a means for maintaining the system in drug and base transmitting relationship to the body surface and forming a body surface-system interface; and (c) a backing layer that serves as the outer surface of the device during use, wherein the base is effective to provide a pH within the range of about 8.0-13.0 at the body surface-system interface during administration of the drug. In one aspect of the invention the pH is about 8.5-11.5, in another aspect the pH is about 9.5-11.5, and most preferably about 10.0 to 11.5. The pharmaceutically acceptable base can be an inorganic or an organic base.

### DETAILED DESCRIPTION OF THE INVENTION

The present invention provides a method for enhancing the flux of an active agent through a body surface. The active agent and a basic permeation enhancer are administered to a localized region of a human patient's body surface. The permeation enhancer is a pharmaceutically acceptable base, and is present in an amount effective to: a) provide a pH within the range of about 8.0-13.0 at the localized region of the body surface during administration of the drug and b) enhance the flux of the active agent through the body surface without causing damage thereto. Examples of suitable permeation enhancers are described below. The active agent and permeation enhancer may be present in a single pharmaceutical formulation, or they may be in separate pharmaceutical formulations.

The steps of (a) administering the active agent and (b) administering the basic permeation enhancer can be done in any order. For example, step (a) can be done prior to step (b); step (b) can be done prior to step (a); and steps (a) and (b) can be done simultaneously. Certain methods may be preferred depending upon the selection of active agent and basic permeation enhancer, as well as taking into consideration ease of patient compliance and so forth. For example, performing steps (a) and (b) simultaneously, is one preferred method of the invention.

I. Definitions and Nomenclature

Before describing the present invention in detail, it is to be understood that this invention is not limited to particular drugs or drug delivery systems, as such may vary. It is also to be understood that the terminology used herein is for the purpose of describing particular embodiments only, and is not intended to be limiting. In addition, before describing detailed embodiments of the invention, it will be useful to set forth definitions that are used in describing the invention. The definitions set forth apply only to the terms as they are used in this patent and may not be applicable to the same terms as used elsewhere, for example in scientific literature or other patents or applications including other applications by these inventors or assigned to common owners. Additionally, when examples are given, they are intended to be exemplary only and not to be restrictive.

It must be noted that, as used in this specification and the appended claims, the singular forms “a,” “an” and “the” include plural referents unless the context clearly dictates otherwise. Thus, for example, reference to “a pharmacologically active agent” includes a mixture of two or more such compounds, reference to “a base” includes mixtures of two or more bases, and the like.

In describing and claiming the present invention, the following terminology will be used in accordance with the definitions set out below.

“Active agent,” “pharmacologically active agent” and “drug” are used interchangeably herein to refer to a chemical material or compound that induces a desired pharmacological, physiological effect, and include agents that are therapeutically effective, prophylactically effective, or cosmeceutically effective. The terms also encompass pharmaceutically acceptable, pharmacologically active derivatives and analogs of those active agents specifically mentioned herein, including, but not limited to, salts, esters, amides, prodrugs, active metabolites, inclusion complexes, analogs, and the like. When the terms “active agent,” “pharmacologically active agent” and “drug” are used, then, it is to be understood that applicants intend to include the active agent per se as well as pharmaceutically acceptable, pharmacologically active salts, esters, amides, prodrugs, active metabolites, inclusion complexes, analogs, etc., which are collectively referred to herein as “pharmaceutically acceptable derivatives”. The term “active agent” is also intended to encompass “cosmeceutically active agents”, which are nontoxic agents that have medicinal or drug-like properties which, when applied to the surface of skin, beneficially affect the biological functioning of that skin.

The term “aqueous” refers to a composition, formulation or drug delivery system that contains water or that becomes water-containing following application to the skin or mucosal tissue.

The term “base” is used in its traditional sense, i.e., a substance that dissolves in water to produce hydroxide ions. The water is typically an aqueous fluid, and may be natural moisture at the skin surface, or the patch or composition that is used may contain added water, and/or be used in connection with an occlusive backing. Similarly, any liquid or semisolid formulation that is used is preferably aqueous or used in conjunction with an overlayer of an occlusive material. Any base may be used provided that the compound provides free hydroxide ions in the presence of an aqueous fluid. Bases can provide free hydroxide ions either directly or indirectly and thus can also be referred to as “hydroxide-releasing agents”. Hydroxide-releasing agents that provide free hydroxide ions directly, typically contain hydroxide groups and release the hydroxide ions directly into solution, for example, alkali metal hydroxides. Hydroxide-releasing agents that provide free hydroxide ions indirectly, are typically those compounds that are acted upon chemically in an aqueous environment and the reaction produces hydroxide ions, for example metal carbonates or amines.

“Body surface” is used to refer to skin or mucosal tissue.

“Carriers” or “vehicles” as used herein refer to carrier materials suitable for transdermal or topical drug administration. Carriers and vehicles useful herein include any such materials known in the art, which are nontoxic and do not interact with other components of the composition in a deleterious manner.

“Effective amount” or “a cosmeceutically effective amount” of a cosmeceutically active agent is meant a nontoxic but sufficient amount of a cosmeceutically active agent to provide the desired cosmetic effect.

“Effective amount” or “a therapeutically effective amount” of a therapeutically active agent is intended to mean a nontoxic but sufficient amount of a therapeutically active agent to provide the desired therapeutic effect. The amount that is effective will vary from subject to subject, depending on the age and general condition of the individual, the particular active agent or agents, and the like. Thus, it is not always possible to specify an exact effective amount. However, an appropriate effective amount in any individual case may be determined by one of ordinary skill in the art using routine experimentation. Furthermore, the exact effective amount of an active agent incorporated into a composition or dosage form of the invention is not critical, so long as the concentration is within a range sufficient to permit ready application of the formulation so as to deliver an amount of the active agent that is within a therapeutically effective range.

“Effective amount” or “an effective permeation enhancing amount” of a permeation enhancer refers to a nontoxic, non-damaging but sufficient amount of the enhancer composition to provide the desired increase in skin permeability and, correspondingly, the desired depth of penetration, rate of administration, and amount of drug delivered.

“Penetration enhancement” or “permeation enhancement” as used herein relates to an increase in the permeability of the skin or mucosal tissue to the selected pharmacologically active agent, i.e., so that the rate at which the agent permeates therethrough (i.e., the “flux” of the agent through the body surface) is increased relative to the rate that would be obtained in the absence of permeation enhancer. The enhanced permeation effected through the use of such enhancers can be observed by measuring the rate of diffusion of drug through animal or human skin using, for example a Franz diffusion apparatus as known in the art and as employed in the Examples herein.

“Predetermined area” of skin or mucosal tissue refers to the area of skin or mucosal tissue through which a drug-enhancer formulation is delivered, and is a defined area of intact unbroken living skin or mucosal tissue. That area will usually be in the range of about 5-200 cm², more usually in the range of about 5-100 cm², preferably in the range of about 20-60 cm². However, it will be appreciated by those skilled in the art of drug delivery that the area of skin or mucosal tissue through which drug is administered may vary significantly, depending on patch configuration, dose, and the like.

“Topical administration” is used in its conventional sense to mean delivery of a topical drug or pharmacologically active agent to the skin or mucosa, as in, for example, the treatment of various skin disorders. Topical administration, in contrast to transdermal administration, provides a local rather than a systemic effect. However, unless otherwise stated or implied, the terms “topical drug administration” and “transdermal drug administration” are used interchangeably.

“Transdermal” drug delivery is meant administration of a drug to the skin surface of an individual so that the drug passes through the skin tissue and into the individual's blood stream, thereby providing a systemic effect. The term “transdermal” is intended to include “transmucosal” drug administration, i.e., administration of a drug to the mucosal (e.g., sublingual, buccal, vaginal, rectal) surface of an individual so that the drug passes through the mucosal tissue and into the individual's blood stream.

“Treating” and “treatment” as used herein refer to reduction in severity and/or frequency of symptoms, elimination of symptoms and/or underlying cause, prevention of the occurrence of symptoms and/or their underlying cause, and improvement or remediation of damage. The present method of “treating” a patient, as the term is used herein, thus encompasses both prevention of a disorder in a predisposed individual and treatment of the disorder in a clinically symptomatic individual.

II. The Permeation Enhancers

The permeation enhancer of the invention is an inorganic or an organic pharmaceutically acceptable base. Preferred inorganic bases include inorganic hydroxides, inorganic oxides, inorganic salts of weak acids, and combinations thereof. Preferred organic bases are nitrogenous bases.

It has long been thought that strong bases, such as NaOH, were not suitable as permeation enhancers because they would damage skin. It has been now been discovered that the skin permeability of various drugs could be enhanced without skin damage by exposing the skin to a base or basic solution, in a skin contacting formulation or patch. The desired pH of the solution on the skin can be obtained using a variety of bases or base concentrations. Accordingly, the pH is selected so as to be low enough so as to not cause skin damage, but high enough to enhance skin permeation to various active agents. As such, it is important that the amount of base in any patch or formulation is optimized so as to increase the flux of the drug through the body surface while minimizing any possibility of skin damage. In general, this means that the pH at the body surface in contact with a formulation or drug delivery system of the invention (i.e., the interface between the body surface and the formulation or delivery system) is preferably in the range of approximately 8.0-13.0, preferably about 8.5-11.5, more preferably about 9.5-11.5 and most preferably about 10.0 to 11.5.

In one preferred embodiment, the pH at the interface is the primary design consideration, i.e., the composition or system is designed so as to provide the desired pH at the interface. Anhydrous formulations and transdermal systems may not have a measurable pH, and the formulation or system can be designed so as to provide a target pH at the interface. Moisture from the body surface can migrate into the formulation or system, dissolve the base and thus release the base into solution, which will then provide the desired target pH at the interface. In those instances, a hydrophilic composition is preferred. In addition, when using aqueous formulations, the pH of the formulation may change over time after it is applied on the skin. For example, gels, solutions, ointments, etc., may experience a net loss of moisture after being applied to the body surface, i.e., the amount of water lost is greater than the amount of water received from the body surface. In that case, the pH of the formulation may be different than its pH when manufactured. This problem can be easily remedied by designing the aqueous formulations to provide a target pH at the interface.

In other embodiments of the invention, the pH of the formulation or the drug composition contained within a delivery system will be in the range of approximately 8.0-13.0, preferably about 8.5-11.5, more preferably about 9.5-11.5 and most preferably about 10.0 to 11.5.

In one embodiment of the invention the pH of the formulation is higher than the pH at the interface. For example, if an aqueous formulation is used, moisture from the body surface can dilute the formulation, and thus provide for a different pH at the interface, which will typically be lower than that of the formulation itself.

In one preferred embodiment, the body surface is exposed to a base or basic solution for a sufficient period of time so as to provide a high pH at the skin surface, thus creating channels in the skin or mucosa for the drug to go through. It is expected that drug flux is proportional to the strength of the solution and the duration of exposure. However, it is desirable to balance the maximization of drug flux with the minimization of skin damage. This can be done in numerous ways. For example, the skin damage may be minimized by selecting a lower pH within the 8.0-13.0 range, by exposing the skin to the formulation or system for a shorter period of time, or by including at least one irritation-mitigating additive. Alternatively, the patient can be advised to change the location of application with each subsequent administration.

While certain preferred amounts are set forth below, it is understood that, for all of the inorganic and organic bases described herein, the optimum amount of any such base will depend on the strength or weakness of the base and its molecular weight, and other factors such as the number of ionizable sites in the active agent being administered and whether there are any acidic species present in the formulation or patch. One skilled in the art may readily determine the optimum amount for any particular base such that the degree of enhancement is optimized while the possibility of damage to the body surface is eliminated or at least substantially minimized.

A. Inorganic Base

Exemplary inorganic bases are inorganic hydroxides, inorganic oxides, inorganic salts of weak acids, and combinations thereof. Preferred inorganic bases are those whose aqueous solutions have a high pH, and are acceptable as food or pharmaceutical additives. Examples of such preferred inorganic bases are those listed below, along with their respective pHs. Some of the bases are identified by their hydrate forms, and it is understood that when referring to a “base”, both the hydrated and non-hydrated forms are intended to be included.

| | pH of Aqueous Solution |
|---|---|
| Inorganic base | (concentration) |
| Ammonium hydroxide^{1,2,3} | 11.27 (1 N), 10.27 (0.001 N) |
| Sodium hydroxide^{1,2,3} | 14 (5%), 13 (0.5%), 12 (0.05%) |
| Potassium hydroxide^{1,2,3} | 13.5 (0.1 M) |
| Calcium hydroxide^{1,3} | 12.4 (saturated solution in water) |
| Magnesium hydroxide^{1,3} | 9.5 to 10.5 slurry |
| Magnesium oxide^{1,2,3} | 10.3 (saturated aqueous solution) |
| Calcium oxide³ | Soluble in water, Form Ca(OH)₂ |
| Sodium acetate^{1,3} | ˜8.9 (0.1 N) |
| Sodium acetate, trihydrate^{1,2} | 8.9 (0.1 N) |
| Sodium acetate, anhydrous^{1,2} | ˜8.9 (0.1 N) |
| Sodium borate decahydrate^{1,2} | ˜8.8-9.4, 9.15 to 9.2 (0.01 M) |
| Sodium borate^{1,2,3} | 8.8-9.4, 9.15 to 9.2 (0.01 M) |
| Sodium metaborate | Strongly alkaline |
| Sodium carbonate^{1,2,3} | ˜11.6 |
| Sodium carbonate hydrate¹ | ˜11.6 |
| Sodium carbonate anhydrous | ˜11.6 |
| Sodium bicarbonate^{1,2,3} | 8.3 (0.1 M fresh) |
| Sodium phosphate, tribasic^{1,3} | ˜11.5 (0.1%), ˜11.7 (0.5%), |
| | ˜11.9 (1.0%) |
| Sodium phosphate, tribasic | 11.5 (0.1%), 11.7 (0.5%), |
| dodecahydrate | 11.9 (1.0%) |
| Sodium phosphate, dibasic, | 9.1 (1%) |
| anhydrous^{1,2} | |
| Sodium phosphate, dibasic, | ˜9.5 |
| heptahydrate^{1,2} | |
| Sodium phosphate, dibasic^{1,3} | ˜9.5 |
| Sodium phosphate, dibasic, | ˜9.5 |
| dihydrate¹ | |
| Sodium phosphate, dibasic, | ˜9.5 |
| dodecahydrate | |
| Potassium carbonate^{1,3} | ˜11.6 |
| Potassium bicarbonate³ | 8.2 (0.1 M) |
| Potassium citrate^{1,2,3} | ˜8.5 |
| Potassium citrate monohydrate | ˜8.5 |
| Potassium acetate^{1,3} | 9.7 (0.1 M) |
| Potassium phosphate, dibasic^{1,2} | Aqueous solution is slightly |
| | alkaline |
| Potassium phosphate, tribasic³ | Aqueous solution is strongly |
| | alkaline |
| Ammonium phosphate, dibasic^{1,2,3} | ˜8 |

¹listed in the “Chemicals in Compliance with Pharmaceutical Standards: Inactive Ingredient Guide”
²listed in the “Handbook of Pharmaceutical Additives”
³listed in the FDA's food additive database

### Inorganic Hydroxides

Inorganic hydroxides include, for example, ammonium hydroxide, alkali metal hydroxide and alkaline earth metal hydroxides, and mixtures thereof. Preferred inorganic hydroxides include ammonium hydroxide; monovalent alkali metal hydroxides such as sodium hydroxide and potassium hydroxide; divalent alkali earth metal hydroxides such as calcium hydroxide and magnesium hydroxide; and combinations thereof.

The amount of inorganic hydroxide included in the compositions and systems of the invention, will typically represent about 0.3-7.0 wt %, preferably 0.5-4.0 wt %, more preferably about 0.5-3.0 wt %, most preferably about 0.75-2.0 wt %, of a topically applied formulation or of a drug reservoir of a drug delivery system, or patch.

The aforementioned amounts are particularly applicable to those formulations and patches in which the active agent is (1) an uncharged molecule, e.g., wherein a basic drug is in nonionized, free-base form, (2) a basic salt of an acidic drug, or (3) there are no additional species in the formulation or patch that could react with or be neutralized by the inorganic hydroxide, to any significant degree.

For formulations and patches in which the drug is in the form of an acid addition salt, and/or wherein there are additional species in the formulations or systems that can be neutralized by or react with the inorganic base (i.e., acidic inactive ingredients), the amount of inorganic hydroxide is preferably the total of (1) the amount necessary to neutralize the acid addition salt and/or other base-neutralizable species (i.e., the “acidic species”), plus (2) about 0.3-7.0 wt %, preferably 0.5-4.0 wt %, more preferably about 0.5-3.0 wt %, most preferably about 0.75-2.0 wt %, of the formulation or drug reservoir. That is, for an acid addition salt, the enhancer is preferably present in an amount just sufficient to neutralize the salt, plus an additional amount (i.e., about 0.3-7.0 wt %, preferably 0.5-4.0 wt %, more preferably about 0.5-3.0 wt %, most preferably about 0.75-2.0 wt %) to enhance the flux of the drug through the skin or mucosal tissue. Basic drugs in the form of a neutral, free base or basic salt of acidic drug are usually not affected by a base, and thus for these drugs, the amount in (1) is usually the amount necessary to neutralize inactive components that are acidic. For patches, the aforementioned percentages are given relative to the total weight of the formulation components and the adhesive, gel or liquid reservoir.

Still greater amounts of inorganic hydroxide may be used by controlling the rate and/or quantity of release of the base, preferably during the drug delivery period itself.

### Inorganic Oxides

Inorganic oxides include, for example, magnesium oxide, calcium oxide, and the like.

The amount of inorganic oxide included in the compositions and systems of the invention may be substantially higher than the numbers set forth above for the inorganic hydroxide, and may be as high as 20 wt %, in some cases as high as 25 wt % or higher, but will generally be in the range of about 2-20 wt %. These amounts may be adjusted to take into consideration the presence of any base-neutralizable species.

### Inorganic Salts of Weak Acids

Inorganic salts of weak acids include, ammonium phosphate (dibasic); alkali metal salts of weak acids such as sodium acetate, sodium borate, sodium metaborate, sodium carbonate, sodium bicarbonate, sodium phosphate (tribasic), sodium phosphate (dibasic), potassium carbonate, potassium bicarbonate, potassium citrate, potassium acetate, potassium phosphate (dibasic), potassium phosphate (tribasic); alkaline earth metal salts of weak acids such as magnesium phosphate and calcium phosphate; and the like, and combinations thereof.

Preferred inorganic salts of weak acids include, ammonium phosphate (dibasic) and alkali metal salts of weak acids.

The amount of inorganic salts of weak acids included in the compositions and systems of the invention may be substantially higher than the numbers set forth above for the inorganic hydroxide, and may be as high as 20 wt %, in some cases as high as 25 wt % or higher, but will generally be in the range of approximately 2-20 wt %. These amounts may be adjusted to take into consideration the presence of any base-neutralizable species.

B. Organic Bases

Organic bases suitable for use in the invention are compounds having an amino group, amido group, an oxime, a cyano group, an aromatic or non-aromatic nitrogen-containing heterocycle, a urea group, and combinations thereof. More specifically, examples of suitable organic bases are nitrogenous bases, which include, but are not limited to, primary amines, secondary amines, tertiary amines, amides, oximes, cyano (—CN) containing groups, aromatic and non-aromatic nitrogen-containing heterocycles, urea, and mixtures thereof. Preferred organic bases are primary amines, secondary amines, tertiary amines, aromatic and non-aromatic nitrogen-containing heterocycles, and mixtures thereof.

For nitrogenous bases, the amount of enhancing agent will typically represent about 0.5-4.0 wt %, preferably about 0.5-3.0 wt %, more preferably about 0.75-2.0 wt %, of a topically applied formulation or of a drug reservoir of a drug delivery system or a patch. These amounts may be adjusted to take into consideration the presence of any base-neutralizable species.

Still greater amounts of the nitrogenous base may be used depending on the strength of the base and the rate and/or quantity of release of the nitrogenous base preferably during the drug delivery period itself.

Preferred organic bases are those whose aqueous solutions have a high pH or a high pKa (more preferably a pKa>9), and are acceptable as food or pharmaceutical additives. Examples of such preferred organic bases are those listed below, along with their respective pHs (or pKa values).

| | pH of Aqueous Solution |
|---|---|
| Organic base | (concentration) |
| 2-amino-2-methyl-1,3-propanediol¹ | 10.8 (0.1 m) |
| 2-amino-2-methyl-1-propanol¹ | 11.3 (0.1 m) |
| Diethanolamine¹ | 11.0 (0.1 N) |
| Triethanolamine¹ | 10.5 (0.1 N) |
| Butylamine² | pKa = 10.56 |
| Dimethylamine² | Strong base, pKa = 10.73 |
| Cyclohexylamine² | Strong base, pKa = 10.64 |
| Ethylenediamine² | Strong base, pKa = 10.71 |
| Isopentylamine² | pKa = 10.6 |
| Monoethanolamine² | 12.1 (25%), 12.05 (0.1 N), pKa = 9.4 |
| Phenethylamine² | Strong base, pKa = 9.83 |
| Piperidine² | Strong base, pKa = 11.12 |
| Pyrrolidine² | Strong base, pKa = 11.27 |
| Trimethylamine² | Strong base, pKa = 9.81 |

¹listed in the “Handbook of Pharmaceutical Additives”
²listed in the FDA's food additive database

### Amines

Amines are compounds that include at least one primary amino (—NH₂) group, mono-substituted (secondary) amino group or di-substituted (tertiary) amino group.

Primary amino groups, secondary amino groups, and tertiary amino groups may be generically grouped as encompassed by the molecular structure —NR¹R²R³ wherein R¹, R², and R³ may be the same or different and are generally selected from the group consisting of H, alkyl, hydroxyalkyl, alkoxyalkyl, alkenyl, hydroxyalkenyl, alkoxyalkenyl, cycloalkyl, cycloalkyl-substituted alkyl, monocyclic aryl, and monocyclic aryl-substituted alkyl, all of which may be substituted with one or more nonhydrocarbyl substituents, e.g., 1 to 3 halo, hydroxyl, thiol, or lower alkoxy groups.

Exemplary primary amines include 2-aminoethanol, 2-aminoheptane, 2-amino-2methyl-1,3 propanediol, 2-amino-2-methyl-1-propanol, n-amylamine, benzylamine, 1,4-butanediamine, n-butylamine, cyclohexylamine, ethylamine, ethylenediamine, methylamine, α-methylbenzylamine, phenethylamine, propylamine, and tris(hydroxymethyl)aminomethane.

Exemplary secondary amines include compounds that contain groups such as methylamino, ethylamino, isopropylamino, butylamino, cyclopropylamino, cyclohexylamino, n-hexylamino, phenylamino, benzylamino, chloroethylamino, hydroxyethylamino, and so forth. Exemplary secondary amines include diethanolamine, diethylamine, diisopropylamine, and lamine.

Exemplary tertiary amines include compounds that contain groups such as dibutylamino, diethylamino, dimethylamino, diisopropylamino, ethylchloroethylamino, ethylcyclopropylamino, methylhexylamino, methylcyclohexylamino, methylpropylamino, methylbenzylamino, methyl-p-chlorophenylamino, methylcyclohexylamino, methylphenylamino, methyltoluylamino, and so forth. Exemplary tertiary amines include N,N-diethylaniline, N,N-dimethylglycine, triethanolamine, triethylamine, and trimethylamine.

### Amides

Amides are compounds that include an amido group that has the molecular structure —(CO)—NR¹R² where R¹ and R² can be the same or different, and are generally selected from the groups consisting of H, alkyl, hydroxyalkyl, alkoxyalkyl, alkenyl, hydroxyalkenyl, alkoxyalkenyl, cycloalkyl, cycloalkyl-substituted alkyl, monocyclic aryl, and monocyclic aryl-substituted alkyl, all of which may be substituted with one or more nonhydrocarbyl substituents, e.g., 1 to 3 halo, hydroxyl, thiol, or lower alkoxy groups.

### Aromatic Nitrogen-Containing Heterocycles

Aromatic nitrogen-containing heterocycles, typically contain a 5- or 6-membered monocyclic substituent, or a bicyclic fused or linked 5- or 6-membered ring, such as imidazolyl, indolyl, pyridinyl, pyrimidinyl, pyrrolyl, quinolinyl, tetrazolyl, 1,2,4-triazolyl, etc.

Aromatic nitrogen-containing heterocycles suitable as the organic base herein include, by way of example, 2-amino-pyridine, benzimidazole, 2,5-diaminopyridine, 2,4-dimethylimidazole, 2,3-dimethylpyridine, 2,4-dimethylpyridine, 3,5-dimethylpyridine, imidazole, methoxypyridine, γ-picoline, 2,4,6-trimethylpyridine, and combinations thereof.

### Non-Aromatic Nitrogen-Containing Heterocycles

Non-aromatic nitrogen-containing heterocycles, typically contain 4- to 6-membered rings such as acetimido, morpholinyl, lactams and imides (e.g., γ-butyrolactam, ε-caprolactam, N-phenyl-β-propiolactam), phthalimido, piperidyl, piperidino, piperazinyl, pyrrolidinyl, succinimido, etc.

Non-aromatic nitrogen-containing heterocycles include, by way of example, 1,2-dimethylpiperidine, 2,5-dimethylpiperazine, 1,2-dimethylpyrrolidine, 1-ethylpiperidine, n-methylpyrrolidine, morpholine, piperazine, piperidine, pyrrolidine, 2,2,6,6-tetramethylpiperidine, 2,2,4-trimethylpiperidine, and combinations thereof.

III. The Active Agent

The active agent administered may be any compound that is suitable for topical, transdermal or transmucosal delivery and induces a desired local or systemic effect. Such substances include the broad classes of compounds normally delivered through body surfaces and membranes, including skin. While appreciating the fact that active agents may be classified in more than one category, exemplary categories of interest include: Alzheimer's drugs; analgesic agents such as narcotic analgesics; anesthetic agents; anti-acne agents; anti-anxiety drugs; anti-arthritic agents; anti-arrhythmic agents; anti-asthmatic agents and other respiratory drugs; antibiotics including antibacterial agents; anticancer agents, including antineoplastic drugs; anticholinergics and anticholinergic antagonists; anticonvulsants; antidepressants; antidiabetic agents; antidiarrheals; anti-emetics; antifungal agents; antiglaucoma agents; antihelminthics; antihistamines; antihyperlipidemic agents; antihypertensive agents; anti-infective agents such as antibiotics and antiviral agents; anti-inflammatory agents; antilipemic agents; antimigraine preparations; antinauseants; antineoplastic agents; antipanic agents; antiparkinsonism drugs; antipruritics; antipsoriatics; antipsychotics; antipyretics; antirheumatic agents; antispasmodics; antitubercular agents; antitussive agents; anti-ulcer agents; antiviral agents; anxiolytics; appetite stimulants and suppressants; attention deficit disorder (ADD) and attention deficit hyperactivity disorder (ADHD) drugs; benign prostatic hyperplasia agents; beta-blockers and anti-arrhythmic agents; bone density regulators; cardiovascular preparations including calcium channel blockers; central nervous system agents; central nervous system stimulants; cholesterol-lowering agents; cough and cold preparations, including decongestants; depigmenting agents; diuretics; erectile dysfunction therapies; fatty acids; gastrointestinal agents; genetic materials; hematinic agents; hemostatic drugs; herbal remedies; hormonolytics; hypnotics; hypocalcemics; hypoglycemic agents; immunosuppressive agents; leukotriene inhibitors; mitotic inhibitors; muscle relaxants; narcotic antagonists; nicotine; nutritional agents, such as vitamins, minerals, essential amino acids and fatty acids; motion sickness drugs; oxytocics; parasympatholytics; peptide drugs; prostaglandins; psychostimulants; sedatives; serotonin antagonists; serotonin receptor agonists and antagonists; steroids; sympathomimetics; thyroid preparations; tocolytics; topoimerase inhibitors; Tourette's Syndrome agents; tranquilizers; vasodilators including general coronary, peripheral and cerebral; wart preparations; and combinations thereof.

The active agent administered also may be one that is cosmetically or “cosmeceutically” effective rather than pharmacologically active. Such agents include, for example, compounds that can reduce the appearance of aging or photodamaged skin, e.g., alpha hydroxyacids, alpha ketoacids, polymeric hydroxyacids, moisturizers, collagen, marine extract, and antioxidants such as ascorbic acid (vitamin C), α-tocopherol (Vitamin E), β-tocopherol, γ-tocopherol, δ-tocopherol, ε-tocopherol, ζ₁-tocopherol, ζ₂-tocopherol, η-tocopherol, and retinol (vitamin A), and/or cosmetically acceptable salts, esters, amides, or other derivatives thereof. A preferred tocopherol compound is α-tocopherol. Additional cosmetic agents include those that are capable of improving oxygen supply in skin tissue, as described, for example, in Gross, et al, WO 94/00098 and Gross, et al, WO 94/00109, both assigned to Lancaster Group AG. Sunscreens may also be included.

The active agent may be administered, if desired, in the form of a salt, ester, amide, prodrug, derivative, or the like, provided the salt, ester, amide, prodrug or derivative is suitable pharmacologically. Salts, esters, amides, prodrugs and other derivatives of the active agents may be prepared using standard procedures known to those skilled in the art of synthetic organic chemistry and described, for example, by March's Advanced Organic Chemistry: Reactions, Mechanisms and Structure, 5th Ed. (Wiley-Interscience, 2001).

For example, acid addition salts are prepared from the free base (e.g., an amine drug) using conventional methodology, by reaction with a suitable acid. Generally, the base form of the drug is dissolved in a polar organic solvent such as methanol or ethanol and the acid is added thereto. The resulting salt either precipitates or is brought out of solution by the addition of a less polar solvent. Suitable acids for preparing acid addition salts include both organic acids (e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like), as well as inorganic acids (e.g., hydrochloric acid, hydrobromic acid, sulfuric acid, nitric acid, phosphoric acid, and the like). An acid addition salt may be reconverted to the free base by treatment with a suitable base. Particularly preferred acid addition salts of the active agents herein are halide salts, such as may be prepared using hydrochloric or hydrobromic acids.

Preparation of basic salts of acids are prepared in a similar manner using a pharmaceutically acceptable base such as sodium hydroxide, potassium hydroxide, ammonium hydroxide, calcium hydroxide, trimethylamine, or the like. Particularly preferred basic salts herein are alkali metal salts, e.g., the sodium salt, and copper salts.

Preparation of esters involves functionalization of hydroxyl and/or carboxyl groups that may be present within the molecular structure of the drug. The esters are typically acyl-substituted derivatives of free alcohol groups, i.e., moieties that are derived from carboxylic acids of the formula RCOOH where R is alkyl, and preferably is lower alkyl. Esters can be reconverted to the free acids, if desired, by using conventional hydrogenolysis or hydrolysis procedures. Amides and prodrugs may also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety, which results in a compound that is therapeutically inactive until modified by an individual's metabolic system.

For those active agents that are chiral in nature and can thus be in an enantiomerically pure form or in a racemic mixture, the drug may be incorporated into the formulation either as the racemate or in the enantiomerically pure form.

The amount of active agent administered will depend on a number of factors and will vary from subject to subject and depend on the particular drug administered, the particular disorder or condition being treated, the severity of the symptoms, the subject's age, weight and general condition, and the judgment of the prescribing physician. Other factors, specific to transdermal drug delivery, include the solubility and permeability of the carrier and adhesive layer in a drug delivery device, if one is used, and the period of time for which such a system will be fixed to the skin or other body surface. The minimum amount of drug is determined by the requirement that sufficient quantities of drug must be present in a device or composition to maintain the desired rate of release over the given period of application. The maximum amount for safety purposes is determined by the requirement that the quantity of drug present cannot exceed a rate of release that reaches toxic levels. Generally, the maximum concentration is determined by the amount of agent that can be received in the carrier without producing adverse histological effects such as irritation, an unacceptably high initial pulse of agent into the body, or adverse effects on the characteristics of the delivery device such as the loss of tackiness, viscosity, or deterioration of other properties.

Preferred classes of active agents are described below.

A. Pharmacologically Active Amines

The active agent may be a pharmacologically active nitrogen-containing base, for example, a primary amine, a secondary amine, or a tertiary amine, or it may be an aromatic or non-aromatic nitrogen-containing heterocycle, an azo compound, an imine, or a combination of any of the foregoing. Examples of specific primary amines include, but are not limited to, amphetamine, norepinephrine, phenylpropanolamine (including any of the four isomers, individually or in combination, i.e., (+)-norephedrine, (−)-norephedrine, (+)-norpseudoephedrine, and (−)-norpseudoephedrine), and pyrithiamine. Examples of secondary and tertiary amines include, but are not limited to, amiodarone, amitryptyline, azithromycin, benzphetamine, bromopheniramine, chlorambucil, chloroprocaine, chloroquine, chlorpheniramine, chlorothen, chlorpromazine, cinnarizine, clarthromycin, clomiphene, cyclobenzaprine, cyclopentolate, cyclophosphamide, dacarbazine, demeclocycline, dibucaine, dicyclomine, diethylproprion, diltiazem, dimenhydrinate, diphenhydramine, diphenylpyraline, disopyramide, doxepin, doxycycline, doxylamine, dypyridame, ephedrine, epinephrine, ethylene diamine tetraacetic acid (EDTA), erythromycin, flurazepam, gentian violet, hydroxychloroquine, imipramine, isoproterenol, isothipendyl, levomethadyl, lidocaine, loxarine, mechlorethamine, melphalan, methadone, methafurylene, methapheniline, methapyrilene, methdilazine, methotimeperazine, methotrexate, metoclopramide, minocycline, naftifine, nicardipine, nicotine, nizatidine, orphenadrine, oxybutynin, oxytetracycline, phenindamine, pheniramine, phenoxybenzamine, phentolamine, phenylephrine, phenyltoloxamine, procainamide, procaine, promazine, promethazine, proparacaine, propoxycaine, propoxyphene, pyrilamine, ranitidine, scopolamine, tamoxifen, terbinafine, tetracaine, tetracycline, thonzylamine, tranadol, triflupromazine, trimeprazine, trimethylbenzamide, trimipramine, tripelennamine, troleandomycin, uracil mustard, verapamil and vonedrine.

Examples of non-aromatic heterocyclic amines include, but are not limited to, alprazolam, amoxapine, arecoline, astemizole, atropine, azithromycin, benzapril, benztropine, beperiden, bupracaine, buprenorphine, buspirone, butorphanol, caffeine, capriomycin, ceftriaxone, chlorazepate, chlorcyclizine, chlordiazepoxide, chlorpromazine, chlorthiazide, ciprofloxacin, cladarabine, clemastine, clemizole, clindamycin, clofazamine, clonazepam, clonidine, clozapine, cocaine, codeine, cyclizine, cyproheptadine, dacarbzine, dactinomycin, desipramine, diazoxide, dihydroergotamine, diphenidol, diphenoxylate, dipyridamole, doxapram, ergotamine, estazolam, famciclovir, fentanyl, flavoxate, fludarabine, fluphenazine, flurazepam, fluvastin, folic acid, ganciclovir, granisetron, guanethidine, halazepam, haloperidol, homatropine, hydrocodone, hydromorphone, hydroxyzine, hyoscyamine, imipramine, itraconazole, keterolac, ketoconazole, levocarbustine, levorphone, lincomycin, lomefloxacin, loperamide, lorazepam, losartan, loxapine, mazindol, meclizine, meperidine, mepivacaine, mesoridazine, methdilazine, methenamine, methimazole, methotrimeperazine, methysergide, metronidazole, midazolam, minoxidil, mitomycin c, molindone, morphine, nafzodone, nalbuphine, naldixic acid, nalmefene, naloxone, naltrexone, naphazoline, nedocromil, nicotine, norfloxacin, ofloxacin, ondansetron, oxazepam, oxycodone, oxymetazoline, oxymorphone, pemoline, pentazocine, pentostatin, pentoxyfylline, perphenazine, phentolamine, physostigmine, pilocarpine, pimozide, pramoxine, prazosin, prochlorperazine, promazine, promethazine, pyrrobutamine, quazepam, quinidine, quinine, rauwolfia alkaloids, riboflavin, rifabutin, risperidone, rocuronium, scopalamine, sufentanil, tacrine, temazepam, terazosin, terconazole, terfenadine, tetrahydrazoline, thiordazine, thiothixene, ticlodipine, timolol, tolazoline, tolazamide, tolmetin, trazodone, triazolam, triethylperazine, trifluopromazine, trihexylphenidyl, trimeprazine, trimipramine, tubocurarine, vecuronium, vidarabine, vinblastine, vincristine, vinorelbine, and xylometazoline.

Examples of aromatic heterocyclic amines include, but are not limited to, acetazolamide, acyclovir, adenosine phosphate, allopurinal, alprazolam, amoxapine, amrinone, apraclonidine, azatadine, aztreonam, bisacodyl, bleomycin, brompheniramine, buspirone, butoconazole, carbinoxamine, cefamandole, cefazole, cefixime, cefinetazole, cefonicid, cefoperazone, cefotaxime, cefotetan, cefpodoxime, ceftriaxone, cephapirin, chloroquine, chlorpheniramine, cimetidine, cladarabine, clotrimazole, cloxacillin, didanosine, dipyridamole, doxazosin, doxylamine, econazole, enoxacin, estazolam, ethionamide, famciclovir, famotidine, fluconazole, fludarabine, folic acid, ganciclovir, hydroxychloroquine, iodoquinol, isoniazid, isothipendyl, itraconazole, ketoconazole, lamotrigine, lansoprazole, lorcetadine, losartan, mebendazole, mercaptopurine, methafurylene, methapyriline, methotrexate, metronidazole, miconazole, midazolam, minoxidil, nafzodone, naldixic acid, niacin, nicotine, nifedipine, nizatidine, omeperazole, oxaprozin, oxiconazole, papaverine, pentostatin, phenazopyridine, pheniramine, pilocarpine, piroxicam, prazosin, primaquine, pyrazinamide, pyrilamine, pyrimethamine, pyrithiamine, pyroxidine, quinidine, quinine, ribaverin, rifampin, sulfadiazine, sulfamethizole, sulfamethoxazole, sulfasalazine, sulfasoxazole, terazosin, thiabendazole, thiamine, thioguanine, thonzylamine, timolol, trazodone, triampterene, triazolam, trimethadione, trimethoprim, trimetrexate, triplenamine, tropicamide, and vidarabine.

Examples of azo compounds are phenazopyridine and sulfasalazine, while examples of imines include cefixime, cimetidine, clofazimine, clonidine, dantrolene, famotidine, furazolidone, nitrofurantoin, nitrofurazone, and oxiconazole.

Combinations of the aforementioned drugs and/or combinations of one or more of the aforementioned drugs with different type of active agent may also be delivered using the methods, compositions and systems of the present invention.

Examples of particularly preferred nitrogen-containing drugs include phenylpropanolamine and oxybutynin.

As many amine drugs are commercially available only in the salt form, i.e., in the form of an acid addition salt, use of a basic permeation enhancer eliminates the need to convert the drug to the free base form prior to patch manufacture. That is, the basic enhancer may be incorporated during patch manufacture, along with the acid addition salt, thus neutralizing the drug during manufacture rather than after.

B. Nonsteroidal Anti-inflammatory Agents (NSAIDs)

Suitable nonsteroidal anti-inflammatory agents that may be used in the formulations of the present invention include, but are not limited to: acetylsalicylic acid; apazone; bromfenac; celecoxib; diclofenac; difenpiramide; diflunisal; etodolac; flufenamic acid; indomethacin; ketorolac; meclofenamate; mefenamic acid; meloxicam; nabumetone; phenylbutazone; piroxicam; propionic acid derivatives (e.g., alminoprofen, benoxaprofen, butibufen, carprofen, fenbufen, fenoprofen, flurbiprofen, ibuprofen, indoprofen, ketoprofen, naproxen, oxaprozin, pirprofen, pranoprofen, suprofen, tiaprofenic acid); rofecoxib; salicylic acid; sulindac; tolmetin; and combinations of any of the foregoing. Preferred NSAIDs are ibuprofen, diclofenac (e.g., diclofenac sodium), ketoprofen, ketorolac (e.g., ketorolac tromethamine), meloxicam, piroxicam, and rofecoxib.

The NSAID or NSAIDs may be co-administered with one or more additional active agents, e.g.: antihistaminic agents such as diphenhydramine and chlorpheniramine (particularly diphenhydramine hydrochloride and chlorpheniramine maleate); corticosteroids, including lower potency corticosteroids such as alclometasone, dexamethasone, flumethasone, hydrocortisone, hydrocortisone-21-monoesters (e.g., hydrocortisone-21-acetate, hydrocortisone-21-butyrate, hydrocortisone-21-propionate, hydrocortisone-21-valerate, etc.), hydrocortisone-17,21-diesters (e.g., hydrocortisone-17,21-diacetate, hydrocortisone-17-acetate-21-butyrate, hydrocortisone-17,21-dibutyrate, etc.), prednisolone, and methylprednisolone, as well as higher potency corticosteroids such as betamethasone benzoate, betamethasone diproprionate, clobetasol propionate, diflorasone diacetate, fluocinonide, fluticasone propionate, mometasone furoate, triamcinolone acetonide, and the like; local anesthetic agents such as phenol, benzocaine, lidocaine, prilocaine and dibucaine; topical analgesics such as glycol salicylate, methyl salicylate, 1-menthol, d,l-camphor and capsaicin; and antibiotics. Preferred additional agents are antibiotic agents.

The aforementioned compounds may be administered using the methods of the invention to treat any patient with an NSAID-responsive condition or disorder. Typically, NSAIDs are employed as anti-inflammatory and/or analgesic agents, and accordingly may be used to treat individuals suffering from rheumatic or arthritic disorders, including, for example: rheumatoid arthritis, degenerative joint disease (also known as “osteoarthritis”); juvenile rheumatoid arthritis; psoriatic arthritis; gouty arthritis; ankylosing spondylitis; and lupus erythematoses such as systemic lupus erythematosus and discoid lupus erythematosus.

Other potential uses of NSAIDs, and salicylic acid in particular, include, but are not limited to, treating fever (via the anti-pyretic property of NSAIDs) or myocardial infarction, transient ischemic attacks, and acute superficial thrombophlebitis (via inhibition of platelet aggregation). Further non-limiting uses for NSAIDs include either single or adjuvant therapy for ankylosing spondylitis, bursitis, cancer-related pain, dysmenorrhea, gout, headaches, muscular pain, tendonitis, and pain associated with medical procedures such as dental, gynecological, oral, orthopedic, post-partum and urological procedures. The amount of active agent administered will depend on a number of factors and will vary from subject to subject, as noted above. Generally, however, and by way of example, a daily dosage of ketorolac using the present formulations and systems will be in the range of approximately 10-40 mg, a daily dosage of piroxicam using the present formulations and systems will be in the range of approximately 10-40 mg, and a daily dosage of ibuprofen using the present formulations and systems will be in the range of approximately 200-1600 mg/day.

The methods and compositions of the invention are expected to provide an enhanced flux of NSAIDs in the range of at least about 2- to 9-fold, preferably at least about 17- to 50-fold and most preferably at least about 86- to 128-fold, as compared to the flux observed in the absence of the basic enhancers described herein.

C. Estrogens and Progestins

Suitable estrogens that may be administered using the compositions and drug delivery systems of the invention include synthetic and natural estrogens such as: estradiol (i.e., 1,3,5-estratriene-3,17β-diol, or “17β-estradiol”) and its esters, including estradiol benzoate, valerate, cypionate, heptanoate, decanoate, acetate and diacetate; 17α-estradiol; ethinylestradiol (i.e., 17α-ethinylestradiol) and esters and ethers thereof, including ethinylestradiol 3-acetate and ethinylestradiol 3-benzoate; estriol and estriol succinate; polyestrol phosphate; estrone and its esters and derivatives, including estrone acetate, estrone sulfate, and piperazine estrone sulfate; quinestrol; mestranol; and conjugated equine estrogens. 17β-Estradiol, ethinylestradiol and mestranol are particularly preferred synthetic estrogenic agents for use in conjunction with the present invention.

Suitable progestins that can be delivered using the methods of the invention include, but are not limited to, acetoxypregnenolone, allylestrenol, anagestone acetate, chlormadinone acetate, cyproterone, cyproterone acetate, desogestrel, dihydrogesterone, dimethisterone, ethisterone (17α-ethinyltestosterone), ethynodiol diacetate, flurogestone acetate, gestadene, hydroxyprogesterone, hydroxyprogesterone acetate, hydroxyprogesterone caproate, hydroxymethylprogesterone, hydroxymethylprogesterone acetate, 3-ketodesogestrel, levonorgestrel, lynestrenol, medrogestone, medroxyprogesterone acetate, megestrol, megestrol acetate, melengestrol acetate, norethindrone, norethindrone acetate, norethisterone, norethisterone acetate, norethynodrel, norgestimate, norgestrel, norgestrienone, normethisterone, and progesterone. Progesterone, medroxyprogesterone, norethindrone, norethynodrel, d,l-norgestrel and 1-norgestrel are particularly preferred progestins.

It is generally desirable to co-administer a progestin along with an estrogen in female hormone replacement therapy so that the estrogen is not “unopposed.” As is well known, estrogen-based therapies are known to increase the risk of endometrial hyperplasia and cancer, as well as the risk of breast cancer, in treated individuals. Co-administration of estrogenic agents with a progestin has been found to decrease the aforementioned risks. Exemplary preferred combinations include, without limitation: 17β-estradiol and medroxyprogesterone acetate; 17β-estradiol and norethindrone; 17β-estradiol and norethynodrel; ethinyl estradiol and d,l-norgestrel; ethinyl estradiol and 1-norgestrel; and megestrol and medroxyprogesterone acetate.

For female HRT, it may be desirable to co-administer a small amount of an androgenic agent along with the progestin and the estrogen, in order to reproduce the complete hormone profile of the premenopausal woman, since low levels of certain androgens are present in premenopausal women. Suitable androgenic agents are discussed herein.

Any of the aforementioned steroid drugs may be naturally occurring steroids, synthetic steroids, or derivatives thereof.

Administration of a combination of steroidal active agents is useful in a variety of contexts, as will be readily appreciated by those skilled in the art. For example, the transdermal administration of a progestin with an estrogen may be used in female hormone replacement therapy, so that the symptoms or conditions resulting from altered hormone levels is mitigated or substantially prevented. The present invention is also useful to administer progestins and estrogens to treat other conditions and disorders that are responsive to topical or transdermal administration of the combination of active agents. For example, the aforementioned combination is useful to treat the symptoms of premenstrual stress and for female contraception. Exemplary combinations useful for contraception include, by way of illustration and not limitation, estradiol in combination with norethindrone acetate, and ethinyl estradiol in combination with norelgestromin.

For female hormone replacement therapy, the woman undergoing treatment will generally be of childbearing age or older, in whom ovarian estrogen, progesterone and androgen production has been interrupted either because of natural menopause, surgical procedures, radiation, chemical ovarian ablation or extirpation, or premature ovarian failure. For hormone replacement therapy, and for the other indications described herein including female contraception, the compositions or drug delivery systems are preferably used consecutively so that administration of the active agents is substantially continuous. Transdermal drug administration according to the invention provides highly effective female hormone replacement therapy. That is, the incidence and severity of hot flashes and night sweats are reduced, postmenopausal loss of calcium from bone is minimized, the risk of death from ischemic heart disease is reduced, and the vascularity and general health of the individual, is improved. Generally, the maximum concentration is determined by the amount of agent that can be received in the carrier without producing adverse histological effects such as irritation, an unacceptably high initial pulse of agent into the body, or adverse effects on the characteristics of the delivery device such as the loss of tackiness, viscosity, or deterioration of other properties. However, preferred transdermal compositions and systems for hormone replacement therapy are capable of delivering about 0.5-10.0 mg progestin, e.g., norethindrone, norethindrone acetate or the like, and about 10-200 μg estrogen, e.g., 17β-estradiol, ethinyl estradiol, mestranol or the like, over a period of about 24 hours. However, it will be appreciated by those skilled in the art that the desired dose of each individual active agent will depend on the specific active agent as well as on other factors; the minimum effective dose of each active agent is of course preferred.

The methods and compositions of the invention are expected to provide an enhanced flux of estrogens and progestins in the range of at least about 2- to 5-fold, preferably at least about 9- to 17-fold and most preferably at least about 20- to 31-fold, as compared to the flux observed in the absence of the basic enhancers described herein.

D. Androgenic Drugs

Suitable androgenic agents that may be administered using the methods, compositions and systems of the invention include, but are not limited to: the naturally occurring androgens and derivatives thereof, including androsterone, androsterone acetate, androsterone propionate, androsterone benzoate, androstenediol, androstenediol-3-acetate, androstenediol-17-acetate, androstenediol-3,17-diacetate, androstenediol-17-benzoate, androstenediol-3-acetate-17-benzoate, androstenedione, dehydroepiandrosterone (DHEA; also termed “prasterone”), sodium dehydroepiandrosterone sulfate, 4-dihydrotestosterone (DHT; also termed “stanolone”), 5α-dihydrotestosterone, dromostanolone, dromostanolone propionate, ethylestrenol, nandrolone phenpropionate, nandrolone decanoate, nandrolone furylpropionate, nandrolone cyclohexanepropionate, nandrolone benzoate, nandrolone cyclohexanecarboxylate, oxandrolone, stanozolol and testosterone; pharmaceutically acceptable derivatives (e.g., salts and esters) thereof, as well as combinations of any of the foregoing.

Pharmaceutically acceptable esters of testosterone and 4-dihydrotestosterone are of particular interest, typically esters formed from the hydroxyl group present at the C-17 position, including, but not limited to, the acetate, buciclate, caprate, cypionate, decanoate, enanthate, heptanoate, isobutyrate, isocaprate, phenylacetate, propionate, and undecanoate. Pharmaceutically acceptable derivatives of testosterone such as fluoxymesterone, methyl testosterone, oxymetholone, and testolactone, are also of interest.

Testosterone and testosterone esters, such as testosterone cypionate, testosterone enanthate, and testosterone propionate, are particularly preferred androgenic agents for use in conjunction with the present invention. The aforementioned testosterone esters are commercially available or may be readily prepared using techniques known to those skilled in the art or described in the pertinent literature.

The aforementioned androgenic agents are selected from the group consisting of naturally occurring androgens, synthetic androgens, and derivatives thereof. The active agents may be incorporated into the present dosage units and thus administered in the form of a pharmaceutically acceptable derivative, analog, ester, salt, or amide, or the agents may be modified by appending one or more appropriate functionalities to enhance selected biological properties such as penetration through the mucosal tissue. In general, with regard to androgenic agents, esters are preferred relative to salts or other derivatives. Preparation of esters, as noted herein, involves functionalization of hydroxyl and/or carboxyl groups that may be present, as will be appreciated by those skilled in the arts of pharmaceutical chemistry and drug delivery. For example, to prepare testosterone esters, the 17-hydroxyl group of the testosterone molecule is generally caused to react with a suitable organic acid under esterifying conditions, such conditions typically involving the use of a strong acid such as sulfuric acid, hydrochloric acid, or the like, and a temperature sufficient to allow the reaction to proceed at reflux. Esters can be reconverted to the free acids, if desired, by using conventional hydrogenolysis or hydrolysis procedures.

Androgenic drugs such as testosterone (17β-hydroxyandrost-4-en-3-one) are required for sperm production and promote general growth of body tissues. The primary clinical use of androgens is to replace or augment androgen secretion in hypogonadal men. Androgens may also be used to treat certain gynecologic disorders, such as to reduce breast engorgement during the postpartum period. Androgens may also be used to reduce protein loss after trauma, surgery, or prolonged immobilization, or in the treatment of anemia and hereditary angioedema. Androgens may additionally be used in the treatment of male osteoporosis or as metabolic growth stimulators in prepubertal boys.

Testosterone and its derivatives are compounds that are therapeutically effective at fairly low doses, generally in the range of approximately 5-10 mg/day.

The methods and compositions of the invention are expected to provide an enhanced flux of androgenic agent of at least about 7-fold, preferably at least about 19-fold and most preferably at least about 40-fold, as compared to the flux observed in the absence of the basic enhancers described herein.

E. Peptidyl Drugs

Peptidyl drugs that may be administered using the methods, compositions and systems of the invention include any pharmacologically active peptides, polypeptides or proteins. Once chosen, the peptidyl drug must be prepared or obtained from commercial suppliers for incorporation into a composition or delivery system. The peptidyl drug may be prepared using standard synthetic techniques, recombinant technology or extraction from natural sources.

Synthetic production of peptides, polypeptides and proteins generally employs techniques of standard solid phase peptide synthesis well known in the art. In such a method, the synthesis is sequentially carried out by incorporating the desired amino acid residues one at a time onto a growing peptide chain according to the general principles of solid phase synthesis as described, for example, by Merrifield J. Amer. Chem. Soc. 85:2149-2154(1963). Common to chemical syntheses of peptides, polypeptides and proteins is the protection of reactive side chain groups of the various amino acid moieties with suitable protecting groups, which will prevent a chemical reaction from occurring at that site until the protecting group is ultimately removed. It is also well known to protect the α-amino group on an amino acid while that entity reacts at the carboxyl group, followed by the selective removal of the α-amino protecting group to allow a subsequent reaction to take place at that site. Examples of suitable α-amino and side chain protecting groups are well known in the art.

Alternatively, the peptide, polypeptide or protein may be prepared by employing recombinant technology via techniques well known in the art. That is, conventional recombinant techniques may be used, which, as will be appreciated by those skilled in the art, involves constructing DNA encoding the desired amino acid sequence, cloning the DNA into an expression vector, transforming a host cell, e.g., a bacterial, yeast, or mammalian cell, and expressing the DNA to produce the desired peptide, polypeptide or protein.

Additionally, peptides, polypeptides or proteins can be obtained from natural sources such as a human or other animal, and may be extracted from either a living organism or from a cadaver. The material is separated and purified prior to incorporation into a drug delivery system or dosage form. Techniques of separation and purification are well known in the art and include, for example, centrifugation.

Although any peptidyl drug may be incorporated into the delivery systems of the present invention, the drug is generally selected from coagulation factors, cytokines, endorphins, kinins, hormones, LHRH (luteinizing hormone-releasing hormone) analogs and other peptidyl drugs that provide a desired pharmacological activity. Of course, the categories provided are not intended to be limiting and simply serve as a means for organization. As will be appreciated, a peptidyl drug may fall into more than one category.

Many coagulation modulators are endogenous proteins that circulate in the blood and interact with other endogenous proteins to control blood coagulation. Preferred coagulation modulators include α₁-antitrypsin, α₂-macroglobulin, antithrombin III, factor I (fibrinogen), factor II (prothrombin), factor III (tissue prothrombin), factor V (proaccelerin), factor VII (proconvertin), factor VIII (antihemophilic globulin or AHG), factor IX (Christmas factor, plasma thromboplastin component or PTC), factor X (Stuart-Power factor), factor XI (plasma thromboplastin antecedent or PTA), factor XII (Hageman factor), heparin cofactor II, kallikrein, plasmin, plasminogen, prekallikrein, protein C, protein S, thrombomodulin and combinations thereof. When applicable, both the “active” and “inactive” versions of these proteins are included.

The cytokines are a large and heterogeneous group of proteins and have a role in the function of the immune system and the control of hematopoiesis, i.e., the production of blood or blood cells. Preferred cytokines include colony stimulating factor 4, heparin binding neurotrophic factor (HBNF), interferon-α, interferon α-2a, interferon α-2b, interferon α-n3, interferon-β, interferon-γ, interleukin-1, interleukin-2, interleukin-3, interleukin-4, interleukin-5, interleukin-6, interleukin-7, interleukin-8, interleukin-9, interleukin-10, interleukin-11, interleukin-12, interleukin-13, interleukin-14, interleukin-15, interleukin-16, interleukin-17, tumor necrosis factor, tumor necrosis factor-α, granulocyte colony-stimulating factor, granulocyte-macrophage colony-stimulating factor, macrophage colony-stimulating factor, midkine, thymopoietin and combinations thereof.

Endorphins are generally peptides or small-chain peptides that activate opiate receptors. Agonist and antagonist derivatives of the naturally occurring endorphins are also contemplated. Representative examples of endorphins or pharmacologically active derivatives include dermorphin, dynorphin, α-endorphin, β-endorphin, γ-endorphin, σ-endorphin [Leu⁵]enkephalin, [Met⁵]enkephalin, substance P, and combinations thereof.

Peptidyl hormones may be naturally occurring or may be pharmacologically active derivatives of known hormones. In addition, peptidyl hormones may be human or be derived from other animal sources. Examples of peptidyl hormones that can be administered using the method, composition and delivery system of the invention include, but are not limited to, activin, amylin, angiotensin, atrial natriuretic peptide, calcitonin (derived from chicken, eel, human, pig, rat, salmon, etc.), calcitonin gene-related peptide, calcitonin N-terminal flanking peptide, cholecystokinin, ciliary neurotrophic factor, corticotropin (adrenocorticotropin hormone, corticotropin-releasing factor, epidermal growth factor, follicle-stimulating hormone, gastrin, gastrin inhibitory peptide, gastrin-releasing peptide, ghrelin, glucogon, gonadotropin-releasing factor, growth hormone releasing factor, human chorionic gonadotropin, inhibin A, inhibin B, insulin (derived from beef, human, pig, etc.), leptin, lipotropin, luteinizing hormone, luteinizing hormone-releasing hormone (LHRH), α-melanocyte-stimulating hormone, β-melanocyte-stimulating hormone, γ-melanocyte-stimulating hormone, melatonin, motilin, oxytocin (pitocin), pancreatic polypeptide, parathyroid hormone, placental lactogen, prolactin, prolactin-release inhibiting factor, prolactin-releasing factor, secretin, somatotropin, somatostatin, growth hormone-release inhibiting factor, thyrotropin (thyroid-stimulating hormone, thyrotropin-releasing factor, thyroxine, triiodothyronine, vasoactive intestinal peptide, vasopressin (antidiuretic hormone) and combinations thereof.

Particularly preferred analogues of LHRH include buserelin, deslorelin, fertirelin, goserelin, histrelin, leuprolide (leuprorelin), lutrelin, nafarelin, tryptorelin and combinations thereof.

Other examples of hormones and hormone-related drugs include anastrozle, betamethasone, bicalutamide, desmopressin, desogestrel, dexamethasone, dienestrol, drospirenone, estradiol, estropipate, ethinyl estradiol, ethynodiol diaceate, exemestane, fludrocortisone (e.g., fudrocortisone acetate), goserelin, hydrocortisone, letrozole, leuprolide (e.g., leuprolide acetate), liothyronine (e.g., liothyronine sodium), medroxyprogesterone (e.g., medroxyprogesterone acetate), methimazole, methylprednisolone (e.g., methylprednisolone acetate), methyltestosterone, norethindrone (e.g., norethindrone acetate), norgestimate, norgestrel, octreotide acetate, oxandrolone, oxymetholone, prednisolone, prednisone, progesterone, tamoxifen (e.g., tamoxifen citrate), testosterone, and toremifene (e.g., toremifene citrate).

In addition, the peptidyl drug may be a kinin. Particularly preferred kinins include bradykinin, potentiator B, bradykinin potentiator C, kallidin and combinations thereof. Still other peptidyl drugs that provide a desired pharmacological activity can be incorporated into the delivery systems of the invention. Examples include abarelix, adenosine deaminase, anakinra, ancestim, alteplase, alglucerase, asparaginase, bivalirudin, bleomycin, bombesin, desmopressin acetate, des-Q14-ghrelin, domase-α, enterostatin, erythropoeitin, exendin-4, fibroblast growth factor-2, filgrastim, β-glucocerebrosidase, gonadorelin, hyaluronidase, insulinotropin, lepirudin, magainin I, magainin II, nerve growth factor, pentigetide, thrombopoietin, thymosin α-1, thymidin kinase, tissue plasminogen activator, tryptophan hydroxylase, urokinase, urotensin II and combinations thereof.

Particularly preferred systemically active agents that can be administered transdermally in conjunction with the present invention include oxytocin, insulin and LHRH analogues, such as leuprolide.

The methods and compositions of the invention are expected to provide an enhanced flux of peptidyl drugs in the range of at least about 6- to 9-fold, preferably at least about 27- to 34-fold, as compared to the flux observed in the absence of the basic enhancers described herein.

F. Locally Administered Active Agents

Preferred agents for local, topical administration are within the broad classes of compounds known to be topically administrable, including, but not limited to, topical antibiotics (e.g., magainin I and magainin II), anti-acne agent, anti-fungal agents, anti-psoriatic agents, antipruritic agents, antihistamines, antineoplastic agents (e.g., asparaginase and bleomycin), local anesthetics, anti-inflammatory agents and the like.

Suitable topical antibiotic agents include, but are not limited to, antibiotics of the lincomycin family (referring to a class of antibiotic agents originally recovered from streptomyces lincolnensis); antibiotics of the tetracycline family (referring to a class of antibiotic agents originally recovered from streptomyces aureofaciens); sulfur-based antibiotics, i.e., sulfonamides; mupirocin; and antibiotics such as magainin I and magainin II. Exemplary antibiotics of the lincomycin family include lincomycin itself (6,8-dideoxy-6-[[(1-methyl-4-propyl-2-pyrrolidinyl)-carbonyl]amino]-1-thio-L-threo-α-D-galacto-octopyranoside), clindamycin, the 7-deoxy, 7-chloro derivative of lincomycin (i.e., 7-chloro-6,7,8-trideoxy-6-[[(1-methyl-4-propyl-2-pyrrolidinyl)carbonyl]-amino]-1-thio-L-threo-α-D-galacto-octopyranoside), related compounds as described, for example, in U.S. Pat. Nos. 3,475,407, 3,509,127, 3,544,551 and 3,513,155, and pharmacologically acceptable salts and esters thereof. Exemplary antibiotics of the tetracycline family include tetracycline itself (4-(dimethylamino)-1,4,4α,5,5α,6,11,12α-octahydro-3,6,12,12α-pentahydroxy-6-methyl-1,11-dioxo-2-naphthacene-carboxamide), chlortetracycline, oxytetracycline, tetracycline, demeclocycline, rolitetracycline, methacycline and doxycycline and their pharmaceutically acceptable salts and esters, particularly acid addition salts such as the hydrochloride salt. Exemplary sulfur-based antibiotics include, but are not limited to, the sulfonamides sulfacetamide, sulfabenzamide, sulfadiazine, sulfadoxine, sulfamerazine, sulfamethazine, sulfamethizole, sulfamethoxazole, and pharmacologically acceptable salts and esters thereof, e.g., sulfacetamide sodium.

Topical anti-acne agents include adapalene, azelaic acid, benzoyl peroxide, clindamycin and clindamycin phosphate, doxycycline, erythromycin, keratolytics such as salicylic acid and retinoic acid (Retin-A”), norgestimate, organic peroxides, retinoids such as isotretinoin and tretinoin, sulfacetamide sodium, and tazarotene. Preferred anti-acne agents include adapalene, azelaic acid, benzoyl peroxide, clindamycin (e.g., clindamycin phosphate), doxycycline (e.g., doxycycline monohydrate), erythromycin, isotretinoin, norgestimate, sulfacetamide sodium, tazarotene, and tretinoin.

Exemplary topical antifungal agents include amphotericin B, benzoic acid, butenafine and butenafine HCl, butoconazole and butoconazole nitrate, caprylic acid, chloroxylenol, ciclopirox, clotrimazole, econazole and econazole nitrate, fluconazole, itraconazole, ketoconazole, miconazole and miconazole nitrate, naftifine and naftifine HCl, nystatin, oxiconazole and oxiconazole nitrate, salicylic acid, selenium and selenium sulfide, sulconazole and sulconazole nitrate, terbinafine and terbinafine HCl, terconazole, tioconazole, and undecylenic acid.

Topical antipsoriatic agents include acitretin, alclometasone dipropionate, anthralin, azathioprine, calcipotriene, calcitriol, colchicine, cyclosporine, methoxsalen, retinoids, and vitamin A.

Exemplary local anesthetics include alcohols such as phenol; benzyl benzoate; calamine; chloroxylenol; dyclonine; ketamine; menthol; pramoxine; resorcinol; troclosan; and procaine drugs such as benzocaine, bupivacaine, chloroprocaine, cinchocaine, cocaine, dexivacaine, diamocaine, dibucaine, etidocaine, hexylcaine, levobupivacaine, lidocaine, mepivacaine, oxethazaine, prilocaine, procaine, proparacaine, propoxycaine, pyrrocaine, risocaine, rodocaine, ropivacaine, and tetracaine; and combinations thereof. Derivatives of these compounds, such as pharmaceutically acceptable salts and esters are also of particular interest, for example, bupivacaine HCl, chloroprocaine HCl, diamocaine cyclamate, dibucaine HCl, dyclonine HCl, etidocaine HCl, levobupivacaine HCl, lidocaine HCl, mepivacaine HCl, pramoxine HCl, prilocaine HCl, procaine HCl, proparacaine HCl, propoxycaine HCl, ropivacaine HCl, and tetracaine HCl, and so forth. Preferred local anesthetics include bupivacaine, chloroprocaine, dibucaine, etidocaine, levobupivacaine, lidocaine, mepivacaine, prilocaine, ropivacaine, tetracaine, and pharmaceutically acceptable salts and esters thereof.

The methods and compositions of the invention are expected to provide an enhanced flux of local anesthetics of at least about 1.5-fold, preferably at least about 3-fold, as compared to the flux observed in the absence of the basic enhancers described herein.

Exemplary anti-inflammatory agents include topical corticosteroids, and may be one of the lower potency corticosteroids such as hydrocortisone, hydrocortisone-21-monoesters (e.g., hydrocortisone-21-acetate, hydrocortisone-21-butyrate, hydrocortisone-21-propionate, hydrocortisone-21-valerate, etc.), hydrocortisone-17,21-diesters (e.g., hydrocortisone-17,21-diacetate, hydrocortisone-17-acetate-21-butyrate, hydrocortisone-17,21-dibutyrate, etc.), alclometasone, dexamethasone, flumethasone, prednisolone, or methylprednisolone, or may be a higher potency corticosteroid such as clobetasol propionate, betamethasone benzoate, betamethasone diproprionate, diflorasone diacetate, fluocinonide, mometasone furoate, triamcinolone acetonide, or the like.

G. ADD and ADHD Drugs

Suitable attention deficit disorder (ADD) and attention deficit hyperactivity disorder (ADHD) drugs that may be administered using the methods, compositions and systems of the invention include, but are not limited to: antihypertensive agents such as clonidine and guanfacine; and stimulants such as dextroamphetamine, methylphenidate, and pemoline. Derivatives of these compounds, such as pharmaceutically acceptable salts and esters are also of particular interest, for example, dextroamphetamine sulfate and methylphenidate HCl.

H. Alzheimer's Drugs.

Suitable drugs for the treatment of Alzheimer's disease that may be administered using the methods, compositions and systems of the invention include, but are not limited to: donepezil, galanthamine, rivastigmine, and tacrine. Derivatives of these compounds, such as pharmaceutically acceptable salts and esters are also of particular interest, for example, donepezil HCl, galanthamine HBr, rivastignine tartrate, and tacrine HCl.

The methods and compositions of the invention are expected to provide an enhanced flux of Alzheimer's drugs of at least about 2-fold, preferably at least about 3-fold, as compared to the flux observed in the absence of the basic enhancers described herein.

I. Anti-Anxiety Drugs.

Suitable anti-anxiety drugs that may be administered using the methods, compositions and systems of the invention include, but are not limited to: adatanserin hydrochloride, alpidem, binospirone mesylate, bretazenil, buspirone, benzodiazepines (e.g., alprazolam, chlordiazepoxide, clonazepam, clorazepate, diazepam, estazolam, flurazepam, lorazepam, olanzapine, oxazepam, quazepam, temazepam, and triazolam), glemanserin, ipsapirone hydrochloride, mirisetron maleate, ocinaplon, ondansetron hydrochloride, panadiplon, pancopride, pazinaclone, serazapine hydrochloride, tandospirone citrate, zalospirone hydrochloride. Derivatives of these compounds, such as pharmaceutically acceptable salts and esters are also of particular interest, examples of which are listed above. Preferred anti-anxiety drugs include benzodiazepines, and alprazolam, clonazepam, lorazepam and olanzapine, in particular.

J. Anti-Arthritic Drugs

Suitable anti-arthritic drugs that may be administered using the methods, compositions and systems of the invention include, but are not limited to: glucosamine, chondroitin sulfate, COX-2 inhibitors, and combinations thereof. Derivatives of these compounds, such as pharmaceutically acceptable salts and esters are also of particular interest, for example glucosamine sulfate.

K. Anti-Asthmatic Agents and Other Respiratory Drugs

Suitable anti-asthmatic agents that may be administered using drugs that may be administered using the methods, compositions and systems of the invention include, but are not limited to: ablukast, azelastine, bunaprolast, cinalukast, cromolyn, crornitrile, enofelast, isamoxole, ketotifen fumarate, levcromakalim, lodoxamide ethyl, lodoxamide tromethamine, montelukast, ontazolast, oxarbazole, oxatomide, piriprost, pirolate, pobilukast edamine, quazolast, repirinast, ritolukast, salmeterol xinafoate, sulukast, tetrazolast meglumine, tiaramide, tibenelast, tomelukas, tranilast, verlukast, verofylline, and zarirlukast. Other respiratory drugs that can be administered, include, but are not limited to: albuterol, aminophylline, formoterol, nikethamide, oxytriphylline, terbutaline, theophylline, and other xanthine derivatives. Preferred anti-asthmatic agents include albuterol, cromolyn and terbutaline.

Derivatives of these compounds, such as pharmaceutically acceptable salts and esters are also of particular interest, for example, ablukast sodium, albuterol sulfate, azelastine hydrochloride, cromolyn sodium, crornitrile sodium, montelukast sodium, piriprost potassium, terbutaline sulfate, tiaramide hydrochloride, and tibenelast sodium.

L. Anticholinergic/Antispasmodic Drugs

Suitable anticholinergic/antispasmodic drugs that may be administered using the methods, compositions and systems of the invention include, but are not limited to: anisotropine, atropine, belladonna, clidinium, dicyclomine, glycopyrolate, homatropine, hyoscyamine, mepenzolate, methantheline, methscopolamine, oxybutynin, pirenzepine, propantheline, scopolamine, tolteridine.

Derivatives of these compounds, such as pharmaceutically acceptable salts and esters are also of particular interest, for example oxybutynin chloride and tolterodine tartrate.

Of particular interest is oxybutynin, which is commonly used in treating individuals suffering from an overactive bladder, e.g., neurogenic bladder (Guittard et al., U.S. Pat. No. 5,674,895). Oxybutynin contains a chiral center, and may therefore be administered as either a racemate or a single isomer. There is some disagreement as to whether the activity of the racemate resides in the S enantiomer or the R enantiomer, it appears that the activity predominantly resides in the R enantiomer (Noronha-Blob, J. Pharmacol. Exp. Ther. 256(2):562-567 (1990) and Goldenberg, Clin Ther. 21(4):634-642 (1999)). U.K. Patent No. 940,540 describes the preparation of racemic oxybutynin. Synthesis of (S)-oxybutynin is also known. For example, the S enantiomer may be obtained by resolution of the intermediate mandelic acid followed by esterification (Kachur et al., J. Pharmacol. Exp. Ther. 247(3):867-72(1988)). The R enantiomer may obtained by first preparing 4-diethylamino-2-butynyl chloride from dichlorobutyne followed by reacting the single R enantiomer of cyclohexylphenylglycolic acid with the prepared 4-diethylamino-2-butynyl chloride to yield the R enantiomer of 4-diethylamino-2-butynyl phenylcyclohexlglycolate, i.e., (R)-oxybutynin (Aberg, U.S. Pat. No. 6,123,961). Alternatively, the individual isomers may be isolated from a racemic mixture of oxybutynin using techniques known in the art such as chromatography-based methods that use a chiral substrate. Transdermal administration of oxybutynin is useful in a variety of contexts, as will be readily appreciated by those skilled in the art. For example, the transdermal administration of oxybutynin is useful in the treatment of urinary urgency, urinary frequency, urinary leakage, incontinence, and painful or difficult urination. Generally, although not necessarily, these disorders are caused by a neurogenic bladder. In addition, the present compositions and drug delivery systems are useful to administer oxybutynin to treat other conditions and disorders that are responsive to transdermal administration of oxybutynin. For example, oxybutynin may be administered transdermally to treat individuals suffering from detrusor hyperreflexia and detrusor instability. Generally, a daily dosage of racemic oxybutynin using the present formulations and delivery systems will be in the range of about 1-20 mg over a 24-hour period. The daily dose of an individual enantiomer of oxybutynin, i.e., (S)-oxybutynin or (R)-oxybutynin, using the present formulations and delivery systems is preferably lower than the corresponding racemate dose. Specifically, it is preferred that the enantiomer dose be in the range of about 0.5-15 mg over a 24-hour period.

The methods and compositions of the invention are expected to provide an enhanced flux of anticholinergic drugs/antispasmodic drugs of at least about 1.5-fold.

M. Antidepressant Drugs

Suitable antidepressant drugs that may be administered using the methods, compositions and systems of the invention include, but are not limited to: adatanserin hydrochloride, adinazolam and adinazolam mesylate, alaproclate, aletamine hydrochloride, amedalin hydrochloride, amitriptyline and amitriptyline hydrochloride, amoxapine, aptazapine maleate, azaloxan fumarate, azepindole, azipramine hydrochloride, bipenamol hydrochloride, bupropion and bupropion hydrochloride, buspirone, butacetin, butriptyline hydrochloride, caroxazone, cartazolate, chlordiazepoxide, ciclazindol, cidoxepin hydrochloride, cilobamine mesylate, citalopram, clodazon hydrochloride, clomipramine and clomipramine hydrochloride, cotinine fumarate, cyclindole, cypenamine hydrochloride, cyprolidol hydrochloride, cyproximide, daledalin tosylate, dapoxetine hydrochloride, dazadrol maleate, dazepinil hydrochloride, desipramine and desipramine hydrochloride, dexamisole, deximafen, dibenzepin hydrochloride, dioxadrol hydrochloride, dothiepin hydrochloride, doxepin and doxepin hydrochloride, duloxetine hydrochloride, eclanamine maleate, encyprate, etoperidone hydrochloride, fantridone hydrochloride, fehmetozole hydrochloride, fenmetramide, fezolamine fumarate, fluotracen hydrochloride, fluoxetine and fluoxetine hydrochloride, fluparoxan hydrochloride, fluvoxamine, gamfexine, guanoxyfen sulfate, imafen hydrochloride, imiloxan hydrochloride, imipramine and imipramine hydrochloride, indeloxazine hydrochloride, intriptyline hydrochloride, iprindole, isocarboxazid, ketipramine fumarate, lofepramine hydrochloride, lortalamine, maprotiline and maprotiline hydrochloride, melitracen hydrochloride, milacemide hydrochloride, minaprine hydrochloride, mirtazapine, moclobemide, modaline sulfate, napactadine hydrochloride, napamezole hydrochloride, nefazodone and nefazodone hydrochloride, nisoxetine, nitrafudam hydrochloride, nomifensine maleate, nortriptyline and nortriptyline hydrochloride, octriptyline phosphate, opipramol hydrochloride, oxaprotiline hydrochloride, oxypertine, paroxetine, perphenazine, phenelzine and phenelzine sulfate, pirandamine hydrochloride, pizotyline, pridefine hydrochloride, prolintane hydrochloride, protriptyline and protriptyline hydrochloride, quipazine maleate, rolicyprine, seproxetine hydrochloride, sertraline and sertraline hydrochloride, sibutramine hydrochloride, sulpiride, suritozole, tametraline hydrochloride, tampramine fumarate, tandamine hydrochloride, thiazesim hydrochloride, thozalinone, tomoxetine hydrochloride, tranylcypromine, trazodone and trazodone hydrochloride, trebenzomine hydrochloride, trimipramine and trimipramine maleate, venlafaxine and venlafaxine hydrochloride, viloxazine hydrochloride, zimeldine hydrochloride, zometapine, and pharmaceutically acceptable derivatives thereof, and combinations thereof.

Derivatives of these compounds, such as pharmaceutically acceptable salts and esters are also of particular interest, for example, amitriptyline HCl, citalopram HBr, doxepin HCl, fluoxetine HCl, fluvoxamine maleate, paroxetine HCl, phenelzine sulfate, protriptyline HCl, sertraline HCl, tranylcypromine sulfate, venlafaxine HCl, as well as those included in the list above.

Preferred antidepressants include monoamine oxidase inhibitors such as phenelzine and tranylcypromine; selective serotonin reuptake inhibitors such as citalopram, fluoxetine, fluvoxamine, nefazodone, paroxetine, sertraline, and venlafaxine; tricyclic anti-depressants such as amitriptyline, amoxapine, clomipramine, desipramine, doxepin, imipramine, maprotiline, mirtazapine, nortriptyline, protriptyline, and trimipramine; other anti-depressants such as bupropion, buspirone, chlordiazepoxide, perphenazine, and trazodone.

More preferred antidepressant drugs include the monoamine oxidase inhibitors: phenelzine and tranylcypromine; the selective serotonin reuptake inhibitors: citalopram, fluoxetine, paroxetine, sertraline, and venlafaxine; the tricyclic anti-depressant: amitriptyline, doxepin, mirtazapine, and protriptyline; and other anti-depressants such as chlordiazepoxide, and perphenazine.

The methods and compositions of the invention are expected to provide an enhanced flux of antidepressant drugs of at least about 2-fold, preferably at least about 6-fold, as compared to the flux observed in the absence of the basic enhancers described herein.

N. Antihypertensive Agents

Suitable antihypertensive agents that may be administered using the methods, compositions and systems of the invention include, but are not limited to: alfuzosin hydrochloride, alipamide, althiazide, amiloride, amiquinsin hydrochloride, amlodipine and amlodipine besylate, amlodipine maleate, anaritide acetate, atenolol, atiprosin maleate, belfosdil, bemitradine, benazepril, benazeprilat, bendacalol mesylate, bendroflumethiazide, benzthiazide, betaxolol hydrochloride, bethanidine sulfate, bevantolol hydrochloride, biclodil hydrochloride, bisoprolol, bisoprolol fumarate, bucindolol hydrochloride, bupicomide, buthiazide, candoxatril, candoxatrilat, captopril, carvedilol, ceronapril, chlorothiazide sodium, cicletanine, cilazapril, clonidine and clonidine hydrochloride, clopamide, cyclopenthiazide, cyclothiazide, darodipine, debrisoquin sulfate, delapril hydrochloride, diapamide, diazoxide, dilevalol hydrochloride, diltiazem and diltiazem malate, ditekiren, doxazosin and doxazosin mesylate, ecadotril, enalapril and enalapril maleate, enalaprilat, enalkiren, endralazine mesylate, epithiazide, eprosartan, eprosartan mesylate, felodipine, fenoldopam mesylate, flavodilol maleate, flordipine, flosequinan, fosinopril and fosinopril sodium, fosinoprilat, furosemide, guanabenz and guanabenz acetate, guanacline sulfate, guanadrel sulfate, guancydine, guanethidine monosulfate and guanethidine sulfate, guanfacine and guanfacine hydrochloride, guanisoquin sulfate, guanoclor sulfate, guanoctine hydrochloride, guanoxabenz, guanoxan sulfate, guanoxyfen sulfate, hydralazine hydrochloride, hydralazine polistirex, hydrochlorothiazide, hydroflumethiazide, indacrinone, indapamide, indolaprif hydrochloride, indoramin, indoramin hydrochloride, indorenate hydrochloride, isradipine, lacidipine, leniquinsin, levcromakalim, lisinopril, lofexidine hydrochloride, losarten and losartan potassium, losulazine hydrochloride, mebutamate, mecamylamine hydrochloride, medroxalol, medroxalol hydrochloride, methalthiazide, methyclothiazide, methyldopa, methyldopate hydrochloride, metipranolol, metolazone, metoprolol, metoprolol fumarate, metoprolol succinate, metyrosine, minoxidil, monatepil maleate, muzolimine, nadolol, nebivolol, nicardipine, nifedipine, nimodipine, nitrendipine, oformine, pargyline hydrochloride, pazoxide, pelanserin hydrochloride, perindopril and perindopril erbumine, perindoprilat, phenoxybenzamine and phenoxybenzamine hydrochloride, pinacidil, pivopril, polythiazide, prazosin and prazosin hydrochloride, primidolol, prizidilol hydrochloride, propranolol, quinapril and quinapril hydrochloride, quinaprilat, quinazosin hydrochloride, quinelorane hydrochloride, quinpirole hydrochloride, quinuclium bromide, ramipril, ramiprilat, rauwolfia serpentina, reserpine, saprisartan potassium, saralasin acetate, sodium nitroprusside, spironolactone, sulfinalol hydrochloride, tasosartan, teludipine hydrochloride, temocapril hydrochloride, terazosin and terazosin hydrochloride, terlakiren, tiamenidine, tiamenidine hydrochloride, ticrynafen, tinabinol, tiodazosin, timolol, tipentosin hydrochloride, trichlormethiazide, trimazosin hydrochloride, trimethaphan camsylate, trimoxamine hydrochloride, tripamide, verapamil, xipamide, zankiren hydrochloride, zofenoprilat arginine; pharmaceutically acceptable derivatives thereof, and combinations thereof.

Of particular interest are α-adrenergic antagonists such as doxazosin, phenoxybenzamine, prazosin, and terazosin; angiotensin converting enzyme inhibitors such as benazepril, benazeprilat, enalapril, enalaprilat, fosinopril, fosinoprilat, lisinopril, perindopril, perindoprilat, quinapril, quinaprilat, ramipril, and ramiprilat; β-blockers such as carvedilol, nadolol, and timolol; and calcium channel blockers such as amlodipine, felodipine, isradipine, nicardipine, nifedipine, and nimodipine; as well as pharmaceutically acceptable derivatives thereof.

Derivatives of the aforementioned compounds, such as pharmaceutically acceptable salts and esters are also of particular interest, for example, doxazosin mesylate, enalapril maleate, fosinopril sodium, losartan potassium, prazosin HCl, terazosin HCl, as well as those included in the list above.

The methods and compositions of the invention are expected to provide an enhanced flux of antihypertensive agents of at least about 30-fold, preferably at least about 50-fold, and more preferably about 63-fold, as compared to the flux observed in the absence of the basic enhancers described herein.

O. Antiparkinsonism Drugs

Suitable antiparkinsonism drugs that may be administered using the methods, compositions and systems of the invention include, but are not limited to: anticholinergics such as amantadine, benztropine, beperiden, cycrimine, procyclidine, and trihexyphenidyl; antidyskinetics such as selegiline; cabergoline; COMT inhibitors such as entacapone and tolcapone, both of which are administered with levodopa/carbidopa; diphenhydramine; dopamine receptor agonists such as bromocriptine, levodopa/carbidopa, metoclopramide, pergolide, pramipexole and ropinirole; as well as other compounds such as diphenhydramine and hyoscyamine.

Preferred antiparkinsonism drugs include benztropine, biperiden, bromocriptine, carbidopa, levodopa, diphenhydramine, hyoscyamine, pergolide, pramipexole, ropinirole, selegiline, and trihexyphenidyl.

Derivatives of these compounds, such as pharmaceutically acceptable salts and esters are also of particular interest, for example, amantadine HCl, benztropine mesylate, biperiden HCl, bromocriptine mesylate, carbidopa, levodopa, diphenhydramine HCl, hyoscyamine sulfate, pergolide mesylate, pramipexole dihydrochloride, ropinirole HCl, selegiline HCl, and trihexyphenidyl HCl.

P. Antipsychotic Agents

Suitable antipsychotics agents that may be administered using the methods, compositions and systems of the invention include, but are not limited to: acetophenazine maleate, alentemol hydrobromide, alpertine, azaperone, batelapine maleate, benperidol, benzindopyrine hydrochloride, brofbxine, bromperidol and bromperidol decanoate, buspirone, butaclamol hydrochloride, butaperazine and butaperazine maleate, carphenazine maleate, carvotroline hydrochloride, chlorpromazine and chlorpromazine hydrochloride, chlorprothixene, cinperene, cintriamide, clomacran phosphate, clopenthixol, clopimozide, clopipazan mesylate, cloroperone hydrochloride, clothiapine, clothixamide maleate, clozapine, cyclophenazine hydrochloride, droperidol, etazolate hydrochloride, fenimide, flucindole, flumezapine, fluphenazine decanoate and fluphenazine enanthate and fluphenazine hydrochloride, fluspiperone, fluspirilene, flutroline, gevotroline hydrochloride, halopemide, haloperidol and haloperidol decanoate, iloperidone, imidoline hydrochloride, lenperone, loxapine, mazapertine succinate, mesoridazine and mesoridazine besylate, metiapine, milenperone, milipertine, molindone and molindone hydrochloride, naranol hydrochloride, neflumozide hydrochloride, ocaperidone, olanzapine, oxiperomide, penfluridol, pentiapine maleate, perphenazine, pimozide, pinoxepin hydrochloride, pipamperone, piperacetazine, pipotiazine palniitate, piquindone hydrochloride, prochlorperazine and prochlorperazine edisylate and prochlorperazine maleate, promazine hydrochloride, quetiapine and quetiapine fumarate, remoxipride and remoxipride hydrochloride, rimcazole hydrochloride, risperidone, seperidol hydrochloride, sertindole, setoperone, spiperone, thioridazine and thioridazine hydrochloride, thiothixene and thiothixene hydrochloride, tioperidone hydrochloride, tiospirone hydrochloride, trifluroperazine and trifluoperazine hydrochloride, trifluperidol, triflupromazine and triflupromazine hydrochloride, ziprasidone and ziprasidone hydrochloride.

Derivatives of these compounds, such as pharmaceutically acceptable salts and esters are also of particular interest, for example, trifluoperazine HCl, as well as those included in the list above.

Preferred antipsychotics are a typical antipsychotic agents (i.e., drugs that act on different neurotransmitters than the conventions antipsychotic agents) such as clozapine, olanzapine, quetiapine fumarate, and risperidone; conventional antipsychotic agents such as chlorpromazine, fluphenazine, haloperidol, loxapine, mesoridazine, perphenazine, pimozide, prochlorperazine (and other phenothiazines), thiothixene and trifluroperazine; and related drugs such as buspirone.

Most preferred antipsychotic agents include buspirone, olanzapine, pimozide, prochlorperazine, risperidone and trifluroperazine.

The methods and compositions of the invention are expected to provide an enhanced flux of antipsychotic agents of at least about 15-fold, preferably at least about 35-fold, as compared to the flux observed in the absence of the basic enhancers described herein.

Q. Bone Density Regulators

Suitable bone density regulators that may be administered using the methods, compositions and systems of the invention include, but are not limited to: alendronate, calcitonin, etidronate, pamidronate, raloxifene, risedronate, and tiludronate. Derivatives of these compounds, such as pharmaceutically acceptable salts and esters are also of particular interest, for example, alendronate sodium, etidronate sodium and etidronate disodium, pamidronate disodium, raloxifene HCl, risedronate sodium, and tiludronate sodium. Preferred bone density regulators include alendronate, etidronate, raloxifene, and risedronate, tiludronate, and pharmaceutically acceptable derivatives thereof.

The methods and compositions of the invention are expected to provide an enhanced flux of bone density regulators of at least about 2-fold, preferably at least about 3-fold, as compared to the flux observed in the absence of the basic enhancers described herein.

R. Analgesic Agents

Suitable analgesic agents that may be administered using the methods, compositions and systems of the invention include, but are not limited to: capsaicin, indomethacin, and centrally acting analgesics such as clonidine and tramadol. Of particular interest are narcotic analgesics or narcotic “painkillers”, examples of which include, by way of illustration, alfentanil, buprenorphine, butorphanol, codeine, enkephalin, fentanyl, hydrocodone, hydromorphone, levorphanol, meperidine, methadone, morphine, nicomorphine, opium, oxycodone, oxymorphone, pentazocine, propoxyphene, and sufentanil.

Preferred analgesic agents include buprenorphine, butorphanol, fentanyl, hydrocodone, hydromorphone, levorphanol, methadone, morphine, oxycodone, and oxymorphone.

Derivatives of these compounds, such as pharmaceutically acceptable salts and esters are also of particular interest, for example, buprenorphine HCl, clonidine HCl, hydrocodone bitartrate, hydromorphone HCl, levorphanol tartrate, methadone HCl, morphine sulfate, and oxymorphone HCl.

The methods and compositions of the invention are expected to provide an enhanced flux of analgesic agents of at least about 3-fold, preferably at least about 6-fold, as compared to the flux observed in the absence of the basic enhancers described herein.

S. Sympathomimetic Drugs

Suitable sympathomimetic drugs that may be administered using the methods, compositions and systems of the invention include, but are not limited to: phenylpropanolamine. Derivatives of these compounds, such as pharmaceutically acceptable salts and esters are also of particular interest, for example, phenylpropanolamine HCl.

Phenylpropanolamine, or 2-amino-1-phenyl-1-propanol, is of particular interest and is described, for example, by Kanfer et al., in Analytical Profiles of Drug Substances, vol. 12, K. Florey, Ed. (New York: Academic Press, 1983). Phenylpropanolamine has been used as an anorectic agent, a decongestant, an anxiolytic agent, and as a drug for decreasing fatigue and confusion. See, for example, U.S. Pat. Nos. 5,019,594 to Wurtman et al., 5,260,073 to Phipps, and 5,096,712 to Wurtman. Phenylpropanolamine has two chiral centers and thus exists as four different isomers, generally referred to as (+)-norephedrine, (−)-norephedrine, (+)-norpseudoephedrine, and (−)-norpseudoephedrine, respectively. Generally, (−)-norephedrine and (+)-norpseudoephedrine are recognized as the more active isomers for most physiological uses. Phenylpropanolamine may be transdermally herein as a racemate, i.e., as a mixture of any two or more of the four isomers of phenylpropanolamine, generally a racemic mixture of (−)-norephedrine and (+)-norephedrine, or any one of the four isomers may be administered individually. Phenylpropanolamine will usually be administered as an anorectic agent (i.e., for appetite suppression), or may be employed as a decongestant, as an anxiolytic agent, or to decrease fatigue and confusion. Most commonly, the drug is used as either an anorectic agent or a decongestant. Generally, a daily dosage of racemic phenylpropanolamine using the present formulations and delivery systems will be in the range of about 10 mg/day to about 250 mg/day, preferably about 25 mg/day to about 200 mg/day.

The methods and compositions of the invention are expected to provide an enhanced flux of sympathomimetic drugs in the range of at least about 2- to 6-fold, preferably at least about 14-fold and most preferably at least about 20-fold, as compared to the flux observed in the absence of the basic enhancers described herein.

T. Cholesterol-Lowering Agents

Suitable cholesterol-lowering agents that may be administered using the methods, compositions and systems of the invention include, but are not limited to: simvastatin. Derivatives of these compounds, such as pharmaceutically acceptable salts and esters are also of particular interest.

U. Other Active Agents and Analogs

Still other examples of systemically active agents for which the transdermal formulations and drug delivery systems of the invention are preferred include, but are not limited to, the following:

antibiotics including antibacterial agents: clindamycin phosphate;

anticancer agents: paclitaxel, tamoxifen, and antineoplastic drugs such as anti-metabolites (e.g., cladribine and thioguanine), anti-estrogens (e.g., anastrozole and letrozole) and fluorouracil;

anticholinergic antagonists: hyoscyamine sulfate;

anticonvulsants: clorazepate dipotassium, lamotrigine, and lorazepam;

antidiabetic agents: repaglinide, and rosiglitazone maleate;

anti-emetics: chlorpromazine HCl, dronabinol, granisetron HCl, meclizine HCl, metoclopramide, ondansetron HCl, perphenazine, prochlorperazine, promethazine, and scopolamine;

antihistamines: azelastine hydrochloride, cetirizine and cetirizine HCl, cyproheptadine HCl, dexbrompheniramine maleate, fexofenadine and fexofenadine HCl, loratadine, triprolidine, tripelenamine and diphenhydramine;

antilipemic agents: HMG-CoA reductase inhibitors such as atorvastatine, fluvastatin, lovastatine, pravastatin and simvastatine;

antimigraine preparations: dihydroergotamine mesylate, naratriptan, sumatriptan succinate, timolol, and zolmitriptan;

antinauseants: granisetron and ondansetron;

antipanic agents: alprazolam, clonazepam, paroxetine hydrochloride, sertraline, and sertraline HCl;

antirheumatic agents: leflunomide, methotrexate and NSAIDs;

anti-ulcer agents: ameprazole, famotidine, lansoprazole and omerprazole;

antiviral agents: acyclovir, penciclovir, rimantadine hydrochloride, zanamivir, as well as nucleoside analogues such as stavudine and zalcitabine;

appetite stimulant: dronabinol;

appetite suppressants: sibutramine, phenylpropanolamine and obesity management drugs such as methamphetamine (e.g., methamphetamine hydrochloride), phendimetrazine tartrate, phentermine (e.g., phentermine hydrochloride), and sibutramine (e.g., sibutramine hydrochloride monohydrate);

antitussive agents: dextromethorphan hydrobromide;

benign prostatic hyperplasia agents: doxazosin (e.g., doxazosin mesylate), finasteride, tamsulosin, terazosin (e.g., terazosin HCl);

cardiovascular preparations: angiotensin converting enzyme inhibitors such as 3-(5-amino-1-carboxy-1S-pentyl)amino-2,3,4,5-tetrahydro-2-oxo-3 S-1H-1-benzazepine-1-acetic acid, 1-carboxymethyl-3-1-carboxy-3-phenyl-(1S)-propylamino-2,3,4,5-tetrahydro-1H-(3S)-1-benzazepine-2-one, enalapril, 3-(1-ethoxycarbonyl-3-phenyl-(IS)-propylamino)-2,3,4,5-tetrahydro-2-oxo-(3S)-benzazepine-1-acetic acid monohydrochloride and lisinorpril; angiotensin III receptor antagonists such as candesartan, cilexetil, losartan potassium, valsartan, and telmisartan; anti-arrhythmics such as amiodarone, bretylium, disopyramide, digoxin, dofetilide, encainide, flecainide, ibutilide and ibutilide fumarate, lidocaine, mexiletine, moricizine, phenytoin, procainamide, quinidine, and tocainide; antiplatelet drugs such as anagrelide HCl, clopidogrel bisulfate, epoprostenol sodium, tirofiban HCl; beta-blockers such as acebutolol, atenolol, esmolol, metoprolol, pindolol, propafenone, propranolol, and sotalol; cardiac glycosides such as digoxin and digitoxin; cardioprotective agents such as dexrazoxane and leucovorin; vasodilators such as nitroglycerin; cholinergic agents such as arecoline; diuretics; pre- and afterload reducers; inotropes such as amrinone and milrinone; calcium channel blockers such as verapamil, nifedipine, nicardipene, felodipine, isradipine, nimodipine, bepridil, amlodipine and diltiazem;

central nervous system agents: bromocriptine, ±trans-1,3,4,4α,5,10β-hexahydro-4-propyl-2H-1-benzopyrano-3,4-bipyridine-9-ol monohydrochloride, zolpidem tartrate;

central nervous system stimulants: amphetamine, dextroamphetamine, doxapram HCl, methamphetamine HCl, methylphenidate HCl, pemoline, phendimetrazine tartrate, phentermine HCl, and sibutramine HCl monohydrate;

depigmenting agents: hydroquinone and monobenzone;

erectile dysfunction therapies: alprostadil and sildenafil (e.g., sildenafil citrate);

gastrointestinal agents: antispasmodics such as glycopyrolate; histamine receptor antagonists such as famotidine; and proton pump inhibitors such as esomeprazole, lansoprazole, omeprazole, pantoprazole, and rabeprazole sodium;

hematinic agents: cyanocobalamin (Vitamin B₁₂), ferric gluconate, ferric sulfate, ferrous gluconate, ferrous sulfate, and folic acid;

hemostatic drugs: desmopressin and desmopressin acetate;

hypocalcemics: calcitriol;

immunosuppressive agents: tacrolimus and sirolimus;

leukotriene inhibitors: montelukast sodium;

motion sickness drugs: promethazine HCl and scopolamine;

muscle relaxants: baclofen, cyclobenzaprine and cyclobenzaprine HCl, dantrolene, ritodrine HCl, tizanidine HCl, and tolterodine tartrate;

nicotine;

narcotic antagonists: naloxone, particularly naloxone hydrochloride; nutritional agents, including vitamins, minerals, essential amino acids and fatty acids, such as chromium picolinate, cyanocobalamin (vitamin B₁₂), ferric glyconate, ferric sulfate, ferrous glyconate, ferrous salt, ferrous sulfate, folic acid, vitamin C, zinc acetate, and zinc sulfate;

ophthalmic drugs: physostigmine sulfate;

oxytocics: dinoprostone;

peripheral vascular dilators: cyclandelate, isoxsuprine and papaverine;

prostaglandins: alprostadil, dinoprostone, and epoprostenol (e.g., epoprostenol sodium);

sedatives and hypnotics: diphenylhydramine HCl, melatonin, propofol, triazolam, zalepion, and zolpidem tartrate;

serotonin antagonists: alosetron HCl, altanserin tartrate, amesergide and ketanserin; ritanserin;

serotonin receptor agonists: 5HT receptor antagonists such as naratriptan HCl, rizatriptan benzoate, sumatriptan succinate, and zolmitriptan;

serotonin receptor antagonists: 5HT₃ (serotonin subtype 3 receptor) antagonists such as dolasetron, granisetron hydrochloride, ondansetron hydrochloride, and tropisetron;

steroids: betamethasone and augmented betamethasone, clobetasol propionate, desoximetasone, diflorasone diacetate, fluocinonide, flurandrenolide, fluticansone (e.g., fluticansonepropionate), halobetasol propionate, hydrocortisone, mometasone furonate, and prednicarbate;

thyroid preparations: antithyroid agents (e.g., methimazole), synthetic T3 compounds (e.g., liothyronine sodium), and synthetic T4 compounds (e.g., levothyroxine sodium);

tocolytic: salbutamol and ritodrine;

topoimerase inhibitors: topotecan and irinotecan;

Tourette's Syndrome agents: haloperidol and primozide; and

wart preparations: imiquimod.

Genetic material may also be delivered using the methods, formulations and transdermal systems of the invention, e.g., a nucleic acid, RNA, DNA, recombinant RNA, recombinant DNA, antisense RNA, antisense DNA, a ribooligonucleotide, a deooxyriboonucleotide, an antisense ribooligonucleotide, or an antisense deoxyriboooligonucleotide.

Particularly preferred systemically active agents that can be administered transdermally in conjunction with the present invention are as follows: buprenorphine, fentanyl, sufentanil, terbutaline, formoterol, albuterol, theophylline, estradiol, progesterone, scopolamine, enalapril, 1-carboxymethyl-3-1-carboxy-3-phenyl-(1S)-propylamino-2,3,4,5-tetrahydro-1H-(3S)1-benzazepine-2-one, 3-(5-amino-1-carboxy-1S-pentyl)amino-2,3,4,5-tetrahydro-2-oxo-3S-1H-1-benzazepine 1-acetic acid, 3-(1-ethoxycarbonyl-3-phenyl-(1S)-propylamino)-2,3,4,5-tetrahydro-2-oxo-(3S)-benzazepine-1-acetic acid monohydrochloride; nitroglycerin, triprolidine, tripelenamine, diphenhydramine, physostigmine, arecoline, and nicotine. Uncharged, nonionizable active agents are preferred, as are acid addition salts of basic drugs. Of the latter group, the hydrochloride salt is most preferred.

IV. Pharmaceutical Formulations

One embodiment of the invention is a composition for the enhanced delivery of a drug through a body surface, comprising a formulation of: (a) a therapeutically effective amount of the drug; (b) a pharmaceutically acceptable inorganic or organic base in an amount effective to provide a pH within the range of about 8.0-13.0 at the localized region of the body surface during administration of the drug and to enhance the flux of the drug through the body surface without causing damage thereto; and (c) a pharmaceutically acceptable carrier suitable for topical or transdermal drug administration. The formulation is typically, but not necessarily, an aqueous formulation. The pH is more preferably about 8.5-11.5, more preferably about 9.5-11.5 and most preferably about 10.0 to 11.5.

Accordingly, while the method of delivery of the active agent may vary, the method will typically involve application of a formulation or drug delivery system containing a pharmaceutically acceptable inorganic or organic base to a predetermined area of the skin or other tissue for a period of time sufficient to provide the desired local or systemic effect. The method may involve direct application of the composition as an ointment, gel, cream, or the like, or may involve use of a drug delivery device. In either case, water is preferably present in order for the hydroxide ions to be provided by the base, and thus enhance the flux of the active agent through the patient's body surface. Thus, such a formulation or drug reservoir may be aqueous, i.e., contain water, or may be nonaqueous and used in combination with an occlusive backing layer so that moisture evaporating from the body surface is maintained within the formulation or transdermal system during drug administration. In some cases, however, e.g., with an occlusive gel, a nonaqueous formulation may be used with or without an occlusive backing layer. Suitable formulations include ointments, creams, gels, lotions, solutions, pastes, and the like. Ointments, as is well known in the art of pharmaceutical formulation, are semisolid preparations that are typically based on petrolatum or other petroleum derivatives. The specific ointment foundation to be used, as will be appreciated by those skilled in the art, is one that will provide for optimum drug delivery, and, preferably, will provide for other desired characteristics as well, e.g., emolliency or the like. As with other carriers or vehicles, the ointment foundation should be inert, stable, nonirritating and nonsensitizing. As explained in Remington: The Science and Practice of Pharmacy, 20^{th} edition (Lippincott Williams & Wilkins, 2000), ointment foundations may be grouped in four classes: oleaginous, emulsifiable, emulsion, and water-soluble. Oleaginous ointment foundations include, for example, vegetable oils, fats obtained from animals, and semisolid hydrocarbons obtained from petroleum. Emulsifiable ointment foundations, also known as absorbent ointment foundations, contain little or no water and include, for example, hydroxystearin sulfate, anhydrous lanolin and hydrophilic petrolatum. Emulsion ointment foundations are either water-in-oil (W/O) emulsions or oil-in-water (O/W) emulsions, and include, for example, cetyl alcohol, glyceryl monostearate, lanolin and stearic acid. Preferred water-soluble ointment foundations are prepared from polyethylene glycols of varying molecular weight.

Creams, as also well known in the art, are viscous liquids or semisolid emulsions, either oil-in-water or water-in-oil. Cream foundations are water-washable, and contain an oil phase, an emulsifier and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant.

As will be appreciated by those working in the field of pharmaceutical formulation, gels are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the “carbomer” family of polymers, e.g., carboxypolyalkylenes that may be obtained commercially under the Carbopol® trademark. Also preferred are hydrophilic polymers such as polyethylene oxides, polyoxyethylene-polyoxypropylene copolymers and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Lotions, which are preferred for delivery of cosmetic agents, are preparations to be applied to the skin surface without friction, and are typically liquid or semiliquid preparations in which solid particles, including the active agent, are present in a water or alcohol base. Lotions are usually suspensions of solids, and preferably, for the present purpose, comprise a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

Solutions are homogeneous mixtures prepared by dissolving one or more chemical substances (solute) in another liquid such that the molecules of the dissolved substance are dispersed among those of the solvent. The solution may contain other pharmaceutically acceptable chemicals to buffer, stabilize or preserve the solute. Commonly used examples of solvents used in preparing solutions are ethanol, water, propylene glycol or any other pharmaceutically acceptable vehicle.

Pastes are semisolid dosage forms in which the active agent is suspended in a suitable foundation. Depending on the nature of the foundation, pastes are divided between fatty pastes or those made from single-phase, aqueous gels. The foundation in a fatty paste is generally petrolatum or hydrophilic petrolatum or the like. The pastes made from single-phase aqueous gels generally incorporate carboxymethylcellulose or the like as the foundation.

Formulations may also be prepared with liposomes, micelles, and microspheres. Liposomes are microscopic vesicles having a lipid wall comprising a lipid bilayer, and can be used as drug delivery systems herein as well. Generally, liposome formulations are preferred for poorly soluble or insoluble pharmaceutical agents. Liposomal preparations for use in the instant invention include cationic (positively charged), anionic (negatively charged) and neutral preparations. Cationic liposomes are readily available. For example, N-[1-2,3-dioleyloxy)propyl]-N,N,N-triethylammonium liposomes are available under the tradename Lipofectin® (GIBCO BRL, Grand Island, N.Y.). Anionic and neutral liposomes are readily available as well, e.g., from Avanti Polar Lipids (Birmingham, Ala.), or can be easily prepared using readily available materials. Such materials include phosphatidyl choline, cholesterol, phosphatidyl ethanolamine, dioleoylphosphatidyl choline, dioleoylphosphatidyl glycerol, dioleoylphoshatidyl ethanolamine, among others. These materials can also be mixed with N-[1-2,3-dioleyloxy)propyl]-N,N,N-triethylammonium (DOTMA) in appropriate ratios. Methods for making liposomes using these materials are well known in the art.

Micelles are known in the art and are comprised of surfactant molecules arranged so that their polar headgroups form an outer spherical shell, while the hydrophobic, hydrocarbon chains are oriented towards the center of the sphere, forming a core. Micelles form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethyl-ammonium chloride, dodecylammonium chloride, polyoxyl 8 dodecyl ether, polyoxyl 12 dodecyl ether, nonoxynol 10 and nonoxynol 30. Micelle formulations can be used in conjunction with the present invention either by incorporation into the reservoir of a topical or transdermal delivery system, or into a formulation to be applied to the body surface.

Microspheres, similarly, may be incorporated into the present formulations and drug delivery systems. Like liposomes and micelles, microspheres essentially encapsulate a drug or drug-containing formulation. They are generally, although not necessarily, formed from lipids, preferably charged lipids such as phospholipids. Preparation of lipidic microspheres is well known in the art and described in the pertinent texts and literature.

Various additives, known to those skilled in the art, may be included in the topical formulations. For example, solvents, including relatively small amounts of alcohol, may be used to solubilize certain drug substances. Other optional additives include opacifiers, antioxidants, fragrance, colorant, gelling agents, thickening agents, stabilizers, surfactants and the like. Other agents may also be added, such as antimicrobial agents, to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof.

For those drugs having an unusually low rate of permeation through the skin or mucosal tissue, it may be desirable to include a second permeation enhancer in the formulation in addition to the inorganic or organic base enhancer, although in a preferred embodiment the base enhancer is administered without any other permeation enhancers. Any other enhancers should, like the base enhancer, minimize the possibility of skin damage, irritation, and systemic toxicity. Examples of classes of suitable secondary enhancers (or “co-enhancers”) include, but are not limited to, fatty acids, both saturated and unsaturated; fatty alcohols; bile acids; nonionic surfactants, including esters of fatty acids, fatty (long-chain alkyl or alkenyl) esters of monohydric alcohols, diols, and polyols, diols and polyols that are both esterified with a fatty acid and substituted with a polyoxyalkylene, polyoxyalkylene fatty acid esters, polyoxyalkylene fatty ethers, polyoxyalkylene fatty ethers, and polyglyceryl fatty acid esters; amines; amides; N-alkyl-azacycloalkanones and N-alkyl-azacycloalkenones; hydrocarbon solvents; terpenes; lower alkyl esters; cyclodextrin enhancers; nitrogen-containing heterocycles; sulfoxides; and urea and its derivatives.

Specific examples of suitable co-enhancers include ethers such as diethylene glycol monoethyl ether (available commercially as Transcutol®, Gattefosse SA) and diethylene glycol monomethyl ether; surfactants such as sodium laurate, sodium lauryl sulfate, cetyltrimethylammonium bromide, benzalkonium chloride, Poloxamer (231, 182, 184), Tween (20, 40, 60, 80) and lecithin; alcohols such as ethanol, propanol, octanol, benzyl alcohol, and the like; fatty acids such as lauric acid, oleic acid and valeric acid; fatty acid esters such as isopropyl myristate, isopropyl palmitate, methylpropionate, and ethyl oleate; polyols and esters thereof such as polyethylene glycol, and polyethylene glycol monolaurate; amides and other nitrogenous compounds such as urea, dimethylacetamide, dimethylformamide, 2-pyrrolidone, 1-methyl-2-pyrrolidone, ethanolamine, diethanolamine and triethanolamine; terpenes; alkanones; and organic acids, particularly citric acid and succinic acid. Azone® and sulfoxides such as dimethylsulfoxide and decylmethylsulfoxide may also be used, but are less preferred. Percutaneous Penetration Enhancers, eds. Smith et al. (CRC Press, 1995) provides an excellent overview of the field and further information concerning possible secondary enhancers for use in conjunction with the present invention.

The formulation may also contain irritation-mitigating additives to minimize or eliminate the possibility of skin irritation or skin damage resulting from the drug, the base enhancer, or other components of the formulation. Suitable irritation-mitigating additives include, for example: α-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine. The irritant-mitigating additive, if present, may be incorporated into the formulation at a concentration effective to mitigate irritation or skin damage, typically representing not more than about 20 wt %, more typically not more than about 5 wt %, of the formulation.

The concentration of the active agent in the formulation will typically depend upon a variety of factors, including the disease or condition to be treated, the nature and activity of the active agent, the desired effect, possible adverse reactions, the ability and speed of the active agent to reach its intended target, and other factors within the particular knowledge of the patient and physician. Preferred formulations will typically contain on the order of about 0.5-50 wt %, preferably about 5-30 wt %, active agent.

V. Drug Delivery Systems

An alternative and preferred method involves the use of a drug delivery system, e.g., a topical or transdermal “patch,” wherein the active agent is contained within a laminated structure that is to be affixed to the skin. In such a structure, the drug composition is contained in a layer, or “reservoir,” underlying an upper backing layer that serves as the outer surface of the device during use. The laminated structure may contain a single reservoir, or it may contain multiple reservoirs.

Accordingly, another embodiment of the invention is a system for the enhanced topical or transdermal administration of a drug, comprising: (a) at least one drug reservoir containing the drug and a pharmaceutically acceptable inorganic or organic base in an amount effective to enhance the flux of the drug through the body surface without causing damage thereto; (b) a means for maintaining the system in drug and base transmitting relationship to the body surface and forming a body surface-system interface; and (c) a backing layer that serves as the outer surface of the device during use, wherein the base is effective to provide a pH within the range of about 8.0-13.0 at the body surface-system interface during administration of the drug. The pH is more preferably about 8.5-11.5, more preferably about 9.5-11.5 and most preferably about 10.0 to 11.5.

In one embodiment, the drug reservoir comprises a polymeric matrix of a pharmaceutically acceptable adhesive material that serves to affix the system to the skin during drug delivery; typically, the adhesive material is a pressure-sensitive adhesive (PSA) that is suitable for long-term skin contact, and which should be physically and chemically compatible with the active agent, inorganic or organic base, and any carriers, vehicles or other additives that are present. Examples of suitable adhesive materials include, but are not limited to, the following: polyethylenes; polysiloxanes; polyisobutylenes; polyacrylates; polyacrylamides; polyurethanes; plasticized ethylene-vinyl acetate copolymers; and tacky rubbers such as polyisobutene, polybutadiene, polystyrene-isoprene copolymers, polystyrene-butadiene copolymers, and neoprene (polychloroprene). Preferred adhesives are polyisobutylenes.

The backing layer functions as the primary structural element of the transdermal system and provides the device with flexibility and, preferably, occlusivity. The material used for the backing layer should be inert and incapable of absorbing the drug, the base enhancer, or other components of the formulation contained within the device. The backing is preferably comprised of a flexible elastomeric material that serves as a protective covering to prevent loss of drug and/or vehicle via transmission through the upper surface of the patch, and will preferably impart a degree of occlusivity to the system, such that the area of the body surface covered by the patch becomes hydrated during use. The material used for the backing layer should permit the device to follow the contours of the skin and be worn comfortably on areas of skin such as at joints or other points of flexure, that are normally subjected to mechanical strain with little or no likelihood of the device disengaging from the skin due to differences in the flexibility or resiliency of the skin and the device. The materials used as the backing layer are either occlusive or permeable, as noted above, although occlusive backings are preferred, and are generally derived from synthetic polymers (e.g., polyester, polyethylene, polypropylene, polyurethane, polyvinylidine chloride, and polyether amide), natural polymers (e.g., cellulosic materials), or macroporous woven and nonwoven materials.

During storage and prior to use, the laminated structure preferably includes a release liner. Immediately prior to use, this layer is removed from the device so that the system may be affixed to the skin. The release liner should be made from a drug/vehicle impermeable material, and is a disposable element, which serves only to protect the device prior to application. Typically, the release liner is formed from a material impermeable to the pharmacologically active agent and the base enhancer, and is easily stripped from the transdermal patch prior to use.

In an alternative embodiment, the drug-containing reservoir and skin contact adhesive are present as separate and distinct layers, with the adhesive underlying the reservoir. In such a case, the reservoir may be a polymeric matrix as described above. Alternatively, the reservoir may be comprised of a liquid or semisolid formulation contained in a closed compartment or pouch, or it may be a hydrogel reservoir, or may take some other form. Hydrogel reservoirs are particularly preferred herein. As will be appreciated by those skilled in the art, hydrogels are macromolecular networks that absorb water and thus swell but do not dissolve in water. That is, hydrogels contain hydrophilic functional groups that provide for water absorption, but the hydrogels are comprised of crosslinked polymers that give rise to aqueous insolubility. Generally, then, hydrogels are comprised of crosslinked hydrophilic polymers such as a polyurethane, a polyvinyl alcohol, a polyacrylic acid, a polyoxyethylene, a polyvinylpyrrolidone, a poly(hydroxyethyl methacrylate)(poly(HEMA)), or a copolymer or mixture thereof. Particularly preferred hydrophilic polymers are copolymers of HEMA and polyvinylpyrrolidone.

Additional layers, e.g., intermediate fabric layers and/or rate-controlling membranes, may also be present in any of these drug delivery systems. Fabric layers may be used to facilitate fabrication of the device, while a rate-controlling membrane may be used to control the rate at which a component permeates out of the device. The component may be a drug, a base enhancer, an additional enhancer, or some other component contained in the drug delivery system.

A rate-controlling membrane, if present, will be included in the system on the skin side of one or more of the drug reservoirs. The material used to form such a membrane is selected so as to limit the flux of one or more components contained in the drug formulation. Representative materials useful for forming rate-controlling membranes include polyolefins such as polyethylene and polypropylene, polyamides, polyesters, ethylene-ethacrylate copolymer, ethylene-vinyl acetate copolymer, ethylene-vinyl methylacetate copolymer, ethylene-vinyl ethylacetate copolymer, ethylene-vinyl propylacetate copolymer, polyisoprene, polyacrylonitrile, ethylene-propylene copolymer, and the like.

Generally, the underlying surface of the transdermal device, i.e., the skin contact area, has an area in the range of about 5-200 cm², preferably 5-100 cm², more preferably 20-60 cm². That area will vary, of course, with the amount of drug to be delivered and the flux of the drug through the body surface. Larger patches can be used to accommodate larger quantities of drug, while smaller patches can be used for smaller quantities of drug and/or drugs that exhibit a relatively high permeation rate.

Such drug delivery systems may be fabricated using conventional coating and laminating techniques known in the art. For example, adhesive matrix systems can be prepared by casting a fluid admixture of adhesive, drug and vehicle onto the backing layer, followed by lamination of the release liner. Similarly, the adhesive mixture may be cast onto the release liner, followed by lamination of the backing layer. Alternatively, the drug reservoir may be prepared in the absence of drug or excipient, and then loaded by soaking in a drug/vehicle mixture. In general, transdermal systems of the invention are fabricated by solvent evaporation, film casting, melt extrusion, thin film lamination, die cutting, or the like. The inorganic or organic base permeation enhancer will generally be incorporated into the device during patch manufacture rather than subsequent to preparation of the device. Thus, for acid addition salts of basic drugs (e.g., hydrochloride salts of amine drugs), the enhancer will neutralize the drug during manufacture of the drug delivery system, resulting in a final drug delivery system in which the drug is present in nonionized, neutral form along with an excess of base to serve as a permeation enhancer. For nonionized acidic drugs, the base will neutralize such drugs by converting them to the ionized drug in salt form.

In a preferred delivery system, an adhesive overlayer that also serves as a backing for the delivery system is used to better secure the patch to the body surface. This overlayer is sized such that it extends beyond the drug reservoir so that adhesive on the overlayer comes into contact with the body surface. The overlayer is useful because the adhesive/drug reservoir layer may lose its adhesion a few hours after application due to hydration. By incorporating an adhesive overlayer, the delivery system will remain in place for the required period of time.

Other types and configurations of transdermal drug delivery systems may also be used in conjunction with the method of the present invention, as will be appreciated by those skilled in the art of transdermal drug delivery. See, for example, Ghosh, Transdermal and Topical Drug Delivery Systems (Interpharm Press, 1997), particularly Chapters 2 and 8.

As with the topically applied formulations of the invention, the drug and enhancer composition contained within the drug reservoir(s) of these laminated systems may comprise a number of additional components. In some cases, the drug and enhancer may be delivered neat, i.e., in the absence of additional liquid. In most cases, however, the drug will be dissolved, dispersed or suspended in a suitable pharmaceutically acceptable vehicle, typically a solvent or gel. Other components that may be present include preservatives, stabilizers, surfactants, solubilizers, additional enhancers, and the like.

The invention accordingly provides a novel and highly effective means for increasing the flux of an active agent through the body surface (skin or mucosal tissue) of a human or animal. The base enhancers discussed herein, employed in specific amounts relative to a formulation or drug reservoir, may be used as permeation enhancers with a wide variety of drugs and drug types, including free acids, free bases, acid addition salts of basic drugs, basic addition salts of acidic drugs, nonionizable drugs, peptides and proteins. Surprisingly, the increase in permeation is not accompanied by any noticeable tissue damage, irritation, or sensitization. The invention thus represents an important advance in the field of drug delivery.

It is to be understood that while the invention has been described in conjunction with the preferred specific embodiments thereof, the foregoing description is intended to illustrate and not limit the scope of the invention. Other aspects, advantages and modifications will be apparent to those skilled in the art to which the invention pertains. Furthermore, the practice of the present invention will employ, unless otherwise indicated, conventional techniques of drug formulation, particularly topical and transdermal drug formulation, which are within the skill of the art. Such techniques are fully explained in the literature. See Remington: The Science and Practice of Pharmacy, cited supra, as well as Goodman & Gilman's The Pharmacological Basis of Therapeutics, 10^{th} Ed.(2001).

The following examples are put forth so as to provide those of ordinary skill in the art with a complete disclosure and description of how to practice the methods as well as make and use the compositions of the invention, and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C. and pressure is at or near atmospheric. The following abbreviations will be used in accordance with the definitions set out below.

### EXAMPLES

ABBREVIATIONS

| | |
|---|---|
| DI | Deionized |
| HPMC | Hydroxypropylmethylcellulose |
| HPMCP | Hydroxypropylmethylcellulose phthalate |
| PG | Propylene glycol |
| PIB | Polyisobutylene |

### Methods

### Preparation of Round Disc Samples

Each formulation was coated onto a release liner and dried in an oven at 55° C. for two hours to remove water and other solvents. The dried drug-in-adhesive/release liner film was laminated to a backing film. The backing/drug-in-adhesive/release liner laminate was then cut into round discs with a diameter of 11/16 inch.

### Measurement of Permeation of Drugs Through Human Cadaver Skin

The in vitro permeation of drugs through human cadaver skin was performed using Franz-type diffusion cells with a diffusion area of 1 cm². The volume of receiver solution was 8 ml. Human cadaver skin was cut to a proper size and placed on a flat surface with the stratum corneum side facing up. The release liner was peeled away from the disc laminate. The backing/drug-in-adhesive film was placed and pressed on the skin with the adhesive side facing the stratum corneum. The skin/adhesive/backing laminate was clamped between the donor and receiver chambers of the diffusion cell with the skin side facing the receiver solution.

### Measurement of pH

The pH of the patches was measured using the following procedures. A 2.5 cm² circular patch was punched out. Ten ml purified water was pipetted into a glass vial, and a stir bar was added. The liner was removed from the patch and placed in the vial along with the patch. The vial was then placed on a stir plate and the water/patch/liner mixture was stirred for 5 minutes, at which point the liner was removed from the vial and discarded. The vial was again placed on a stir plate and stirring continued for an additional 18 hours. After 18 hours, the stir bar was removed from the vial and the pH of the solution determined using a calibrated pH meter.

### Example 1

An in vitro skin permeation study was conducted using three estradiol transdermal systems, designated Est-1, Est-2, and Est-3, the compositions of which are set forth in Table 1. Round disc samples were prepared as described in the Methods section. The theoretical percent weight for each ingredient after drying (calculated assuming all volatile ingredients were completely removed during drying) is set forth in Table 2.

TABLE 1
Component Weight and Weight Percent Based on Total Solution
Weight

| | Est-1 | Est-2 | Est-3 |
|---|---|---|---|
| | g (wt %) | g (wt %) | g (wt %) |
| Estradiol | 0.0313 (0.5) | 0.0322 (0.5) | 0.0308 (0.5) |
| NaOH | 0 | 0.0155 (0.3) | 0.025 (0.4) |
| DI water | 0 | 0.4155 (6.9) | 0.425 (7.0) |
| PIB adhesive (30% solid) | 4 (66.3) | 4 (66.0) | 4 (65.8) |
| Methylal | 1.8 (29.8) | 1.4 (23.1) | 1.4 (23.0) |
| Ethanol | 0.2 (3.3) | 0.2 (3.3) | 0.2 (3.3) |

TABLE 2
Component Weight and Weight Percent Based on Dried Film Weight

| | Est-1 | Est-2 | Est-3 |
|---|---|---|---|
| | g (wt %) | g (wt %) | g (wt %) |
| Estradiol | 0.0313 (2.5) | 0.0322 (2.6) | 0.0308 (2.5) |
| NaOH | 0 | 0.0155 (1.2) | 0.025 (2.0) |
| PIB adhesive | 1.2 (97.5) | 1.2 (96.2) | 1.2 (95.6) |

The pH of the patches was measured as described in the Methods section. The pH of the estradiol patch measured using these procedures increased from 7.22 to 8.90 when the calculated NaOH concentration in the dried patch was increased from 0% to 2.0%. The measured pHs for the estradiol transdermal systems are listed below.

TABLE 3
pH

| Est-1 | Est-2 | Est-3 |
|---|---|---|
| 7.22 | 8.75 | 8.90 |

The in vitro permeation of estradiol through human cadaver skin from these discs was measured as described in the Methods section. Three diffusion cells were used for each formulation. The cells were filled with a 10% ethanol/90% water solution. The receiver solution was completely withdrawn and replaced with fresh ethanol/water solution at each time point. The samples taken were analyzed by HPLC to determine the concentration of estradiol in the receiver solution. The cumulative amount of estradiol that permeated through the human cadaver skin was calculated using the measured estradiol concentrations in the receiver solutions. The cumulative amount of estradiol that permeated across human cadaver skin at 24 hours increased from 0.22 μg/cm² to 7.01 μg/cm² when the calculated NaOH concentration in the dried patch was increased from 0% to 2.0%. The cumulative amount of estradiol that permeated across human cadaver skin at 24 hours from the system containing 1.2% NaOH (Est-2) was 4.55 μg/cm², which was about 20 times higher than that from the formulation without NaOH (0.22 μg/cm², Est-1).

Therefore, the formulation of Est-2 provided about 20-fold more estradiol flux than in the absence of NaOH (Est-1), while the highest pH formulation evaluated, Est-3, provided about 31-fold more flux than in the absence of NaOH.

### Example 2

An in vitro skin permeation study was conducted using four ketoprofen transdermal systems, designated Keto-1, Keto-2, Keto-3 and Keto-4, the compositions of which are set forth in Table 4. Round disc samples were prepared as described in the Methods section. The theoretical percent weight for each ingredient after drying (calculated assuming all volatile ingredients were completely removed during drying) is set forth in Table 5.

TABLE 4
Component Weight and Weight Percent Based on Total Solution
Weight

| | Keto-1 | Keto-2 | Keto-3 | Keto-4 |
|---|---|---|---|---|
| | g (wt %) | g (wt %) | g (wt %) | g (wt %) |
| Ketoprofen | 1.2 (16.7) | 1.2 (15.8) | 1.2 (15.7) | 1.2 (15.7) |
| NaOH | 0 | 0.19 (2.5) | 0.215 (2.8) | 0.225 (2.9) |
| DI water | 0 | 0.19 (2.5) | 0.215 (2.8) | 0.225 (2.9) |
| PIB adhesive | 4 (55.6) | 4 (52.8) | 4 (52.4) | 4 (52.3) |
| (30% solid) | | | | |
| Methylal | 2 (27.8) | 2 (26.4) | 2 (26.2) | 2 (26.1) |

TABLE 5
Weight and Theoretical Weight Percent Based on Dried Film Weight

| | Keto-1 | Keto-2 | Keto-3 | Keto-4 |
|---|---|---|---|---|
| | g (wt %) | g (wt %) | g (wt %) | g (wt %) |
| Ketoprofen | 1.2 (50) | 1.2 (45.9) | 1.2 (45.9) | 1.2 (45.7) |
| NaOH | 0 | 0.19 (7.3) | 0.215 (8.2) | 0.225 (8.6) |
| PIB adhesive | 1.2 (50) | 1.2 (46.3) | 1.2 (45.9) | 1.2 (45.7) |

Since ketoprofen is a free acid, it reacts with NaOH. The concentration of NaOH in the system after the reaction is completed depends on the amount of ketoprofen added. The remaining NaOH concentration after the reaction is completed is defined as “excess NaOH concentration,” which is defined by the following equation.

[NaOH_{excess}]=[NaOHₜₒₜₐₗ]−[NaOH_{needed for neutralization}]

The excess NaOH concentrations for the four ketoprofen systems were calculated, and the pH of each patch was measured as described in the Methods section. The pH increased from 8.60 to 10.57 when the calculated excess NaOH concentration in the dried patch was increased from 0.05% to 1.38%.

TABLE 6
Excess NaOH Concentration (wt %) and pH

| | Keto-1 | Keto-2 | Keto-3 | Keto-4 |
|---|---|---|---|---|
| Excess NaOH | — | 0.05% | 1.00% | 1.38% |
| Concentration | | | | |
| pH | 3.68 | 8.60 | 10.10 | 10.57 |

The in vitro permeation of ketoprofen through human cadaver skin from these discs was measured as described in the Methods section. Five diffusion cells were used for each formulation. Normal saline was used as the receiver solution. The volume of receiver solution was 8 ml. The entire receiver solution was collected and replaced with fresh saline at each time point. The receiver solution collected was analyzed by HPLC to determine the concentration of ketoprofen. The cumulative amount of ketoprofen that permeated across the human cadaver skin was calculated using the measured ketoprofen concentrations in the receiver solutions, which were plotted versus time and are described below.

Even though patch Keto-2 contained 7.3% NaOH, the cumulative amount of ketoprofen that permeated across the human cadaver skin at 24 hours (61.7 μg/cm²) was only slightly higher than that from the formulation without NaOH (Keto-l, 35.2 μg/cm²). This may be due to the consumption of NaOH by the reaction between NaOH and ketoprofen, which reduced the NaOH concentration to only 0.05% as the excess NaOH concentration. This result indicated that the permeation of ketoprofen could be enhanced with an excess NaOH concentration as low as 0.05%.

The cumulative amount of ketoprofen that permeated across human cadaver skin at 24 hours increased from 61.7 μg/cm² to 402.7 μg/cm² when the calculated excess NaOH concentration in the dried patch was increased from 0.05% to 1.38% (Keto-4), i.e., up to about 7-fold more flux was obtained than in the absence of NaOH. The cumulative amount of ketoprofen that permeated across human cadaver skin at 24 hours from the formulation with an excess NaOH concentration of 1.00% (Keto-3, 315.8 μg/cm²) is about 5 times higher than that from the formulation with an excess NaOH concentration of 0.05% (Keto-2, 61.7 μg/cm²).

### Example 3

An in vitro skin permeation study was conducted using four phenylpropanolamine hydrochloride (PPA-HCl) transdermal systems, designated PPA-1, PPA-2, PPA-3, and PPA-4, the compositions of which are set forth in Table 7. Round disc samples were prepared as described in the Methods section. The theoretical percent weight for each ingredient after drying (calculated assuming all volatile ingredients were completely removed during drying) is set forth in Table 8.

TABLE 7
Component Weight and Weight Percent Based on Total Solution
Weight

| | PPA-1 | PPA-2 | PPA-3 | PPA-4 |
|---|---|---|---|---|
| | g (wt %) | g (wt %) | g (wt %) | g (wt %) |
| PPA-HCl | 0.75 (8.5) | 0.75 (8.2) | 0.75 (8.1) | 0.75 (8.1) |
| NaOH | 0 | 0.165 (1.8) | 0.195 (2.1) | 0.23 (2.5) |
| DI water | 1.1 (12.4) | 1.265 (13.8) | 1.295 (14.0) | 1.33 (14.3) |
| PG | 0.5 (5.6) | 0.5 (5.4) | 0.5 (5.4) | 0.5 (5.4) |
| Methylal | 1 (11.3) | 1 (10.9) | 1 (10.8) | 1 (10.7) |
| Heptane | 1.5 (16.9) | 1.5 (16.3) | 1.5 (16.2) | 1.5 (16.1) |
| PIB adhesive | 4 (45.2) | 4 (43.6) | 4 (43.3) | 4 (43.0) |
| (30% solid) | | | | |

TABLE 8
Weight and Theoretical Weight Percent Based on Dried Film Weight

| | PPA-1 | PPA-2 | PPA-3 | PPA-4 |
|---|---|---|---|---|
| | g (wt %) | g (wt %) | g (wt %) | g (wt %) |
| PPA-HCl | 0.75 (30.6) | 0.75 (28.7) | 0.75 (28.4) | 0.75 (28.0) |
| NaOH | 0 | 0.165 (6.3) | 0.195 (7.4) | 0.23 (8.6) |
| PIB adhesive | 1.2 (49.0) | 1.2 (45.9) | 1.2 (45.4) | 1.2 (44.8) |
| PG | 0.5 (20.4) | 0.5 (19.1) | 0.5 (18.9) | 0.5 (18.7) |

Since PPA-HCl is an acid addition salt of a free base, it reacts with NaOH. The concentration of NaOH in the system after the reaction is completed depends on the amount of PPA-HCl added. The remaining NaOH concentration after the reaction is completed is defined as the excess NaOH concentration, and was calculated as described in Example 2. The pH was measured as described in the Methods section. The pH of the PPA-HCl patch increased from 10.08 to 10.88 when the calculated excess NaOH concentration in the dried patch was increased from 0.20% to 2.62%, while the pH of the patch without NaOH was 7.33. Skin irritation could be related to the pH of the patch, which depends on the excess NaOH concentration.

TABLE 9
Excess NaOH Concentration (wt %) and pH

| | PPA-1 | PPA-2 | PPA-3 | PPA-4 |
|---|---|---|---|---|
| Excess NaOH | — | 0.20% | 1.33% | 2.62% |
| Concentration | | | | |
| pH | 7.33 | 10.08 | 10.16 | 10.88 |

The in vitro permeation of PPA-HCl through human cadaver skin from these discs was measured as described in the Methods section. Three diffusion cells were used for each formulation. The cells were filled with DI water. The receiver solution was completely withdrawn and replaced with fresh DI water at each time point. The samples taken were analyzed by an HPLC for the concentration of PPA-HCl in the receiver solution. The cumulative amount of PPA-HCl that permeated across the human cadaver skin was calculated using the measured PPA-HCl concentrations in the receiver solutions, which were plotted versus time and are described below.

Even though patch PPA-2 contained 6.3% NaOH, the cumulative amount of PPA-HCl that permeated across the human cadaver skin at 24 hours from this formulation (1.35 mg/cm²) was only slightly higher than that from the formulation without NaOH (PPA-1, 0.56 mg/cm²). This may be due to the consumption of NaOH by the reaction between NaOH and PPA-HCl, which reduced the NaOH concentration to only 0.20% as the excess NaOH concentration. This result indicated that the permeation of PPA-HCl could be enhanced with an excess NaOH concentration as low as 0.20%. The cumulative amount of PPA-HCl across human cadaver skin at 24 hours increased from 1.35 mg/cm² to 5.99 mg/cm² when the calculated excess NaOH concentration in the dried patch was increased from 0.20% to 2.62% (PPA-4), i.e., up to about 4-fold more flux was obtained than in the absence of NaOH. The cumulative amount of PPA-HCl across human cadaver skin at 24 hours from the formulation with an excess NaOH concentration of 1.33% (PPA-3, 5.2 mg/cm²) is about 4 times higher than that from the formulation with an excess NaOH concentration of 0.20% (PPA-2, 1.35 mg/cm²).

### Example 4

A human skin irritation study was performed using seven transdermal systems, which are listed below:

Keto-5 (containing no ketoprofen, no NaOH)

Keto-6 (containing ketoprofen, no NaOH)

Keto-7

Keto-8

Keto-9

Keto-10

Control (containing petrolatum)

The Control was an occlusive chamber (Hilltop, Cincinnati, Ohio) containing petrolatum held in place with paper tape. The following procedures were used to prepare the systems with the exception of the system containing petrolatum. The formulations used to prepare these systems are listed in Table 10, which include weight and weight percent of each component in the formulations. Round disc samples were prepared as described in the Methods section, except that the discs has a diameter of ½ inch. The theoretical percent weight for each ingredient after drying is listed in Table 11, which was calculated assuming all the volatile ingredients were completely removed during drying.

TABLE 10
Component Weight and Weight Percent Based on Total Solution
Weight

| | Keto-7 | Keto-8 | Keto-9 | Keto-10 |
|---|---|---|---|---|
| | g (wt %) | g (wt %) | g (wt %) | g (wt %) |
| Ketoprofen | 2.4 (14.0) | 2.4 (14.0) | 2.4 (13.9) | 2.4 (13.8) |
| NaOH | 0.6 (3.5) | 0.65 (3.8) | 0.69 (4.0) | 0.73 (4.2) |
| DI water | 0.6 (3.5) | 0.65 (3.8) | 0.69 (4.0) | 0.73 (4.2) |
| Tetraglycol | 0.5 (2.9) | 0.5 (2.9) | 0.5 (2.9) | 0.5 (2.9) |
| Isopropylmyristate | 0.4 (2.3) | 0.4 (2.3) | 0.4 (2.3) | 0.4 (2.3) |
| Methyl salicylates | 0.6 (3.5) | 0.6 (3.5) | 0.6 (3.5) | 0.6 (3.5) |
| Methylal | 4 (23.4) | 4 (23.3) | 4 (23.3) | 4 (23.0) |
| PIB adhesive | 8 (46.8) | 8 (46.5) | 8 (46.3) | 8 (46.1) |
| (30% solid) | | | | |

TABLE 11
Weight and Theoretical Weight Percent Based on Dried Film Weight

| | Keto-7 | Keto-8 | Keto-9 | Keto-10 |
|---|---|---|---|---|
| | g (wt %) | g (wt %) | g (wt %) | g (wt %) |
| Ketoprofen | 2.4 (34.8) | 2.4 (34.5) | 2.4 (34.3) | 2.4 (34.1) |
| NaOH | 0.6 (8.7) | 0.65 (9.4) | 0.69 (9.9) | 0.73 (10.4) |
| PIB adhesive | 2.4 (34.0) | 2.4 (34.5) | 2.4 (34.3) | 2.4 (34.1) |
| Tetraglycol | 0.5 (7.2) | 0.5 (7.2) | 0.5 (7.2) | 0.5 (7.1) |
| Isopropylmyristate | 0.4 (5.8) | 0.4 (5.8) | 0.4 (5.7) | 0.4 (5.7) |
| Methyl salicylates | 0.6 (8.7) | 0.6 (8.6) | 0.6 (8.6) | 0.6 (8.5) |

Ten healthy human subjects were included in the skin irritation study. Each subject wore seven patches listed above on the arms for 24 hours. An adhesive film with a diameter of ⅞ inch was applied over each system on the skin except the petrolatum patch to secure the system and to make the system occlusive for 24 hours. After 24 hours, the patches were removed and the skin was scored on a 0-4 scale. The scoring scale employed is listed below. The skin was scored again at 48 hours.

| | |
|---|---|
| 0 = negative | 2 = erythema and induration |
| + = equivocal reaction (0.5) | 3 = erythema, induration and vesicles |
| 1 = erythema | 4 = bullae |

The in vitro permeation of ketoprofen through human cadaver skin from formulations Keto-7, Keto-8, Keto-9, and Keto-10, was measured as described in the Methods section. Three diffusion cells were used for each formulation.

Normal saline was used as the receiver solution. The receiver solution was collected at 24 hours and analyzed by an HPLC for the concentration of ketoprofen. The cumulative amount of ketoprofen that permeated across the human cadaver skin at 24 hours, was calculated using the measured ketoprofen concentrations in the receiver solutions. The excess NaOH concentrations for these four ketoprofen systems, were calculated as described in Example 2, and the pH of the patches was measured as described in the Methods section.

TABLE 12
Excess NaOH Concentration (wt %), Cumulative Permeation Amount
(mg/cm²), pH

| | Keto-7 | Keto-8 | Keto-9 | Keto-10 |
|---|---|---|---|---|
| Excess NaOH | 3.22% | 3.92% | 4.47% | 5.01% |
| Concentration | | | | |
| Cumulative | 0.17 | 0.34 | 0.54 | 1.52 |
| permeation amount | | | | |
| pH | 10.06 | 10.81 | 11.04 | 11.18 |

The cumulative amount of ketoprofen that permeated across the human cadaver skin at 24 hours increased from 0.17 mg/cm² to 1.52 mg/cm² when the calculated excess NaOH concentration in the dried patch was increased from 3.22% to 5.01%. The excess NaOH concentration and the cumulative amount of ketoprofen across skin at 24 hours and the patch pH for Keto-8 was 0.34 mg/cm² and 10.81 respectively, which was about the same as those for Keto-3 shown in Example 2 (0.32 mg/cm², pH=0.10). However, the excess NaOH concentration for Keto-8 (3.92%) was higher than that for Keto-3 (1.00%), which may be due to the consumption of NaOH through reactions between NaOH and components other than ketoprofen in the Keto-8 formulation.

The irritation scores obtained indicate that irritation from this patch was insignificant.

The formulation of Keto-8 provided up to 2-fold more ketoprofen flux than the lowest pH formulation evaluated (Keto-7). The formulation of Keto-9 provided up to 3-fold more flux, while the highest pH formulation evaluated, Keto-10, provided up to 9-fold more flux than in the absence of NaOH.

### Example 5

An in vitro skin permeation study was conducted using four ibuprofen transdermal gels, designated Ibu-1, Ibu-2, Ibu-3, and Ibu-84, the compositions of which are set forth in Table 13.

TABLE 13
Component Weight and Weight Percent Based on Total Solution
Weight

| | Ibu-1 | Ibu-2 | Ibu-3 | Ibu-4 |
|---|---|---|---|---|
| | g (wt %) | g (wt %) | g (wt %) | g (wt %) |
| Ibuprofen | 0.6 (36.8) | 0.6 (32.3) | 0.6 (31.6) | 0.6 (31.1) |
| NaOH | 0 | 0.115 (6.2) | 0.135 (7.1) | 0.15 (7.8) |
| Ethanol | 0.4 (24.5) | 0.4 (21.5) | 0.4 (21.1) | 0.4 (20.7) |
| DI water | 0.6 (36.8) | 0.715 (38.4) | 0.735 (38.7) | 0.75 (38.9) |
| HPMCP | 0.03 (1.8) | 0.03 (1.6) | 0.03 (1.6) | 0.03 (1.6) |

The excess NaOH concentrations for these four ibuprofen gels, were calculated as described in Example 2, and the pH of the gels was directly measured using a pH meter. The pH of the ibuprofen gel (determined using the procedures of the previous examples) increased from 6.58 to 12.22 when the calculated excess NaOH concentration in the gel was increased from 0% to 1.74%. The skin irritation could be related to the pH of the gel, which depends on the excess NaOH concentration.

TABLE 14
Excess NaOH Concentration (wt %) and pH

| | Ibu-1 | Ibu-2 | Ibu-3 | Ibu-4 |
|---|---|---|---|---|
| Excess NaOH | — | 0% | 0.98% | 1.74% |
| Concentration | | | | |
| pH | 4.57 | 6.58 | 11.83 | 12.22 |

The in vitro permeation of ibuprofen through human cadaver skin from these gels was measured in a slightly different manner than as described in the Methods section. Three diffusion cells were used for each formulation. Normal saline was used as the receiver solution. The entire receiver solution was collected and replaced with fresh saline at each time point. The receiver solution collected was analyzed by an HPLC for the concentration of ibuprofen. The cumulative amount of ibuprofen across human cadaver skin was calculated using the measured ibuprofen concentrations in the receiver solutions, which were plotted versus time and are described below.

The cumulative amount of ibuprofen across human cadaver skin at 24 hours increased from 0.33 mg/cm² to 5.74 mg/cm² when the calculated excess NaOH concentration in the gel was increased from 0% to 1.74% (Ibu-4), i.e., up to about 17-fold more flux was obtained than with the formulation having 0% excess NaOH concentration. The cumulative amount of ibuprofen that permeated across the human cadaver skin at 24 hours from the formulation with an excess NaOH concentration of 0.98% (Ibu-3, 1.12 mg/cm²) is about 3 times higher than that from the formulation with an excess NaOH concentration of 0% (Ibu-2, 0.33 mg/cm²).

### Example 6

An in vitro skin permeation study was conducted using four phenylpropanolamine hydrochloride transdermal systems, designated PPA-5, PPA-6, PPA-7, and PPA-8, the compositions of which are set forth in Table 15. The matrix patches were prepared and evaluated using the same procedures as set forth in Example 3. The theoretical percent weight for each ingredient after drying (calculated assuming all the volatile ingredients were completely removed during drying) is listed in Table 16.

TABLE 15
Component Weight and Weight Percent Based on Total Solution
Weight

| | PPA-5 | PPA-6 | PPA-7 | PPA-8 |
|---|---|---|---|---|
| | g (wt %) | g (wt %) | g (wt %) | g (wt %) |
| PPA-HCl | 0.5 (6.7) | 0.5 (5.7) | 0.5 (5.6) | 0.5 (5.5) |
| Na₂CO₃ | 0 | 0.29 (3.3) | 0.44 (5.0) | 0.74 (8.1) |
| DI water | 1.0 (13.5) | 2.0 (23.0) | 2.0 (22.6) | 2.0 (21.9) |
| Methyl alcohol | 0.5 (6.7) | 0.5 (5.7) | 0.5 (5.6) | 0.5 (5.5) |
| PG | 0.2 (2.7) | 0.2 (2.3) | 0.2 (2.3) | 0.2 (2.2) |
| HPMC | 0.01 (0.1) | 0.01 (0.1) | 0.01 (0.1) | 0.01 (0.1) |
| Heptane | 1.2 (16.2) | 1.2 (13.8) | 1.2 (13.6) | 1.2 (13.1) |
| PIB adhesive | 4 (54.0) | 4 (46.0) | 4 (45.2) | 4 (45.2) |
| (30% solid) | | | | |

TABLE 16
Weight and Theoretical Weight Percent Based on Dried Film Weight

| | PPA-5 | PPA-6 | PPA-7 | PPA-8 |
|---|---|---|---|---|
| | g (wt %) | g (wt %) | g (wt %) | g (wt %) |
| PPA-HCl | 0.5 (26.2) | 0.5 (22.7) | 0.5 (21.3) | 0.5 (18.9) |
| Na₂CO₃ | 0 | 0.29 (13.2) | 0.44 (18.7) | 0.74 (27.9) |
| PG | 0.2 (10.5) | 0.2 (9.1) | 0.2 (8.5) | 0.2 (7.5) |
| HPMC | 0.01 (0.5) | 0.01 (0.5) | 0.01 (0.4) | 0.01 (0.4) |
| PIB adhesive | 1.2 (62.8) | 1.2 (54.5) | 1.2 (51.1) | 1.2 (45.3) |
| (30% solid) | | | | |

Since PPA-HCl is a salt of a free base, it reacts with Na₂CO₃. The concentration of Na₂CO₃ in the system after the reaction is completed depends on the amount of PPA-HCl added. The remaining sodium carbonate concentration after the reaction is completed is defined as “excess Na₂CO₃ concentration,” which is defined by the following equation.

[Na₂CO_{3 excess}]=[Na₂CO_{3 total}]−[Na₂CO_{3 needed for neutralization}]

The excess Na₂CO₃ for these four PPA-HCl systems was calculated, and the pH was measured as described in the Methods section. The pH of the PPA-HCl patch increased from 9.81 to 10.17 when the calculated excess Na₂CO₃ concentration in the dried patch was increased from 0.4% to 16.7%.

TABLE 17
Excess Na₂CO₃ Concentration (wt %) and pH

| | PPA-5 | PPA-6 | PPA-7 | PPA-8 |
|---|---|---|---|---|
| Excess Na₂CO₃ | — | 0.4% | 6.7% | 16.7% |
| Concentration | | | | |
| pH | 6.54 | 9.81 | 9.86 | 10.17 |

The cumulative amount of PPA-HCl across human cadaver skin was calculated using the measured PPA-HCl concentrations in the receiver solutions.

TABLE 18
Cumulative Amount of PPA-HCl (μg/cm²)

| Time | PPA-5 | PPA-6 | PPA-7 | PPA-8 |
|---|---|---|---|---|
| 5 hours | 152.8 | 68.0 | 81.1 | 144.8 |
| 15 hours | 359.5 | 222.7 | 400.8 | 631.2 |
| 19 hours | 442.7 | 295.7 | 551.5 | 864.3 |
| 24 hours | 545.1 | 410.4 | 705.6 | 1147.5 |

Even though patch PPA-6 contained 13.2% Na₂CO₃, the cumulative amount of PPA-HCl that permeated across the human cadaver skin at 24 hours (410.4 μg/cm²) was lower than that from the formulation without Na₂CO₃ (PPA-5, 545.1 μg/cm²). This may be due to the consumption of Na₂CO₃ by the reaction between Na₂CO₃ and PPA-HCl, which reduced the Na₂CO₃ concentration to only 0.4% as the excess Na₂CO₃ concentration. When the calculated excess Na₂CO₃ concentration in the dried patch was further increased from 0.4% to 16.7%, the cumulative amount of PPA-HCl that permeated across the human cadaver skin at 24 hours was increased from 410.4 to 1147.5 μg/cm². This result indicated that the permeation of PPA-HCl could be enhanced by Na₂CO₃, even though the required excess Na₂CO₃ concentration is higher than that of NaOH. Greater amounts of Na₂CO₃ may be necessary because it is a weaker base compared to NaOH and the molecular weight of Na₂CO₃ is higher than that of NaOH. The formulation of PPA-7 provided up to 1.3-fold more phenylpropanolamine hydrochloride flux than in the absence of Na₂CO₃ (PPA-PC1). The highest pH formulation evaluated, PPA-8, provided up to 2-fold more flux than in the absence of Na₂CO₃.

### Example 7

An in vitro skin permeation study was conducted using four phenylpropanolamine hydrochloride transdermal systems, designated PPA-9, PPA-10, PPA-11, and PPA-12, the compositions of which are set forth in Table 19. The matrix patches were prepared and evaluated using the same procedures as set forth in Example 3. The theoretical percent weight for each ingredient after drying (calculated assuming all the volatile ingredients were completely removed during drying) is listed in Table 20.

TABLE 19
Component Weight and Weight Percent Based on Total Solution
Weight

| | PPA-9 | PPA-10 | PPA-11 | PPA-12 |
|---|---|---|---|---|
| | g (wt %) | g (wt %) | g (wt %) | g (wt %) |
| PPA-HCl | 0.5 (6.6) | 0.5 (6.1) | 0.5 (6.1) | 0.5 (6.1) |
| K₃PO₄ | 0 | 0.57 (7.0) | 0.6 (7.3) | 0.66 (8.0) |
| DI water | 1.0 (13.2) | 1.0 (12.2) | 1.0 (12.2) | 1.0 (12.1) |
| PG | 0.5 (6.6) | 0.5 (6.1) | 0.5 (6.1) | 0.5 (6.1) |
| Methyl alcohol | 0.5 (6.6) | 0.5 (6.1) | 0.5 (6.1) | 0.5 (6.1) |
| PIB adhesive | 4 (52.6) | 4 (49.0) | 4 (48.8) | 4 (48.4) |
| (30% solid) | | | | |
| HPMC | 0.1 (1.3) | 0.1 (1.2) | 0.1 (1.2) | 0.1 (1.2) |
| Heptane | 1 (13.2) | 1 (12.2) | 1 (12.2) | 1 (12.1) |

TABLE 20
Weight and Theoretical Weight Percent Based on Dried Film Weight

| | PPA-9 | PPA-10 | PPA-11 | PPA-12 |
|---|---|---|---|---|
| | g (wt %) | g (wt %) | g (wt %) | g (wt %) |
| PPA-HCl | 0.5 (21.7) | 0.5 (17.4) | 0.5 (17.2) | 0.5 (16.9) |
| K₃PO₄ | 0 | 0.57 (19.9) | 0.6 (20.7) | 0.66 (22.3) |
| PG | 0.5 (21.7) | 0.5 (17.4) | 0.5 (17.2) | 0.5 (16.9) |
| PIB adhesive | 1.2 (52.2) | 1.2 (41.8) | 1.2 (41.4) | 1.2 (40.5) |
| HPMC | 0.1 (4.3) | 0.1 (3.5) | 0.1 (3.4) | 0.1 (3.4) |

Since PPA-HCl is a salt of a free base, it reacts with K₃PO₄. The concentration of K₃PO₄ in the system after the reaction is completed depends on the amount of PPA-HCl added. The remaining K₃PO₄ concentration after the reaction is completed is defined as “excess K₃PO₄ concentration,” which is defined by the following equation.

[K₃PO_{4 excess}]=[K₃PO_{4 total}]−[K₃PO_{4 needed for neutralization}]

The excess K₃PO₄ concentration for the four PPA-HCl systems was calculated, and the pH of the patch was measured as described in the Methods section. The pH of the PPA-HCl patch increased from 6.75 to 9.68 when the K₃PO₄ concentration in the dried patch was increased from 0% to 19.9% (or 0.2% excess K₃PO₄ concentration). However, the pH of the PPA-HCl patch remained about the same when the excess K₃PO₄ concentration in the dried patch was further increased from 0.2% to 3.2%.

TABLE 21
Excess K₃PO₄ Concentration (wt %) and pH

| | PPA-9 | PPA-10 | PPA-11 | PPA-12 |
|---|---|---|---|---|
| Excess K₃PO₄ | — | 0.2% | 1.2% | 3.2% |
| Concentration | | | | |
| pH | 6.75 | 9.68 | 9.62 | 10.08 |

The cumulative amount of PPA-HCl across human cadaver skin was calculated using the measured PPA-HCl concentrations in the receiver solutions.

TABLE 22
Cumulative Amount of PPA-HCl (μg/cm²)

| Time | PPA-9 | PPA-10 | PPA-11 | PPA-12 |
|---|---|---|---|---|
| 5 hours | 94.7 | 660.0 | 421.6 | 362.9 |
| 16 hours | 445.9 | 1701.3 | 1420.3 | 1607.5 |
| 20 hours | 576.8 | 1919.2 | 1633.1 | 1872.5 |
| 24 hours | 680.5 | 2055.2 | 1762.9 | 2021.1 |

The cumulative amount of PPA-HCl that permeated across the human cadaver skin at 24 hours for PPA-10 (2055.2 μg/cm²) with a calculated excess K₃PO₄ concentration of 0.2% was three times higher than that from the formulation without K₃PO₄ (PPA-9, 680.5 μg/cm²). This result indicated that the permeation of PPA-HCl could be enhanced with an excess K₃PO₄ concentration as low as 0.2%.

The cumulative amount of PPA-HCl across human cadaver skin at 24 hours remained about the same when the excess K₃PO₄ concentration in the dried patch was increased from 0.2% to 3.2%. The formulations of PPA-10, PPA-11 and PPA-12, all provided up to 3-fold more phenylpropanolamine hydrochloride flux than in the absence of K₃PO₄ (PPA-9).

### Example 8

An in vitro skin permeation study was conducted using four phenylpropanolamine hydrochloride transdermal systems, designated PPA-13, PPA-14, PPA-15, and PPA-16, the compositions of which are set forth in Table 23. The matrix patches were prepared and evaluated using the same procedures as set forth in Example 3. The theoretical percent weight for each ingredient after drying (calculated assuming all the volatile ingredients were completely removed during drying) is listed in Table 24.

TABLE 23
Component Weight and Weight Percent Based on Total Solution
Weight

| | PPA-13 | PPA-14 | PPA-15 | PPA-16 |
|---|---|---|---|---|
| | g (wt %) | g (wt %) | g (wt %) | g (wt %) |
| PPA-HCl | 0.5 (6.9) | 0.5 (6.4) | 0.5 (6.3) | 0.5 (6.1) |
| K₃PO₄ | 0 | 0.57 (7.3) | 0.73 (9.2) | 1.05 (12.7) |
| DI water | 1.0 (13.9) | 1.0 (12.9) | 1.0 (12.6) | 1.0 (12.1) |
| Methyl alcohol | 0.5 (6.9) | 0.5 (6.4) | 0.5 (6.3) | 0.5 (6.1) |
| PG | 0.2 (2.8) | 0.2 (2.6) | 0.2 (2.5) | 0.2 (2.4) |
| HPMC | 0.01 (0.1) | 0.01 (0.1) | 0.01 (0.1) | 0.01 (0.1) |
| Heptane | 1 (13.9) | 1 (12.9) | 1 (12.6) | 1 (12.1) |
| PIB adhesive | 4 (55.5) | 4 (51.4) | 4 (50.4) | 4 (48.4) |
| (30% solid) | | | | |

TABLE 24
Weight and Theoretical Weight Percent Based on Dried Film Weight

| | PPA-13 | PPA-14 | PPA-15 | PPA-16 |
|---|---|---|---|---|
| | g (wt %) | g (wt %) | g (wt %) | g (wt %) |
| PPA-HCl | 0.5 (26.2) | 0.5 (20.2) | 0.5 (18.9) | 0.5 (16.5) |
| K₃PO₄ | 0 | 0.57 (23.6) | 0.73 (27.7) | 1.05 (35.5) |
| PG | 0.2 (10.5) | 0.2 (8.1) | 0.2 (7.6) | 0.2 (6.8) |
| HPMC | 0.01 (0.5) | 0.01 (0.4) | 0.01 (0.4) | 0.01 (0.3) |
| PIB adhesive | 1.2 (62.8) | 1.2 (48.4) | 1.2 (45.5) | 1.2 (40.5) |

The excess K₃PO₄ concentration for four PPA-HCl systems was calculated as described in Example 7, and the pH was measured as described in the Methods section. The pH of the PPA-HCl patch increased from 7 to 9.72 when the K₃PO₄ concentration in the dried patch was increased from 0% to 23% (or 0.2% excess K₃PO₄ concentration). The pH of the PPA-HCl patch increased from 9.72 to 10.44 when the excess K₃PO₄ concentration in the dried patch was further increased from 0.2% to 16.4%.

TABLE 25
Excess K₃PO₄ Concentration (wt %) and pH

| | PPA-13 | PPA-14 | PPA-15 | PPA-16 |
|---|---|---|---|---|
| Excess K₃PO₄ | — | 0.2% | 6.2% | 16.4% |
| Concentration | | | | |
| pH | 7 | 9.72 | 10.17 | 10.44 |

The cumulative amount of PPA-HCl across human cadaver skin was calculated using the measured PPA-HCl concentrations in the receiver solutions.

TABLE 26
Cumulative Amount of PPA-HCl (μg/cm²)

| Time | PPA-13 | PPA-14 | PPA-15 | PPA-16 |
|---|---|---|---|---|
| 5 hours | 336.8 | 553.1 | 291.5 | 186.7 |
| 16 hours | 879.5 | 1702.4 | 1172.5 | 873.1 |
| 20 hours | 1091.2 | 2031.2 | 1711.5 | 1204.3 |
| 24 hours | 1324.0 | 2378.4 | 2222.7 | 1628.0 |

The cumulative amount of PPA-HCl that permeated across the human cadaver skin at 24 hours for PPA-14 (2378.4 μg/cm²) with a calculated excess K₃PO₄ concentration of 0.2% was about two times higher than that from the formulation without K₃PO₄ (PPA-13, 1324.0 μg/cm²). This result indicated that the permeation of PPA-HCl is enhanced with an excess K₃PO₄ concentration as low as 0.2%.

The cumulative amount of PPA-HCl across human cadaver skin at 24 hours remained about the same when the excess K₃PO₄ concentration in the dried patch was increased from 0.2% to 6.2%. When the excess K₃PO₄ concentration in the dried patch was further increased from 6.2% to 16.4%, the cumulative amount of PPA-HCl across human cadaver skin at 24 hours decreased from 2222.7 to 1628.0 μg/cm². This decrease in flux may be because the high concentration of K₃PO₄ made the adhesive matrix more hydrophobic and the amount of K₃PO₄ that could be dissolved by the small amount of water on the top of the skin was reduced.

The formulation of PPA-14 provided up to 2-fold more phenylpropanolamine hydrochloride flux than in the absence of K₃PO₄ (PPA-13), while PPA-15 provided up to 1.5-fold increase in flux.

### Example 9

An in vitro skin permeation study was conducted using four estradiol transdermal systems, designated Est-4, Est-5, Est-6, and Est-7, the compositions of which are set forth in Table 27. The matrix patches were prepared and evaluated using the same procedures as set forth in Example 1. The theoretical percent weight for each ingredient after drying (calculated assuming all the volatile ingredients were completely removed during drying) is listed in Table 28.

TABLE 27
Component Weight and Weight Percent Based on Total Solution
Weight

| | Est-4 | Est-5 | Est-6 | Est-7 |
|---|---|---|---|---|
| | g (wt %) | g (wt %) | g (wt %) | g (wt %) |
| Estradiol | 0.03 (0.5) | 0.03 (0.5) | 0.03 (0.5) | 0.03 (0.4) |
| Methyl alcohol | 0.5 (8.0) | 0.5 (7.8) | 0.5 (7.6) | 0.5 (7.4) |
| K₃PO₄ | 0 | 0.1 (1.6) | 0.3 (4.6) | 0.48 (7.1) |
| DI water | 0.5 (8.0) | 0.5 (7.8) | 0.5 (7.6) | 0.5 (7.4) |
| PG | 0.25 (4.0) | 0.25 (3.9) | 0.25 (3.8) | 0.25 (3.7) |
| PIB adhesive | 4 (63.7) | 4 (62.7) | 4 (60.8) | 4 (59.2) |
| (30% solid) | | | | |
| Heptane | 1 (15.9) | 1 (15.7) | 1 (15.2) | 1 (14.8) |

TABLE 28
Component Weight and Weight Percent Based on Dried Film Weight

| | Est-4 | Est-5 | Est-6 | Est-7 |
|---|---|---|---|---|
| | g (wt %) | g (wt %) | g (wt %) | g (wt %) |
| Estradiol | 0.03 (2.0) | 0.03 (1.9) | 0.03 (1.7) | 0.03 (1.5) |
| K₃PO₄ | 0 | 0.1 (6.3) | 0.3 (16.9) | 0.48 (24.5) |
| PG | 0.25 (16.9) | 0.25 (15.8) | 0.25 (14.0) | 0.25 (12.8) |
| PIB adhesive | 1.2 (81.1) | 1.2 (76.0) | 1.2 (67.4) | 1.2 (61.2) |

Since estradiol is not expected to react with K₃PO₄, the K₃PO₄ concentration listed in Table 28 equals the excess K₃PO₄ concentration, calculated as described in Example 7.

The pH of each patch was measured as described in the Methods section. The pH of the estradiol patch increased from 6.4 to 10.83 when the K₃PO₄ concentration in the dried patch was increased from 0% to 16.9%. However, the pH of the estradiol patch decreased from 10.83 to 9.87 when the K₃PO₄ concentration in the dried patch was further increased from 16.9% to 24.5%.

TABLE 29
Excess K₃PO₄ Concentration (wt %) and pH

| | Est-4 | Est-5 | Est-6 | Est-7 |
|---|---|---|---|---|
| Excess K₃PO₄ | 0% | 6.3% | 16.9% | 24.5% |
| Concentration | | | | |
| pH | 6.4 | 8.89 | 10.83 | 9.87 |

The cumulative amount of estradiol across human cadaver skin was calculated using the measured estradiol concentrations in the receiver solutions.

TABLE 30
Cumulative Amount of Estradiol (μg/cm²)

| Time | Est-4 | Est-5 | Est-6 | Est-7 |
|---|---|---|---|---|
| 5 hours | 0.2 | 1.2 | 2.1 | 1.5 |
| 16.5 hours | 0.4 | 3.9 | 7.6 | 3.7 |
| 20 hours | 0.5 | 4.6 | 8.8 | 4.4 |
| 24 hours | 0.6 | 5.6 | 10.2 | 5.3 |

The cumulative amount of estradiol that permeated across the human cadaver skin at 24 hours for Est-5 (5.6 μg/cm²) with a calculated excess K₃PO₄ concentration of 6.3% was about nine times higher than that from the formulation without K₃PO₄ (Est-PK1, 0.6 μg/cm²). This result indicated that the permeation of estradiol is enhanced by K₃PO₄. The cumulative amount of estradiol across human cadaver skin at 24 hours increased from 5.6 to 10.2 when the excess K₃PO₄ concentration in the dried patch was increased from 6.3% to 16.9%. When the excess K₃PO₄ concentration in the dried patch was further increased from 16.9% to 24.5%, the cumulative amount of estradiol across human cadaver skin at 24 hours decreased from 10.2 to 5.3 μg/cm². This decrease in flux may be because the high concentration of K₃PO₄ made the adhesive matrix more hydrophobic and the amount of K₃PO₄ that could be dissolved by the small amount of water on the top of the skin was reduced.

The formulations of Est-5 and Est-7 provided up to 9-fold more estradiol flux than in the absence of K₃PO₄ (Est-4). The formulation of Est-6 provided up to 17-fold more flux than in the absence of K₃PO₄.

### Example 10

An in vitro skin permeation study was conducted using four estradiol transdermal systems, designated Est-11, Est-12, Est-13, and Est-14, the compositions of which are set forth in Table 31. The matrix patches were prepared and evaluated using the same procedures as set forth in Example 1. The theoretical percent weight for each ingredient after drying (calculated assuming all the volatile ingredients were completely removed during drying) is listed in Table 32.

TABLE 31
Component Weight and Weight Percent Based on Total Solution
Weight

| | Est-11 | Est-12 | Est-13 | Est-14 |
|---|---|---|---|---|
| | g (wt %) | g (wt %) | g (wt %) | g (wt %) |
| Estradiol | 0.03 (0.5) | 0.03 (0.4) | 0.03 (0.4) | 0.03 (0.4) |
| Na₂CO₃ | 0 | 0.11 (1.6) | 0.3 (4.1) | 0.45 (6.1) |
| DI water | 0.5 (8.0) | 1.2 (16.9) | 1.2 (16.5) | 1.2 (16.2) |
| Methyl alcohol | 0.5 (8.0) | 0.5 (7.1) | 0.5 (6.9) | 0.5 (6.7) |
| PIB adhesive | 4 (63.7) | 4 (56.4) | 4 (55.0) | 4 (53.8) |
| (30% solid) | | | | |
| PG | 0.25 (4.0) | 0.25 (3.5) | 0.25 (3.4) | 0.25 (3.4) |
| Heptane | 1 (15.9) | 1 (14.1) | 1 (13.7) | 1 (13.5) |

TABLE 32
Component Weight and Weight Percent Based on Total Solution
Weight

| | Est-11 | Est-12 | Est-13 | Est-14 |
|---|---|---|---|---|
| | g (wt %) | g (wt %) | g (wt %) | g (wt %) |
| Estradiol | 0.03 (2.0) | 0.03 (1.9) | 0.03 (1.7) | 0.03 (1.6) |
| Na₂CO₃ | 0 | 0.11 (6.9%) | 0.3 (16.9%) | 0.45 (23.3) |
| PIB adhesive | 1.2 (81.1) | 1.2 (75.5) | 1.2 (67.4) | 1.2 (62.2) |
| PG | 0.25 (16.9) | 0.25 (15.7) | 0.25 (14.0) | 0.25 (13.0) |

Since estradiol is not expected to react with Na₂CO₃, the Na₂CO₃ concentration listed in Table 32 equals the excess Na₂CO₃ concentration, calculated as described in Example 6.

The pH of each patch was measured as described in the Methods section. The pH of the estradiol patch measured using the procedures listed above increased from 7.48 to 10.51 when the Na₂CO₃ concentration in the dried patch was increased from 0% to 16.9%. However, when the Na₂CO₃ concentration in the dried patch was further increased from 16.9% to 23.3%, the pH of the estradiol patch remained about the same.

TABLE 33
Excess Na₂CO₃ Concentration (wt %) and pH

| | Est-11 | Est-12 | Est-13 | Est-14 |
|---|---|---|---|---|
| Excess Na₂CO₃ | 0% | 6.9% | 16.9% | 23.3% |
| Concentration | | | | |
| pH | 7.48 | 9.87 | 10.51 | 10.49 |

The cumulative amount of estradiol across human cadaver skin was calculated using the measured estradiol concentrations in the receiver solutions.

TABLE 34
Cumulative Amount of Estradiol (μg/cm²)

| Time | Est-11 | Est-12 | Est-13 | Est-14 |
|---|---|---|---|---|
| 5 hours | 0.1 | 0.4 | 0.1 | 0.1 |
| 16.5 hours | 0.2 | 0.9 | 0.4 | 0.6 |
| 20 hours | 0.3 | 1.1 | 0.6 | 1.0 |
| 24 hours | 0.3 | 1.4 | 1.0 | 1.4 |

The cumulative amount of estradiol that permeated across the human cadaver skin at 24 hours for Est-12 (1.4 μg/cm²) with a calculated excess Na₂CO₃ concentration of 6.9% was about four times higher than that from the formulation without Na₂CO₃ (Est-11, 0.3 μg/cm²). This result indicated that Na₂CO₃ could enhance the permeation of estradiol.

The cumulative amount of estradiol across human cadaver skin at 24 hours remained about the same when the excess Na₂CO₃ concentration in the dried patch was increased from 6.9% to 23.3%. This behavior may be because the amount of Na₂CO₃ that could be dissolved by the small amount of water on the top of the skin remained about the same for Est-12, Est-13 and Est-14.

The formulations of Est-12 and Est-14 provided up to 5-fold more estradiol flux than in the absence of Na₂CO₃ (Est-11). The formulation of Est-13 provided up to 3-fold more flux than in the absence of Na₂CO₃.

### Example 11

An in vitro skin permeation study was conducted using four estradiol transdermal systems, designated Est-15, Est-16, Est-17, and Est-18, the compositions of which are set forth in Table 35. The matrix patches were prepared and evaluated using the same procedures as set forth in Example 1. The theoretical percent weight for each ingredient after drying (calculated assuming all the volatile ingredients were completely removed during drying) is listed in Table 36.

TABLE 35
Component Weight and Weight Percent Based on Total Solution
Weight

| | Est-15 | Est-16 | Est-17 | Est-18 |
|---|---|---|---|---|
| | g (wt %) | g (wt %) | g (wt %) | g (wt %) |
| Estradiol | 0.03 (0.5) | 0.03 (0.4) | 0.03 (0.4) | 0.03 (0.4) |
| MgO | 0 | 0.11 (1.6) | 0.3 (4.1) | 0.45 (6.1) |
| DI water | 0.5 (8.0) | 1.2 (16.9) | 1.2 (16.5) | 1.2 (16.2) |
| Methyl alcohol | 0.5 (8.0) | 0.5 (7.1) | 0.5 (6.9) | 0.5 (6.7) |
| PIB adhesive | 4 (63.7) | 4 (56.4) | 4 (55.0) | 4 (53.8) |
| (30% solid) | | | | |
| PG | 0.250 (4.0) | 0.25 (3.5) | 0.25 (3.4) | 0.25 (3.4) |
| Heptane | 1 (15.9) | 1 (14.1) | 1 (13.7) | 1 (13.5) |

TABLE 36
Component Weight and Weight Percent Based on Total Solution
Weight

| | Est-15 | Est-16 | Est-17 | Est-18 |
|---|---|---|---|---|
| | g (wt %) | g (wt %) | g (wt %) | g (wt %) |
| Estradiol | 0.03 (2.0) | 0.03 (1.9) | 0.03 (1.7) | 0.03 (1.6) |
| MgO | 0 | 0.11 (6.9) | 0.3 (16.9) | 0.45 (23.3) |
| PIB adhesive | 1.2 (81.1) | 1.2 (75.5) | 1.2 (67.4) | 1.2 (62.2) |
| PG | 0.25 (16.9) | 0.25 (15.7) | 0.25 (14.0) | 0.25 (13.0) |

Since estradiol is not expected to react with MgO, the MgO concentration listed in Table 36 equals the excess MgO concentration, calculated as described in Example 12.

The pH of each patch was measured as described in the Methods section. The pH of the estradiol patch measured using the procedures listed above increased from 7.48 to 10.28 when the MgO concentration in the dried patch was increased from 0% to 23.3%.

TABLE 37
Excess MgO Concentration (wt %) and pH

| | Est-15 | Est-16 | Est-17 | Est-18 |
|---|---|---|---|---|
| Excess MgO | 0% | 6.9% | 16.9% | 23.3% |
| Concentration | | | | |
| pH | 7.48 | 8.95 | 9.66 | 10.28 |

The cumulative amount of estradiol across human cadaver skin was calculated using the measured estradiol concentrations in the receiver solutions.

TABLE 38
Cumulative Amount of Estradiol (μg/cm²)

| Time | Est-15 | Est-16 | Est-17 | Est-18 |
|---|---|---|---|---|
| 4.75 hours | 0.08 | 0.09 | 0.05 | 0.02 |
| 15.75 hours | 0.21 | 0.31 | 0.19 | 0.13 |
| 19.75 hours | 0.26 | 0.41 | 0.26 | 0.19 |
| 23.75 hours | 0.32 | 0.53 | 0.36 | 0.27 |

The cumulative amount of estradiol that permeated across the human cadaver skin at 24 hours for Est-16 (0.53 μg/cm²) with a calculated excess MgO concentration of 6.9% was slightly higher than that from the formulation without MgO (Est-15, 0.32 μg/cm²). Thus, the formulation of Est-16 provided up to 2-fold more estradiol flux than in the absence of MgO (Est-15). This result indicated that MgO enhances the permeation of estradiol.

The cumulative amount of estradiol across human cadaver skin at 24 hours decreased from 0.53 to 0.27 μg/cm when the excess MgO concentration in the dried patch was increased from 6.9% to 23.3%. This behavior may be because the high concentration of MgO made the adhesive matrix more hydrophobic and the amount of MgO that could be dissolved by the small amount of water on the top of the skin was reduced.

### Example 12

An in vitro skin permeation study was conducted using four phenylpropanolamine hydrochloride transdermal systems, designated PPA-17, PPA-18, PPA-19, and PPA-20, the compositions of which are set forth in Table 39. The matrix patches were prepared and evaluated using the same procedures as set forth in Example 3. The theoretical percent weight for each ingredient after drying (calculated assuming all the volatile ingredients were completely removed during drying) is listed in Table 40.

TABLE 39
Component Weight and Weight Percent Based on Total Solution Weight

| | PPA-17 | PPA-18 | PPA-19 | PPA-20 |
|---|---|---|---|---|
| | g (wt %) | g (wt %) | g (wt %) | g (wt %) |
| PPA-HCl | 0.5 (6.9) | 0.5 (6.0) | 0.5 (5.9) | 0.5 (5.7) |
| MgO | 0 | 0.11 (1.3) | 0.26 (3.1) | 0.50 (5.7) |
| DI water | 1.0 (13.9) | 2.0 (24.0) | 2.0 (23.6) | 2.0 (22.9) |
| Methyl alcohol | 0.5 (6.9) | 0.5 (6.0) | 0.5 (5.9) | 0.5 (5.7) |
| PG | 0.2 (2.8) | 0.2 (2.4) | 0.2 (2.4) | 0.2 (2.3) |
| HPMC | 0.02 (0.3) | 0.02 (0.2) | 0.02 (0.2) | 0.02 (0.2) |
| PIB adhesive | 4 (55.4) | 4 (48.0) | 4 (47.2) | 4 (45.9) |
| (30% solid) | | | | |
| Heptane | 1.0 (13.9) | 1.0 (12.0) | 1.0 (11.8) | 1.0 (11.5) |

TABLE 40
Component Weight and Weight Percent Based on Dried Film Weight

| | PPA-17 | PPA-18 | PPA-19 | PPA-20 |
|---|---|---|---|---|
| | g (wt %) | g (wt %) | g (wt %) | g (wt %) |
| PPA-HCl | 0.5 (26.0) | 0.5 (24.6) | 0.5 (22.9) | 0.5 (20.7) |
| MgO | 0 | 0.11 (5.4) | 0.26 (11.9) | 0.50 (20.7) |
| PG | 0.2 (10.4) | 0.2 (9.9) | 0.2 (9.2) | 0.2 (8.3) |
| HPMC | 0.02 (1.0) | 0.02 (1.0) | 0.02 (0.9) | 0.02 (0.8) |
| PIB adhesive | 1.2 (62.5) | 1.2 (59.1) | 1.2 (55.0) | 1.2 (49.6) |

The cumulative amount of PPA-HCl across human cadaver skin was calculated using the measured PPA-HCl concentrations in the receiver solutions.

TABLE 41
Cumulative Amount of PPA-HCl (μg/cm²)

| Time | PPA-17 | PPA-18 | PPA-19 | PPA-20 |
|---|---|---|---|---|
| 5 hours | 18.7 | 296.8 | 222.1 | 489.4 |
| 15 hours | 77.8 | 621.5 | 1362.9 | 1255.2 |
| 19 hours | 102.7 | 711.4 | 1920.9 | 1524.9 |
| 24 hours | 129.8 | 801.9 | 2533.4 | 1831.3 |

Since PPA-HCl is a salt of a free base, it reacts with MgO. The concentration of MgO in the system after the reaction is completed depends on the amount of PPA-HCl added. The remaining MgO concentration after the reaction is completed is defined as “excess MgO concentration,” which is defined by the following equation.

[MgO_{excess}]=[MgOₜₒₜₐₗ]−[MgO_{needed for neutralization}]

The excess MgO concentration for four PPA-HCl systems was calculated, and the pH of the patch was measured as described in the Methods section. The pH of the PPA-HCl patch increased from 7.89 to 9.60 when the MgO concentration in the dried patch was increased from 0% to 5.4% (or 0.1% excess MgO concentration). The pH of the PPA-HCl remained about the same when the excess MgO concentration in the dried patch was further increased from 0.1% to 16.2%.

TABLE 42
Excess MgO Concentration (wt %) and pH

| | PPA-17 | PPA-18 | PPA-19 | PPA-20 |
|---|---|---|---|---|
| Excess MgO | — | 0.1% | 7.0% | 16.2% |
| Concentration | | | | |
| pH | 7.89 | 9.60 | 10.09 | 10.10 |

The cumulative amount of PPA-HCl that permeated across the human cadaver skin at 24 hours for PPA-18 (801.9 μg/cm²) with a calculated excess MgO concentration of 0.1% was about six times higher than that from the formulation without MgO (PPA-17, 129.8 μg/cm²). This result indicated that the permeation of PPA-HCl is enhanced with an excess MgO concentration as low as 0.1%.

The cumulative amount of PPA-HCl across human cadaver skin at 24 hours increased from 801.9 to 2533.4 μg/cm² when the excess MgO concentration in the dried patch was increased from 0.1% to 7.0%. When the excess MgO concentration in the dried patch was further increased from 7.0% to 16.2%, the cumulative amount of PPA-HCl across human cadaver skin at 24 hours decreased from 2533.4 to 1831.3 μg/cm². This decrease in flux may be because the high concentration of MgO made the adhesive matrix more hydrophobic and the amount of MgO that could be dissolved by the small amount of water on the top of the skin was reduced.

The formulation of PPA-18 provided up to 6-fold more phenylpropanolamine hydrochloride flux than in the absence of MgO (PPA-17). The formulation of PPA-19 provided up to 20-fold more flux, while PPA-20 provided up to 14-fold more flux than in the absence of MgO.

### Example 13

An in vitro skin permeation study was conducted using three leuprolide solutions, designated Leu-1, Leu-2, and Leu-3, the compositions of which are set forth in Table 43. Each formulation was stirred until the solution was uniform.

TABLE 43
Component Weight and Weight Percent Based on Total Solution Weight

| | Leu-1 | Leu-2* | Leu-3* |
|---|---|---|---|
| | g (wt %) | g (wt %) | |
| Leuprolide | 0.003 (0.4) | 6.4 × 10⁻⁴ (0.18) | 6.4 × 10⁻⁴ (0.16) |
| DI water | 0.45 (64.0) | 0.28 (80.9) | 0.33 (80.3) |
| NaOH | 0 | 0.0125 (3.6) | 0.0275 (6.7) |
| PG | 0.25 (35.6) | 0.053 (15.3) | 0.053 (13.0) |

*Solutions Leu-2 and Leu-3 were prepared using 0.15 g of Leu-1, then adding the correct amount of NaOH and DI water. Percentages may not add up to 100% due to rounding.

The in-vitro permeation of each leuprolide solution through human cadaver skin was performed using Franz-type diffusion cells with a diffusion area of 1 cm². The volume of receiver solution was 8 ml. Human cadaver skin was cut to a proper size and placed on a flat surface with the stratum corneum side facing up. The skin was clamped between the donor and receiver chambers of the diffusion cell, and the stratum corneum was allowed to dry. The leuprolide solution was applied to the stratum corneum using a micro-pipette. Each formulation was applied in a 25 μl dosage and a 50 μl dosage for a total of 6 test groups. The receiver chamber was sealed to the atmosphere using parafilm wrap so that it was spill-proof and airtight. Three diffusion cells were used for each test group for a total of 18 cells.

The cells were filled with DI water for a receiver solution. The DI water had been degased to remove air bubbles. The receiver solution was completely withdrawn and replaced with fresh DI water at each time point. Samples of the receiver solution were taken and analyzed by HPLC (high pressure liquid chromatography) to determine the leuprolide concentration. The cumulative amount of leuprolide across human cadaver skin was calculated from a 25 μl and a 50 μl solution containing NaOH, using the measured leuprolide concentrations in the receiver solutions for each time point (5 and 24 hours)

TABLE 44
Cumulative Amount of Leuprolide (μg/cm²)

| | 25 μl solution | | | 50 μl solution | | |
|---|---|---|---|---|---|---|
| Time | Leu-1 | Leu-2 | Leu-3 | Leu-1 | Leu-2 | Leu-3 |
| 5 hours | 0.38 | 0.52 | 0.58 | 0.32 | 0.62 | 0.3 |
| 24 hours | 0.52 | 3.21 | 4.43 | 0.32 | 8.58 | 10.8 |

The cumulative amount of leuprolide across human cadaver skin for the 25 μl dosage at 24 hours increased from 0.52 μg/cm² to 4.43 μg/cm² when the calculated sodium hydroxide concentration in the dried patch was increased from 0% to 6.7%. The cumulative amount of leuprolide across human cadaver skin for the 50 μl dosage at 24 hours increased from 0.32 μg/cm² to 10.8 μg/cm² when the calculated sodium hydroxide concentration in the leuprolide solution was increased from 0% to 6.7%. The cumulative amount of leuprolide across human cadaver skin at 24 hours from the 50 μl dosage group containing 3.6% NaOH (Leu-2) was 8.58 μg/cm², which was about 27 times higher than that from the formulation without NaOH (0.32 μg/cm², Leu-1).

The formulation of Leu-2 provided up to 6-fold (25 μl solution) and to 27-fold (50 μl solution) more leuprolide flux than in the absence of NaOH (Leu-1). The formulation of Leu-3 provided up to 9-fold (25 μl solution) and up to 34-fold (50 μl solution) more flux than in the absence of NaOH.

### Example 14

The in-vitro permeation of oxytocin through human cadaver skin was performed using Franz-type diffusion cells with a diffusion area of 1 cm². The volume of receiver solution was 8 ml. Human cadaver skin was cut to a proper size and placed on a flat surface with the stratum corneum side facing up. The skin was clamped between the donor and receiver chambers of the diffusion cell. Eighteen diffusion cells were used in this study. A 2% NaOH aqueous solution (50 μl) was introduced to the donor chambers of nine cells (cells #1 to 9) and a 4% NaOH aqueous solution (50 μl) was introduced to the donor chambers of the other nine cells (cells #10 to 18). Once the NaOH solution is applied, the donor chamber was covered with parafilm.

After 5 hours, the NaOH solution was washed away from the skin for 3 cells (cells #1 to 3) that were treated with 2% NaOH solution and 3 cells (cells #10 to 12) that were treated with 4% NaOH solution. After 10 hours, the NaOH solution was washed away from the skin for 3 cells (cells #4 to 6) that were treated with 2% NaOH solution and 3 cells (cells #13 to 15) that were treated with 4% NaOH solution. After 24 hours, the NaOH solution was washed away from the skin for 3 cells (cells #7 to 9) that were treated with 2% NaOH solution and 3 cells (cells #16 to 18) that were treated with 4% NaOH solution. To wash away the NaOH solution, the receiving fluid was removed and replaced with fresh DI water. This was done twice. DI water was added to the donor chamber to dilute the NaOH solution and then the donor solution was removed. This was repeated several times.

After the NaOH solution was washed away from the skin, the solution in the donor chamber was completely removed and replaced by 50 μl of an oxytocin solution. The formulation of the oxytocin solution is listed in Table 45. Once the oxytocin solution was applied, the donor chamber was covered with parafilm.

TABLE 45
Formulation for the Oxytocin Solution

| Ingredient | g |
|---|---|
| Oxytocin | 0.005 |
| DI water | 0.6 |
| PG | 0.6 |

The cells were filled with DI water as a receiver solution. The DI water had been degased to remove air bubbles. The receiver solution was completely withdrawn and replaced with fresh DI water at each time point. The samples taken were analyzed by HPLC for the concentration of oxytocin in the receiver solution. The cumulative amount of oxytocin across human cadaver skin was calculated using the measured oxytocin concentrations in the receiver solutions for each time point, which were listed in Table 46. The skin was pretreated with 4% NaOH for the specified pretreatment time period.

TABLE 46
Cumulative Amount of Oxytocin (μg/cm²)

| Time | 5 hr Pretreatment | 15 hr Pretreatment | 24 hr Pretreatment |
|---|---|---|---|
| 5 hours | 118.95 | 202.28 | 193.82 |
| 15 hours | 200.66 | 222.45 | 232.72 |
| 24 hours | 225.52 | 231.58 | 236.80 |

### Example 15

The in-vitro permeation of oxytocin through human cadaver skin was performed as described in Example 14, except that a 0.25% NaOH aqueous solution (50 μl) was introduced to the donor chambers of nine cells (cells #1 to 9) and a 1.0% NaOH aqueous solution (50 μl) was introduced to the donor chambers of the other nine cells (cells #10 to 18).

After 5 hours, the NaOH solution was washed away from the skin for 3 cells (cells #1 to 3) that were treated with 0.5% NaOH solution and 3 cells (cells #10 to 12) that were treated with 1.0% NaOH solution. After 11 hours, the NaOH solution was washed away from the skin for 3 cells (cells #4 to 6) that were treated with 0.25% NaOH solution and 3 cells (cells #13 to 15) that were treated with 1.0% NaOH solution. After 24 hours, the NaOH solution was washed away from the skin for 3 cells (cells #7 to 9) that were treated with 0.25% NaOH solution and 3 cells (cells #16 to 18) that were treated with 1.0% NaOH solution. To wash away the NaOH solution, the receiving fluid was removed and replaced with fresh DI water. This was done twice. DI water was added to the donor chamber to dilute the NaOH solution and then the donor solution was removed. This was repeated several times until the pH of donor solution was less than 8.

After the NaOH solution was washed away from the skin, the solution in the donor chamber was completely removed and replaced by 50 μl of an oxytocin solution. The formulation of the oxytocin solution is listed in Table 47. Once the oxytocin solution is applied, the donor chamber was covered with parafilm.

TABLE 47
Formulation for the Oxytocin Solution

| Ingredient | g |
|---|---|
| Oxytocin | 0.005 |
| DI water | 0.6 |
| PG | 0.6 |

The cells were filled with DI water as a receiver solution. The DI water had been degased to remove air bubbles. The receiver solution was completely withdrawn and replaced with fresh DI water at each time point. The samples taken were analyzed by an HPLC for the concentration of oxytocin in the receiver solution. The cumulative amount of oxytocin across human cadaver skin was calculated using the measured oxytocin concentrations in the receiver solutions for each time point, which were listed in Table 48. The skin was pretreated with 1% NaOH for the specified pretreatment time period.

TABLE 48
Cumulative Amount of Oxytocin (μg/cm²)

| | | 11 hr Pretreat- | |
|---|---|---|---|
| Time | 5 hr Pretreatment | ment | 24 hr Pretreatment |
| 4.25 hours | 0.45 | 53.42 | 13.23 |
| 14.75 hours | 0.97 | 67.97 | 21.06 |
| 24 hours | 0.97 | 75.36 | 30.97 |

### Example 16

An in-vitro skin permeation study was conducted using four diclofenac sodium transdermal systems, designated Diclo-1, Diclo-2, Diclo-3, and Diclo-4, the compositions of which are set forth in Table 49. Round disc samples were prepared as described in the Methods section. The theoretical percent weight for each ingredient after drying (calculated assuming all the volatile ingredients were completely removed during drying) is listed in Table 50.

TABLE 49
Component Weight and Weight Percent Based on Total Solution
Weight

| | Diclo-1 | Diclo-2 | Diclo-3 | Diclo-4 |
|---|---|---|---|---|
| | g (wt %) | g (wt %) | g (wt %) | g (wt %) |
| Diclofenac sodium | 0.6 (9.2) | 0.6 (9.1) | 0.6 (9.0) | 0.6 (9.0) |
| PG | 0.9 (13.9) | 0.9 (13.7) | 0.9 (13.6) | 0.9 (13.4) |
| NaOH | 0 | 0.035 (0.5) | 0.05 (0.8) | 0.1 (1.5) |
| PIB adhesive | 4 (61.5) | 4 (60.9) | 4 (60.6) | 4 (59.7) |
| (30% solid) | | | | |
| Heptane | 1 (15.4) | 1 (15.2) | 1 (15.2) | 1 (14.9) |
| DI water | 0 | 0.035 (0.5) | 0.05 (0.8) | 0.1 (1.5) |

TABLE 50
Component Weight and Weight Percent Based on Dried Film Weight

| | Diclo-1 | Diclo-2 | Diclo-3 | Diclo-4 |
|---|---|---|---|---|
| | g (wt %) | g (wt %) | g (wt %) | g (wt %) |
| Diclofenac sodium | 0.6 (22.2) | 0.6 (21.9) | 0.6 (21.8) | 0.6 (21.4) |
| PG | 0.9 (33.3) | 0.9 (32.9) | 0.9 (32.7) | 0.9 (32.1) |
| NaOH | 0 | 0.035 (1.3) | 0.05 (1.8) | 0.1 (3.6) |
| PIB adhesive | 1.2 (44.4) | 1.2 (43.9) | 1.2 (43.6) | 1.2 (42.9) |
| (30% solid) | | | | |

Since diclofenac sodium is not expected to react with NaOH, the NaOH concentration listed in Table 50 equals the excess NaOH concentration, calculated as described in Example 2.

The pH of the patches was measured as described in the Methods section. The pH of the diclofenac sodium patch increased from 7.17 to 11.28 when the calculated excess NaOH concentration in the dried patch was increased from 0% to 3.6%.

TABLE 51
Excess NaOH Concentration (wt %) and pH

| | Diclo-1 | Diclo-2 | Diclo-3 | Diclo-4 |
|---|---|---|---|---|
| Excess NaOH | 0 | 1.3 | 1.8 | 3.6 |
| Concentration | | | | |
| pH | 7.17 | 10.59 | 10.72 | 11.28 |

The in vitro permeation of diclofenac sodium through human cadaver skin from these discs was measured as described in the Methods section. Three diffusion cells were used for each formulation. The cells were filled with 10% ethanol/90% water solution. The receiver solution was completely withdrawn and replaced with fresh ethanol/water solution at each time point. The samples taken were analyzed by an HPLC for the concentration of diclofenac sodium in the receiver solution. The cumulative amount of diclofenac sodium across human cadaver skin was calculated using the measured diclofenac sodium concentrations in the receiver solutions.

TABLE 52
Cumulative Amount of Diclofenac Sodium (μg/cm²)

| Time | Diclo-1 | Diclo-2 | Diclo-3 | Diclo-4 |
|---|---|---|---|---|
| 5 hours | 0.5 | 659.0 | 1437.8 | 2010.5 |
| 10.5 hours | 4.7 | 1587.6 | 2619.3 | 2992.9 |
| 20 hours | 18.8 | 2273.7 | 3263.0 | 3513.1 |
| 24 hours | 28.4 | 2439.6 | 3420.6 | 3647.3 |

The cumulative amount of diclofenac sodium across human cadaver skin at 24 hours increased from 28.4 μg/cm² to 3647.3 μg/cm² when the calculated excess NaOH concentration in the dried patch was increased from 0% to 3.6%. The cumulative amount of diclofenac sodium across human cadaver skin at 24 hours from the system containing 1.3% NaOH (Diclo-2) was 2439.6 μg/cm², which was about 85 times higher than that from the formulation without NaOH (28.4 μg/cm², Diclo-1).

The formulation of Diclo-2 provided up to 86-fold more diclofenac sodium flux than in the absence of NaOH (Diclo-1). The formulation of Diclo-3 provided up to 120-fold more flux, while the highest pH formulation evaluated, Diclo-4, provided up to 128-fold more flux than in the absence of NaOH.

### Example 17

An in-vitro skin permeation study was conducted using four diclofenac sodium transdermal gels, designated Diclo-5, Diclo-6, Diclo-7, and Diclo-8, the compositions of which are set forth in Table 53.

TABLE 53
Component Weight and Weight Percent Based on Total Solution
Weight

| | Diclo-5 | Diclo-6 | Diclo-7 | Diclo-8 |
|---|---|---|---|---|
| | g (wt %) | g wt %) | g (wt %) | g (wt %) |
| Diclofenac | 0.3 (14.1) | 0.3 (13.8) | 0.3 (13.7) | 0.3 (13.50) |
| sodium | | | | |
| PG | 0.6 (28.2) | 0.6 (27.6) | 0.6 (27.4) | 0.6 (26.9) |
| Ethyl alcohol | 1 (46.9) | 1 (46.1) | 1 (45.7) | 1 (44.8) |
| DI water | 0.2 (9.4) | 0.22 (10.1) | 0.23 (10.5) | 0.25 (11.2) |
| HPMC | 0.03 (1.4) | 0.03 (1.4) | 00.3 (1.4) | 0.03 (1.3) |
| NaOH | 0 | 0.02 (0.9) | 0.03 (1.4) | 0.05 (2.2) |

Since diclofenac sodium is not expected to react with NaOH, the NaOH concentration listed in Table 53 equals the excess NaOH concentration, calculated as described in Example 2.

The in vitro permeation of diclofenac sodium through human cadaver skin from these gels was measured as described in Example 6. Three diffusion cells were used for each formulation. 10% ethanol/90% water solution was used as the receiver solution. The volume of receiver solution was 8 ml. The receiver solution was collected and replaced with fresh ethanol/water solution at each time point. The receiver solution collected was analyzed by an HPLC for the concentration of diclofenac sodium. The cumulative amount of diclofenac sodium across human cadaver skin was calculated using the measured diclofenac sodium concentrations in the receiver solutions.

TABLE 54
Cumulative Amount of Diclofenac Sodium (μg/cm²)

| Time | Diclo-5 | Diclo-6 | Diclo-7 | Diclo-8 |
|---|---|---|---|---|
| 5 hours | 16.8 | 50.6 | 175.9 | 585.2 |
| 10.5 hours | 29.8 | 147.5 | 503.5 | 1499.8 |
| 20 hours | 53.4 | 252.3 | 896.4 | 1988.1 |
| 24 hours | 65.3 | 270.4 | 1023.3 | 2036.8 |

TABLE 55
Excess NaOH Concentration (wt %)

| | Diclo-5 | Diclo-6 | Diclo-7 | Diclo-8 |
|---|---|---|---|---|
| Excess NaOH | 0 | 0.9 | 1.4 | 2.2 |
| Concentration | | | | |

The cumulative amount of diclofenac sodium across human cadaver skin at 24 hours increased from 65.3 μg/cm² to 2036.8 μg/cm² when the calculated excess NaOH concentration in the gel was increased from 0% to 2.2%. The cumulative amount of diclofenac sodium across human cadaver skin at 24 hours from the gel containing 0.2% NaOH (Diclo-6) was 270.4 μg/cm², which was about 4 times higher than that from the formulation without NaOH (65.3 μg/cm², Diclo-5).

The formulation of Diclo-6 provided up to 4-fold more diclofenac sodium flux than in the absence of NaOH (Diclo-5). The formulation of Diclo-7 provided up to 16-fold more flux, while the highest pH formulation evaluated, Diclo-8, provided up to 31-fold more flux than in the absence of NaOH.

### Example 18

An in-vitro skin permeation study was conducted using four testosterone transdermal systems, designated Test-1, Test-2, Test-3, and Test-4, the compositions of which are set forth in Table 56. Round disc samples were prepared as described in the Methods section. The theoretical percent weight for each ingredient after drying (calculated assuming all the volatile ingredients were completely removed during drying) is listed in Table 57.

TABLE 56
Component Weight and Weight Percent Based on Total Solution
Weight

| | Test-1 | Test-2 | Test-3 | Test-4 |
|---|---|---|---|---|
| | g (wt %) | g (wt %) | g (wt %) | g (wt %) |
| Testosterone | 0.3 (4.8) | 0.3 (4.7) | 0.3 (4.7) | 0.3 (4.7) |
| Ethyl alcohol | 0.5 (7.9) | 0.5 (7.9) | 0.5 (7.8) | 0.5 (7.8) |
| PG | 0.5 (7.9) | 0.5 (7.9) | 0.5 (7.8) | 0.5 (7.8) |
| NaOH | 0 | 0.02 (0.3) | 0.04 (0.6) | 0.075 (1.2) |
| DI water | 0 | 0.02 (0.3) | 0.04 (0.6) | 0.075 (1.2) |
| PIB adhesive | 4 (63.5) | 4 (63.1) | 4 (62.7) | 4 (62.0) |
| (30% solid) | | | | |
| Heptane | 1 (15.9) | 1 (15.8) | 1 (15.7) | 1 (15.5) |

TABLE 57
Component Weight and Weight Percent Based on Dried Film Weight

| | Test-1 | Test-2 | Test-3 | Test-4 |
|---|---|---|---|---|
| | g (wt %) | g (wt %) | g (wt %) | g (wt %) |
| Testosterone | 0.3 (15.0) | 0.3 (14.9) | 0.3 (14.7) | 0.3 (14.5) |
| PG | 0.5 (25.0) | 0.5 (24.8) | 0.5 (24.5) | 0.5 (24.1) |
| NaOH | 0 | 0.02 (1.0) | 0.04 (2.0) | 0.075 (3.6) |
| PIB adhesive | 1.2 (60.0) | 1.2 (59.4) | 1.2 (58.8) | 1.2 (57.8) |
| (30% solid) | | | | |

Since testosterone is not expected to react with NaOH, the NaOH concentration listed in Table 57 equals the excess NaOH concentration, calculated as described in Example 2.

The pH of the patches was measured as described in the Methods section. The pH of the testosterone patch increased from 7.14 to 10.32 when the calculated excess NaOH concentration in the dried patch was increased from 0% to 3.6%.

TABLE 58
Excess NaOH Concentration (wt %) and pH

| | Test-1 | Test-2 | Test-3 | Test-4 |
|---|---|---|---|---|
| Excess NaOH | 0 | 1.0 | 2.0 | 3.6 |
| Concentration | | | | |
| pH | 7.14 | 9.17 | 10.04 | 10.32 |

The in vitro permeation of testosterone through human cadaver skin from these discs was measured as described in the Methods section. Three diffusion cells were used for each formulation. The cells were filled with 10% ethanol/90% water solution. The receiver solution was completely withdrawn and replaced with fresh ethanol/water solution at each time point. The samples taken were analyzed by an HPLC for the concentration of testosterone in the receiver solution. The cumulative amount of testosterone across human cadaver skin was calculated using the measured testosterone concentrations in the receiver solutions.

TABLE 59
Cumulative Amount of Testosterone (μg/cm²)

| Time | Test-1 | Test-2 | Test-3 | Test-4 |
|---|---|---|---|---|
| 5 hours | 1.9 | 7.3 | 36.1 | 76.1 |
| 16.25 hours | 4.3 | 28.5 | 78.0 | 147.8 |
| 20 hours | 5.3 | 36.6 | 89.5 | 168.8 |
| 24 hours | 7.4 | 49.9 | 108.0 | 199.4 |

The cumulative amount of testosterone across human cadaver skin at 24 hours increased from 7.4 μg/cm² to 199.4 μg/cm² when the calculated excess NaOH concentration in the dried patch was increased from 0% to 3.6%. The cumulative amount of testosterone across human cadaver skin at 24 hours from the system containing 1.0% NaOH (Test-2) was 49.9 μg/cm², which was about six times higher than that from the formulation without NaOH (7.4 μg/cm², Test-1). This result indicated that the permeation of testosterone could be enhanced with an excess NaOH concentration as low as 1.0%.

The formulation of Test-P92 provided up to 7-fold more testosterone flux than in the absence of NaOH (Test-1). The formulation of Test-3 provided up to 19-fold more flux, while the highest pH formulation evaluated, Test-4, provided up to 40-fold more flux than in the absence of NaOH.

### Example 19

An in-vitro skin permeation study was conducted using four diclofenac sodium transdermal systems, designated Diclo-9, Diclo-10, Diclo-11, and Diclo-12, the compositions of which are set forth in Table 60. Round disc samples were prepared as described in the Methods section. The theoretical percent weight for each ingredient after drying (calculated assuming all the volatile ingredients were completely removed during drying) is listed in Table 61.

TABLE 60
Component Weight and Weight Percent
Based on Total Solution Weight

| | Diclo-9 | Diclo-10 | Diclo-11 | Diclo-12 |
|---|---|---|---|---|
| | g (wt %) | g (wt %) | g (wt %) | g (wt %) |
| Diclofenac | 0.6 (9.2) | 0.6 (9.2) | 0.9 (9.2) | 0.6 (9.1) |
| sodium | | | | |
| PG | 0.9 (13.8) | 0.9 (13.8) | 0.9 (13.8) | 0.9 (13.6) |
| NaOH | 0 | 0.01 (0.2) | 0.02 (0.3) | 0.05 (0.8) |
| PIB adhesive | 4 (61.5) | 4 (61.3) | 4 (61.2) | 4 (60.6) |
| (30% solid) | | | | |
| Heptane | 1 (15.4) | 1 (15.3) | 1 (15.3) | 1 (15.2) |
| DI water | 0 | 0.01 (0.2) | 0.02 (0.3) | 0.05 (0.8) |

TABLE 61
Component Weight and Weight Percent
Based on Dried Film Weight

| | | | |
|---|---|---|---|
| Diclo-9 | Diclo-10 | Diclo-11 | Diclo-12 |
| g (wt %) | g (wt %) | g (wt %) | g (wt %) |

| | | | | |
|---|---|---|---|---|
| Diclofenac | 0.6 (22.2) | 0.6 (22.1) | 0.9 (22.1) | 0.6 (21.8) |
| sodium | | | | |
| PG | 0.9 (33.3) | 0.9 (33.2) | 0.9 (33.1) | 0.9 (32.7) |
| NaOH | 0 | 0.01 (0.4) | 0.02 (0.7) | 0.05 (1.8) |
| PIB adhesive | 1.2 (44.4) | 1.2 (44.3) | 1.2 (44.1) | 1.2 (43.6) |
| (30% solid) | | | | |

Since diclofenac sodium is not expected to react with NaOH, the NaOH concentration listed in Table 61 equals the excess NaOH concentration, calculated as described in Example 2.

The pHs of the receiver solutions at various time points are listed below.

TABLE 62
pH of Receiver Solutions

| Time | Diclo-9 | Diclo-10 | Diclo-11 | Diclo-12 |
|---|---|---|---|---|
| 3 hours | 8.1 | 8.0 | 9.3 | 10.8 |
| 6 hours | 7.4 | 7.9 | 7.7 | 10.0 |
| 10 hours | 7.0 | 7.6 | 7.3 | 7.7 |
| 24 hours | 7.0 | 8.9 | 7.5 | 9.6 |

The pH of the patches was measured as described in the Methods section. The pH of the diclofenac sodium patch increased from 7.40 to 10.38 when the calculated excess NaOH concentration in the dried patch was increased from 0% to 1.8%.

TABLE 63
Excess NaOH Concentration (wt %) and pH

| | Diclo-9 | Diclo-10 | Diclo-11 | Diclo-12 |
|---|---|---|---|---|
| Excess NaOH | 0 | 0.4 | 0.7 | 1.8 |
| Concentration | | | | |
| pH | 7.40 | 8.99 | 10.71 | 10.38 |

The in vitro permeation of diclofenac sodium through human cadaver skin from these discs was measured as described in the Methods section. Twelve diffusion cells were used for each formulation. The cells were filled with 10% ethanol/90% water solution. At each time point, the pH at the interface between skin and the patch for three diffusion cells was measured by removing the receiving fluid, removing the clamp and the donor chamber, gently teasing the patch away from the skin with tweezers, leaving the skin on the receiver chamber, measuring the pH of the solution on the skin by placing the microelectrode directly onto the skin surface.

The cumulative amount of diclofenac sodium across human cadaver skin was calculated using the measured diclofenac sodium concentrations in the receiver solutions.

TABLE 64
Cumulative Amount of Diclofenac Sodium (μg/cm²)

| Time | Diclo-9 | Diclo-10 | Diclo-11 | Diclo-12 |
|---|---|---|---|---|
| 3 hours | 7.5 | 1.5 | 33.4 | 257.7 |
| 6 hours | 39.6 | 18.3 | 269.3 | 793.3 |
| 10 hours | 63.2 | 49.3 | 654.4 | 1652.2 |
| 24 hours | 34.6 | 227.7 | 1733.8 | 3257.7 |

The cumulative amount of diclofenac sodium across human cadaver skin at 24 hours increased from 34.6 μg/cm² to 3257.7 μg/cm² when the calculated excess NaOH concentration in the dried patch was increased from 0% to 1.8%. The cumulative amount of diclofenac sodium across human cadaver skin at 24 hours from the system containing 0.4% NaOH (Diclo-10) was 227.7 μg/cm², which was about six times higher than that from the formulation without NaOH (34.6 μg/cm², Diclo-9). This result indicated that the permeation of diclofenac sodium across human skin could be enhanced by a NaOH concentration as low as 0.4%.

The formulation of Diclo-10 provided up to 7-fold more diclofenac sodium flux than in the absence of NaOH (Diclo-9). The formulation of Diclo-11 provided up to 50-fold more flux, while the highest pH formulation evaluated, Diclo-12, provided up to 94-fold more flux than in the absence of NaOH.

The measured pHs at the skin/patch interface are listed below.

TABLE 65
pHs at the Interface between Skin and Patch

| Time | Diclo-9 | Diclo-10 | Diclo-11 | Diclo-12 |
|---|---|---|---|---|
| 3 hours | * | 11.0 | * | 10.3 |
| 6 hours | * | 11.0 | 11.2 | 9.8 |
| 10 hours | 8.5 | 10.9 | 10.7 | 10.2 |
| 24 hours | * | 9.7 | 10.1 | 9.4 |

* Could not be measured because there was not enough solution at the interface.

The pHs at the interface between skin and the patch remained about the same, even though the concentration of NaOH was increased from 0.4% to 1.8%. It was difficult to measure the pH of interface between skin and patch for the formulations without NaOH or with a low NaOH concentration because there was not enough solution on the top of the skin.

Since the pH measurement for the interface between the skin and patch may be difficult for low NaOH concentrations, the pHs of the receiver solutions were measured at various time points as references. The pHs of receiver solutions indicated that the pHs depend on the time interval between sampling, the NaOH concentration in the patch and the time point. The pHs at the 3-hour time point increased from 8.0 to 10.8 when the NaOH concentration in the patch was increased from 0.4% to 1.8%.

### Example 20

An in-vitro skin permeation study was conducted using three alendronate sodium transdermal systems, designated, Al-1, Al-2 and Al-3, the compositions of which are set forth in Table 66.

Round disc samples were prepared in a manner similar to that described in the Methods section, except that the formulation was dried at a temperature of 65° C. and the discs were cut into discs having a diameter of 9/16 inch.

The theoretical percent weight for each ingredient after drying (calculated assuming all the volatile ingredients were completely removed during drying) is listed in Table 67.

TABLE 66
Component Weight and Weight Percent
Based on Total Solution Weight

| | A1-1 | A1-2 | A1-3 |
|---|---|---|---|
| | g (wt %) | g (wt %) | g (wt %) |
| Alendronate sodium | 0.30 (3.2) | 0.30 (3.2) | 0.30 (3.2) |
| Glycerin | 1.00 (10.8) | 1.00 (10.6) | 1.00 (10.5) |
| NaOH | 0 | 0.05 (0.5) | 0.10 (1.1) |
| PIB adhesive | 7.5 (80.6) | 7.5 (79.8) | 7.5 (78.9) |
| (30% solid) | | | |
| Heptane | 0.50 (5.4) | 0.50 (5.3) | 0.50 (5.3) |
| DI water | 0 | 0.05 (0.5) | 0.10 (1.1) |

TABLE 67
Component Weight and Weight Percent
Based on Dried Film Weight

| | A1-1 | A1-2 | A1-3 |
|---|---|---|---|
| | g (wt %) | g (wt %) | g (wt %) |
| Alendronate sodium | 0.30 (8.5) | 0.30 (8.3) | 0.30 (8.2) |
| Glycerin | 1.00 (28.2) | 1.00 (27.8) | 1.00 (27.4) |
| NaOH | 0 | 0.05 (1.4) | 0.10 (2.7) |
| PIB adhesive | 2.25 (63.4) | 2.25 (62.5) | 2.25 (61.6) |

Even though alendronate sodium may behave as an acid and react with NaOH, the amount of NaOH consumed by this reaction was not determined. For the ease of comparison, it was assumed that the reaction between alendronate sodium and NaOH was not significant. Therefore, the NaOH concentration listed in Table 67 equals the excess NaOH concentration, calculated as described in Example 2.

The pH of the patches was measured as described in the Methods section but using a 2.4 cm² circular patch. The pH of the alendronate sodium patch increased from 5.50 to 9.66 when the calculated excess NaOH concentration in the dried patch was increased from 0% to 2.7%.

TABLE 68
Excess NaOH Concentration (wt %) and pH

| | Al-1 | Al-2 | Al-3 |
|---|---|---|---|
| Excess NaOH | 0 | 1.4% | 2.7% |
| Concentration | | | |
| pH | 5.50 | 6.66 | 9.66 |

The in vitro permeation of alendronate sodium through human cadaver skin from these discs was measured as described in the Methods section. Three diffusion cells were used for each formulation. The receiver solution, PBS buffer (0.05 M KH₂PO₄ with 0.15 M NaCl, pH adjusted to 6.5), was completely withdrawn and replaced with fresh receiver solution at each time point. The samples taken were analyzed by a derivatization method for the concentration of alendronate sodium in the receiver solution. The cumulative amount of alendronate sodium across human cadaver skin was calculated using the measured alendronate sodium concentrations in the receiver solutions.

TABLE 69
Cumulative Amount of Alendronate Sodium (mg/cm²)

| Time | Al-1 | Al-2 | Al-3 |
|---|---|---|---|
| 5.5 hours | 0.046 | 0.303 | 0.466 |
| 18 hours | 0.215 | 0.498 | 0.784 |
| 24 hours | 0.301 | 0.555 | 0.873 |

The cumulative amount of alendronate sodium across human cadaver skin at 24 hours increased from 0.301 mg/cm² to 0.873 mg/cm² when the calculated excess NaOH concentration in the dried patch was increased from 0% to 2.7%.

The formulation of Al-2 provided up to 2-fold more alendronate sodium flux than in the absence of NaOH (Al-1). The highest pH formulation evaluated, Al-3, provided up to 3-fold more flux than in the absence of NaOH.

### Example 21

An in-vitro skin permeation study was conducted using three risperidone transdermal systems, designated, Rispe-1, Rispe-2 and Rispe-3, the compositions of which are set forth in Table 70.

Round disc samples were prepared in a manner similar to that described in the Methods section, except that the formulation was dried at a temperature of 65° C. and the discs were cut into discs having a diameter of 9/16 inch.

The theoretical percent weight for each ingredient after drying (calculated assuming all the volatile ingredients were completely removed during drying) is listed in Table 71.

TABLE 70
Component Weight and Weight Percent Based on Total Solution
Weight

| | Rispe-1 | Rispe-2 | Rispe-3 |
|---|---|---|---|
| | g (wt %) | g (wt %) | g (wt %) |
| Risperidone | 0.30 (3.4) | 0.30 (3.3) | 0.30 (3.3) |
| Benzyl Alcohol | 0.40 (4.5) | 0.40 (4.5) | 0.40 (4.4) |
| Tetraglycol | 1.20 (13.5) | 1.20 (13.4) | 1.20 (13.3) |
| PIB adhesive | 7.00 (78.7) | 7.00 (78.0) | 7.00 (77.4) |
| (30% solid) | | | |
| NaOH | 0 | 0.04 (0.4) | 0.07 (0.8) |
| DI water | 0 | 0.04 (0.4) | 0.07 (0.8) |

TABLE 71
Component Weight and Weight Percent Based on Dried Film Weight

| | Rispe-1 | Rispe-2 | Rispe-3 |
|---|---|---|---|
| | g (wt %) | g (wt %) | g (wt %) |
| Risperidone | 0.30 (7.5) | 0.30 (7.4) | 0.30 (7.4) |
| Benzyl Alcohol | 0.40 (10.0) | 0.40 (9.9) | 0.40 (9.8) |
| Tetraglycol | 1.20 (30.0) | 1.20 (29.7) | 1.20 (29.5) |
| PIB adhesive | 2.10 (52.5) | 2.10 (52.0) | 2.10 (51.6) |
| NaOH | 0 | 0.04 (0.9) | 0.07 (1.7) |

Since the reaction between risperidone and NaOH is not expected to be significant, the concentration of NaOH in the system is assumed to be independent from the amount of risperidone added. Therefore, the NaOH concentration listed in Table 71 equals the excess NaOH concentration, defined as described in Example 2.

The pH of the patches was measured as described in the Methods section, but using a 2.4 cm² circular patch. The pH of the risperidone patch measured increased from 7.98 to 10.15 when the calculated excess NaOH concentration in the dried patch was increased from 0% to 1.7%.

TABLE 72
Excess NaOH Concentration (wt %) and pH

| | Rispe-1 | Rispe-2 | Rispe-3 |
|---|---|---|---|
| Excess NaOH | 0 | 0.9% | 1.7% |
| Concentration | | | |
| pH | 7.98 | 8.79 | 10.15 |

The in vitro permeation of risperidone through human cadaver skin from these discs was measured as described in the Methods section. Three diffusion cells were used for each formulation. The receiver solution, 5% ethanol/95% PBS buffer (0.05 M KH₂PO₄with 0.15 M NaCl, pH adjusted to 6.5), was completely withdrawn and replaced with fresh receiver solution at each time point. The samples taken were analyzed by an HPLC for the concentration of risperidone in the receiver solution. The cumulative amount of risperidone across human cadaver skin was calculated using the measured risperidone concentrations in the receiver solutions.

TABLE 73
Cumulative Amount of Risperidone (mg/cm²)

| Time | Rispe-1 | Rispe-2 | Rispe-3 |
|---|---|---|---|
| 5 hours | 0 | 0.024 | 0.092 |
| 17.75 hours | 0.004 | 0.092 | 0.264 |
| 24 hours | 0.009 | 0.132 | 0.312 |

The cumulative amount of risperidone across human cadaver skin at 24 hours increased from 0.009 mg/cm² to 0.312 mg/cm² when the calculated excess NaOH concentration in the dried patch was increased from 0% to 1.7%.

The formulation of Rispe-2 provided up to 15-fold more risperidone flux than in the absence of NaOH (Rispe-1). The highest pH formulation evaluated, Rispe-3, provided up to 35-fold more flux than in the absence of NaOH.

### Example 22

An in vitro skin permeation study was conducted using three paroxetine hydrochloride transdermal systems, designated Pax-1, Pax-2 and Pax-3, the compositions of which are set forth in Table 74.

Round disc samples were prepared in a manner similar to that described in the Methods section, except that the formulation was dried at a temperature of 65° C. and the discs were cut into discs having a diameter of 9/16 inch.

The theoretical percent weight for each ingredient after drying (calculated assuming all volatile ingredients were completely removed during drying) is set forth in Table 75.

TABLE 74
Component Weight and Weight Percent Based on Total Solution
Weight

| | Pax-1 | Pax-2 | Pax-3 |
|---|---|---|---|
| | g (wt %) | g (wt %) | g (wt %) |
| Paroxetine HCl | 0.30 (5.1) | 0.30 (5.0) | 0.30 (4.9) |
| DI Water | 0.30(5.1) | 0.35 (5.8) | 0.40 (6.6) |
| THF | 0.20 (3.4) | 0.20 (3.3) | 0.20 (3.3) |
| NaGH | 0 | 0.05 (0.8) | 0.10 (1.6) |
| Benzyl Alcohol | 0.30 (5.1) | 0.30 (5.0) | 0.30 (4.9) |
| Glycerin | 0.30 (5.1) | 0.30 (5.0) | 0.30 (4.9) |
| PIB adhesive | 4.00 (67.8) | 4.00 (66.7) | 4.00 (65.6) |
| (30% solid) | | | |
| n-Heptane | 0.50 (8.5) | 0.50 (8.3) | 0.50 (8.2) |

TABLE 75
Weight and Theoretical Weight Percent Based on Dried Film Weight

| | Pax-1 | Pax-2 | Pax-3 |
|---|---|---|---|
| | g (wt %) | g (wt %) | g (wt %) |
| Paroxetine HCl | 0.30 (14.3) | 0.30 (14.0) | 0.30 (13.6) |
| NaOH | 0 | 0.05 (2.3) | 0.10 (4.5) |
| Benzyl Alcohol | 0.30 (14.3) | 0.30 (14.0) | 0.30 (13.6) |
| Glycerin | 0.30 (14.3) | 0.30 (14.0) | 0.30 (13.6) |
| PIB adhesive | 1.20 (57.1) | 1.20 (55.8) | 1.20 (54.5) |

Since paroxetine HCl is an acid addition salt of a free base, it reacts with NaOH. The concentration of NaOH in the system after the reaction is completed depends on the amount of paroxetine HCl added. The remaining NaOH concentration after the reaction is completed is defined as the excess NaOH concentration, and was calculated as described in Example 2. The pH was measured as described in the Methods section but using a 2.4 cm² circular patch. The pH of the paroxetine HCl patch increased from 9.32 to 10.62 when the calculated excess NaOH concentration in the dried patch was increased from 0.8% to 3.1%. The pH of the patch without NaOH was 7.37.

TABLE 76
Excess NaOH Concentration (wt %) and pH

| | Pax-1 | Pax-2 | Pax-3 |
|---|---|---|---|
| Excess NaOH | — | 0.8% | 3.1% |
| Concentration | | | |
| pH | 7.37 | 9.32 | 10.62 |

The in vitro permeation of paroxetine HCl through human cadaver skin from these discs was measured as described in the Methods section. Three diffusion cells were used for each formulation. The receiver solution, 5% N-methylpyrrolidone/95% water, was completely withdrawn and replaced with fresh receiver solution at each time point. The samples taken were analyzed by an HPLC for the concentration of paroxetine HCl in the receiver solution. The cumulative amount of paroxetine HCl that permeated across the human cadaver skin was calculated using the measured paroxetine HCl concentrations in the receiver solutions.

TABLE 77
Cumulative Amount of paroxetine HCl (mg/cm²)

| Time | Pax-1 | Pax-2 | Pax-3 |
|---|---|---|---|
| 4.75 hours | 0.014 | 0.008 | 0.145 |
| 17.75 hours | 0.082 | 0.141 | 0.616 |
| 24 hours | 0.133 | 0.247 | 0.850 |

The cumulative amount of paroxetine HCl across human cadaver skin at 24 hours increased from 0.247 mg/cm² to 0.850 mg/cm² when the calculated excess NaOH concentration in the dried patch was increased from 0.8% to 3.1% as compared to 0.133 mg/cm² for the formulation without NaOH.

The formulation of Pax-2 provided up to 2-fold more paroxetine HCl flux than in the absence of NaOH (Pax-1). The highest pH formulation evaluated, Pax-3, provided up to 6-fold more flux than in the absence of NaOH.

### Example 23

An in vitro skin permeation study was conducted using three galanthamine hydrobromide transdermal systems, designated Gala-1, Gala-2 and Gala-3, the compositions of which are set forth in Table 78.

Round disc samples were prepared in a manner similar to that described in the Methods section, except that the formulation was dried at a temperature of 65° C. and the discs were cut into discs having a diameter of 9/16 inch.

The theoretical percent weight for each ingredient after drying (calculated assuming all volatile ingredients were completely removed during drying) is set forth in Table 79.

TABLE 78
Component Weight and Weight Percent Based on Total Solution
Weight

| | Gala-1 | Gala-2 | Gala-3 |
|---|---|---|---|
| | g (wt %) | g (wt %) | g (wt %) |
| Galanthamine HBr | 0.40 (4.7) | 0.40 (4.6) | 0.40 (4.6) |
| DI Water | 0.30 (3.5) | 0.34 (3.9) | 0.38 (4.3) |
| NaOH | 0 | 0.04 (0.5) | 0.08 (0.9) |
| Glycerin | 1.00 (11.6) | 1.00 (11.5) | 1.00 (11.4) |
| Benzyl Alcohol | 0.40 (4.7) | 0.40 (4.6) | 0.40 (4.6) |
| PIB adhesive | 6.00 (69.8) | 6.00 (69.1) | 6.00 (68.5) |
| (30% solid) | | | |
| n-Heptane | 0.50 (5.8) | 0.50 (5.8) | 0.50 (5.7) |

TABLE 79
Weight and Theoretical Weight Percent Based on Dried Film Weight

| | Gala-1 | Gala-2 | Gala-3 |
|---|---|---|---|
| | g (wt %) | g (wt %) | g (wt %) |
| Galanthamine HBr | 0.40 (11.1) | 0.40 (11.0) | 0.40 (10.9) |
| NaOH | 0 | 0.04 (1.1) | 0.08 (2.2) |
| Glycerin | 1.00 (27.8) | 1.00 (27.5) | 1.00 (27.2) |
| Benzyl Alcohol | 0.40 (11.1) | 0.40 (11.0) | 0.40 (10.9) |
| PIB adhesive | 1.80 (50.0) | 1.80 (49.5) | 1.80 (48.9) |

Since galanthamine HBr is an acid addition salt of a free base, it reacts with NaOH. The concentration of NaOH in the system after the reaction is completed depends on the amount of galanthamine HBr added. The remaining NaOH concentration after the reaction is completed is defined as the excess NaOH concentration, and was calculated as described in Example 2. The pH was measured as described in the Methods section but using a 2.4 cm² circular patch. The pH of the galanthamine HBr patch increased from 8.73 to 10.56 when the calculated excess NaOH concentration in the dried patch was increased from 0% to 1.0%. The pH of the formulation without NaOH was 6.53.

TABLE 80
Excess NaOH Concentration (wt %) and pH

| | Gala-1 | Gala-2 | Gala-3 |
|---|---|---|---|
| Excess NaOH | — | 0% | 1.0% |
| Concentration | | | |
| pH | 6.53 | 8.73 | 10.56 |

The in vitro permeation of galanthamine HBr through human cadaver skin from these discs was measured as described in the Methods. Three diffusion cells were used for each formulation. The receiver solution, 5% ethanol/95% PBS buffer (0.05 M KH₂PO₄ with 0.15 M NaCl, pH adjusted to 6.5), was completely withdrawn and replaced with fresh receiver solution at each time point. The samples taken were analyzed by an HPLC for the concentration galanthamine HBr in the receiver solution. The cumulative amount of galanthamine HBr that permeated across the human cadaver skin was calculated using the measured galanthamine HBr concentrations in the receiver solutions.

TABLE 81
Cumulative Amount of Galanthamine HBr (mg/cm²)

| Time | Gala-1 | Gala-2 | Gala-3 |
|---|---|---|---|
| 6 hours | 0.140 | 0.480 | 0.475 |
| 18 hours | 0.429 | 1.058 | 1.412 |
| 24 hours | 0.624 | 1.254 | 1.750 |

The cumulative amount of galanthamine HBr across human cadaver skin at 24 hours increased from 1.254 mg/cm² to 1.750 mg/cm² when the calculated excess NaOH concentration in the dried patch was increased from 0% to 1.0% as compared to 0.624 mg/cm² for the formulation without NaOH.

The formulation of Gala-2 provided up to 2-fold more galanthamine HBr flux than in the absence of NaOH (Gala-1). The highest pH formulation evaluated, Gala-3, provided up to 3-fold more flux than in the absence of NaOH.

### Example 24

An in vitro skin permeation study was conducted using three hydromorphone hydrochloride transdermal systems, designated Hymo-1, Hymo-2 and Hymo-3, the compositions of which are set forth in Table 82.

Round disc samples were prepared as described in the Methods section, except that the formulation was dried at a temperature of 65° C. and the discs were cut into discs having a diameter of 9/16 inch.

The theoretical percent weight for each ingredient after drying (calculated assuming all volatile ingredients were completely removed during drying) is set forth in Table 83.

TABLE 82
Component Weight and Weight Percent Based on Total Solution
Weight

| | Hymo-1 | Hymo-2 | Hymo-3 |
|---|---|---|---|
| | g (wt %) | g (wt %) | g (wt %) |
| Hydromorphone HCl | 0.20 (2.8) | 0.20 (2.7) | 0.20 (2.7) |
| DI Water | 0.30 (4.1) | 0.38 (5.1) | 0.43 (5.7) |
| NaOH | 0 | 0.08 (1.0) | 0.13 (1.7) |
| Glycerin | 1.25 (17.2) | 1.25 (16.9) | 1.25 (16.7) |
| PIB adhesive | 5.00 (69.0) | 5.00 (67.6) | 4.00 (66.7) |
| (30% solid) | | | |
| n-Heptane | 0.50 (6.9) | 0.50 (6.8) | 0.50 (6.7) |

TABLE 83
Weight and Theoretical Weight Percent Based on Dried Film Weight

| | Hymo-1 | Hymo-2 | Hymo-3 |
|---|---|---|---|
| | g (wt %) | g (wt %) | g (wt %) |
| Hydromorphone HCl | 0.20 (6.8) | 0.20 (6.6) | 0.20 (6.5) |
| NaOH | 0 | 0.08 (2.5) | 0.13 (4.1) |
| Glycerin | 1.25 (42.4) | 1.25 (41.3) | 1.25 (41.7) |
| PIB adhesive | 1.50 (50.8) | 1.50 (49.6) | 1.50 (48.8) |

The in vitro permeation of hydromorphone HCl through human cadaver skin from these discs was measured as described in the Methods section. Three diffusion cells were used for each formulation. The receiver solution, 5% ethanol in 0.05 M KH₂PO₄, was completely withdrawn and replaced with fresh receiver solutions at each time point. The samples taken were analyzed by an HPLC for the concentration of hydromorphone HCl in the receiver solution. The cumulative amount of hydromorphone HCl that permeated across the human cadaver skin was calculated using the measured hydromorphone HCl concentrations in the receiver solutions.

TABLE 84
Cumulative Amount of Hydromorphone HCl (mg/cm²)

| Time | Hymo-1 | Hymo-2 | Hymo-3 |
|---|---|---|---|
| 5.25 hours | 0.023 | 0.076 | 0.163 |
| 17.5 hours | 0.056 | 0.185 | 0.378 |
| 24 hours | 0.076 | 0.252 | 0.476 |

Since hydromorphone HCl is an acid addition salt of a free base, it reacts with NaOH. The concentration of NaOH in the system after the reaction is completed depends on the amount of hydromorphone HCl added. The remaining NaOH concentration after the reaction is completed is defined as the excess NaOH concentration, and was calculated as described in Example 2. The pH was measured as described in the Methods section but using a 2.4 cm² circular patch.

TABLE 85
Excess NaOH Concentration (wt %) and pH

| | Hymo-1 | Hymo-2 | Hymo-3 |
|---|---|---|---|
| Excess NaOH | — | 1.7% | 3.3% |
| Concentration | | | |
| pH | 6.61 | 8.93 | 10.48 |

The pH of the hydromorphone HCl patch increased from 8.93 to 10.48 when the calculated excess NaOH concentration in the dried patch was increased from 1.7% to 3.3%. The pH of the patch without NaOH was 6.61.

The cumulative amount of hydromorphone HCl across human cadaver skin at 24 hours increased from 0.252 mg/cm² to 0.476 mg/cm² when the calculated excess NaOH concentration in the dried patch was increased from 1.7% to 3.3% as compared to 0.076 mg/cm² for the formulation without NaOH.

The formulation of Hymo-2 provided up to 3-fold more hydromorphone HCl flux than in the absence of NaOH (Hymo-1). The highest pH formulation evaluated, Hymo-3, provided up to 6-fold more flux than in the absence of NaOH.

### Example 25

An in-vitro skin permeation study was conducted using three lidocaine transdermal systems, designated, Lido-1, Lido-2, Lido-3, the compositions of which are set forth in Table 86.

Round disc samples were prepared as described in the Methods section, except that the formulation was dried at a temperature of 65° C. and the discs were cut into discs having a diameter of 9/16 inch.

The theoretical percent weight for each ingredient after drying (calculated assuming all the volatile ingredients were completely removed during drying) is listed in Table 87.

TABLE 86
Component Weight and Weight Percent Based on Total Solution
Weight

| | Lido-1 | Lido-2 | Lido-3 |
|---|---|---|---|
| | g (wt %) | g (wt %) | g (wt %) |
| Lidocaine | 0.50 (9.1) | 0.50 (8.9) | 0.50 (8.8) |
| PG | 0.50 (9.1) | 0.50 (8.9) | 0.50 (8.8) |
| Water | 0 | 0.07(1.2) | 0.11 (1.8) |
| PIB adhesive | 4.00 (72.7) | 4.00 (70.9) | 4.00 (70.1) |
| (30% solid) | | | |
| NaOH | 0 | 0.07(1.2) | 0.11 (1.8) |
| n-Heptane | 0.50 (9.1) | 0.50 (8.9) | 0.50 (8.8) |

TABLE 87
Component Weight and Weight Percent Based on Dried Film Weight

| | Lido-1 | Lido-2 | Lido-3 |
|---|---|---|---|
| | g (wt %) | g (wt %) | g (wt %) |
| Lidocaine | 0.50 (22.7) | 0.50 (22.0) | 0.50 (21.7) |
| PG | 0.50 (22.7) | 0.50 (22.0) | 0.50 (21.7) |
| PIB adhesive | 1.20 (54.4) | 1.20 (52.9) | 1.20 (52.1) |
| NaOH | 0 | 0.07 (3.1) | 0.11 (4.6) |

Since the reaction between lidocaine and NaOH is not expected to be significant, the concentration of NaOH in the system is assumed to be independent from the amount of lidocaine added. Therefore, the NaOH concentration listed in Table 87 equals the excess NaOH concentration, calculated as described in Example 2.

The in vitro permeation of lidocaine through human cadaver skin from these discs was measured as described in the Methods section. Three diffusion cells were used for each formulation. The receiver solution, 5% ethanol/95% PBS buffer (0.05 M KH₂PO₄ with 0.15 M NaCl, pH adjusted to 6.5), was completely withdrawn and replaced with fresh receiver solution at each time point. The samples taken were analyzed by an HPLC for the concentration of lidocaine in the receiver solution. The cumulative amount of lidocaine across human cadaver skin was calculated using the measured lidocaine concentrations in the receiver solutions.

TABLE 88
Cumulative Amount of Lidocaine (mg/cm²)

| Time | Lido-1 | Lido-2 | Lido-3 |
|---|---|---|---|
| 5 hours | 0.069 | 0.126 | 0.300 |
| 15.5 hours | 0.237 | 0.410 | 0.816 |
| 23.75 hours | 0.428 | 0.632 | 1.169 |

The pH of the patches was measured as described in the Methods section but using a 2.4 cm² circular patch.

TABLE 89
Excess NaOH Concentration (wt %) and pH

| | Lido-1 | Lido-2 | Lido-3 |
|---|---|---|---|
| Excess NaOH | 0 | 3.1% | 4.6% |
| Concentration | | | |
| pH | 8.86 | 10.44 | 10.87 |

The pH of the lidocaine patch measured increased from 8.86 to 10.87 when the calculated excess NaOH concentration in the dried patch was increased from 0% to 4.6%. The cumulative amount of lidocaine across human cadaver skin at 24 hours increased from 0.428 mg/cm⁻¹ to 1.169 mg/cm² when the calculated excess NaOH concentration in the dried patch was increased from 0% to 4.6%.

The formulation of Lido-2 provided up to 1.5-fold more lidocaine flux than in the absence of NaOH (Lido-1). The highest pH formulation evaluated, Lido-3, provided up to 3-fold more flux than in the absence of NaOH.

### Example 26

An in vitro skin permeation study was conducted using three enalapril maleate transdermal systems, designated Enal-1, Enal-2 and Enal-3, the compositions of which are set forth in Table 90.

Round disc samples were prepared as described in the Methods section, except that the formulation was dried at a temperature of 65° C. and the discs were cut into discs having a diameter of 9/16 inch.

The theoretical percent weight for each ingredient after drying (calculated assuming all volatile ingredients were completely removed during drying) is set forth in Table 91.

TABLE 90
Component Weight and Weight Percent Based on Total Solution
Weight

| | Enal-1 | Enal-2 | Enal-3 |
|---|---|---|---|
| | g (wt %) | g (wt %) | g (wt %) |
| Enalapril Maleate | 0.50 (8.8) | 0.50 (8.4) | 0.50 (8.1) |
| PG | 0.50 (8.8) | 0.50 (8.4) | 0.50 (8.1) |
| DI Water | 0.20 (3.5) | 0.33 (5.5) | 0.45 (7.3) |
| NaOH | 0 | 0.13 (2.1) | 0.25 (4.0) |
| PIB adhesive | 4.00 (70.2) | 4.00 (67.2) | 4.00 (64.5) |
| (30% solid) | | | |
| n-Heptane | 0.50 (8.8) | 0.50 (8.4) | 0.50 (8.1) |

TABLE 91
Weight and Theoretical Weight Percent Based on Dried Film Weight

| | Enal-1 | Enal-2 | Enal-3 |
|---|---|---|---|
| | g (wt %) | g (wt %) | g (wt %) |
| Enalapril Maleate | 0.50 (22.7) | 0.50 (21.5) | 0.50 (20.4) |
| PG | 0.50 (22.7) | 0.50 (21.5) | 0.50 (20.4) |
| NaOH | 0 | 0.13 (5.4) | 0.25 (10.2) |
| PIB adhesive | 1.20 (54.5) | 1.20 (51.6) | 1.20 (49.0) |

Since enalapril maleate is an acid addition salt of a free base, it reacts with NaOH. The concentration of NaOH in the system after the reaction is completed depends on the amount of enalapril maleate added. The remaining NaOH concentration after the reaction is completed is defined as the excess NaOH concentration, and was calculated as described in Example 2. The pH of each patch was measured as described in the Methods section but using a 2.4 cm² circular patch. The pH of the enalapril maleate patch increased from 7.29 to 10.82 when the calculated excess NaOH concentration in the dried patch was increased from 1.9% to 6.9%. The pH of the patch without NaOH was 3.12.

TABLE 92
Excess NaOH Concentration (wt %) and pH

| | Enal- 1 | Enal-2 | Enal-3 |
|---|---|---|---|
| Excess NaOH | — | 1.9% | 6.9% |
| Concentration | | | |
| pH | 3.12 | 7.29 | 10.82 |

The in vitro permeation of enalapril maleate through human cadaver skin from these discs was measured as described in the Methods section. Three diffusion cells were used for each formulation. The receiver solution, 10% ethanol, was completely withdrawn and replaced with fresh receiver solution at each time point. The samples taken were analyzed by an HPLC for the concentration of enalapril maleate in the receiver solution. The cumulative amount of enalapril maleate that permeated across the human cadaver skin was calculated using the measured enalapril maleate concentrations in the receiver solutions.

TABLE 93
Cumulative Amount of Enalapril Maleate (mg/cm²)

| | | | |
|---|---|---|---|
| Time | Enal-1 | Enal-2 | Enal-3 |

| | | | |
|---|---|---|---|
| 5.25 hours | 0 | 0.021 | 1.027 |
| 17.25 hours | 0 | 0.029 | 1.640 |
| 23.75 hours | 0 | 0.029 | 1.826 |

The cumulative amount of enalapril maleate across human cadaver skin at 24 hours increased from 0.029 mg/cm² to 1.826 mg/cm² when the calculated excess NaOH concentration in the dried patch was increased from 1.9% to 6.9% as compared to undetectable flux for the formulation without NaOH. The formulation of Enal-3 provided up to 63-fold more enalapril maleate flux than the formulation of Enal-2.

All patents, publications, and other published documents mentioned or referred to in this specification are herein incorporated by reference in their entirety.

It is to be understood that while the invention has been described in conjunction with the preferred specific embodiments hereof, the foregoing description, as well as the examples which are intended to illustrate and not limit the scope of the invention, it should be understood by those skilled in the art that various changes may be made and equivalents may be substituted without departing from the scope of the invention. Other aspects, advantages and modifications will be apparent to those skilled in the art to which the invention pertains.

Accordingly, the scope of the invention should therefore be determined with reference to the appended claims, along with the full range of equivalents to which those claims are entitled.

We claim:

1. A method for enhancing the flux of an analgesic agent through a body surface, comprising:
(a) administering the analgesic agent to a localized region of a human patient's body surface; and
(b) administering a basic permeation enhancer to the localized region, the enhancer comprising a pharmaceutically acceptable inorganic base and being present in an amount effective to provide a pH within the range of about 8.0-13.0 at the localized region of the body surface during administration of the analgesic agent and to enhance the flux of the analgesic agent through the body surface without causing damage thereto.

2. The method of claim 1 wherein the pH is within the range of about 8.5-11.5.

3. The method of claim 2 wherein the pH is within the range of about 9.5-11.5.

4. The method of claim 1 wherein the base is selected from the group consisting of ammonium hydroxide, sodium hydroxide, potassium hydroxide, calcium hydroxide, magnesium hydroxide, magnesium oxide, calcium oxide, sodium acetate, sodium borate, sodium metaborate, sodium carbonate, sodium bicarbonate, sodium phosphate, potassium carbonate, potassium bicarbonate, potassium citrate, potassium acetate, potassium phosphate, ammonium phosphate, and combinations thereof.

5. The method of claim 1 wherein the base is selected from the group consisting of inorganic hydroxides, inorganic oxides, inorganic salts of weak acids, and combinations thereof.

6. The method of claim 5 wherein the base is an inorganic hydroxide.

7. The method of claim 6 wherein the inorganic hydroxide is selected from the group consisting of ammonium hydroxide, alkali metal hydroxides, and alkaline earth metal hydroxides.

8. The method of claim 7 wherein the inorganic hydroxide is ammonium hydroxide.

9. The method of claim 7 wherein the inorganic hydroxide is an alkali metal hydroxide selected from the group consisting of sodium hydroxide and potassium hydroxide.

10. The method of claim 7 wherein the inorganic hydroxide is an alkaline earth metal hydroxide selected from the group consisting of calcium hydroxide and magnesium hydroxide.

11. The method of claim 5 wherein the base is an inorganic oxide.

12. The method of claim 11 wherein the inorganic oxide is selected from the group consisting of magnesium oxide and calcium oxide.

13. The method of claim 5 wherein the base is an inorganic salt of a weak acid.

14. The method of claim 13 wherein the inorganic salt of a weak acid is selected from the group consisting of ammonium phosphate, alkali metal salts of weak acids, and alkaline earth metal salts of weak acids.

15. The method of claim 14 wherein the inorganic salt of a weak acid is ammonium phosphate.

16. The method of claim 14 wherein the inorganic salt of a weak acid is an alkali metal salt of a weak acid selected from the group consisting of sodium acetate, sodium borate, sodium metaborate, sodium carbonate, sodium bicarbonate, sodium phosphate, potassium carbonate, potassium bicarbonate, potassium citrate, potassium acetate, and potassium phosphate.

17. The method of claim 1 wherein the body surface is skin.

18. The method of claim 1 wherein the body surface is mucosal tissue.

19. The method of claim 1 wherein the analgesic agent and basic permeation enhancer are present in a single pharmaceutical formulation.

20. The method of claim 1 wherein the analgesic agent and basic permeation enhancer are present in separate pharmaceutical formulations.

21. The method of claim 20 wherein steps (a) and (b) are done simultaneously.

22. The method of claim 20 wherein step (a) is done prior to step (b).

23. The method of claim 20 wherein step (b) is done prior to step (a).

24. The method of claim 1 wherein the analgesic agent and basic permeation enhancer are administered by applying a drug delivery device to the localized region of the patient's body surface thereby forming a body surface-delivery device interface, the device comprising the analgesic agent and basic permeation enhancer, and having an outer backing layer that serves as the outer surface of the device during use.

25. The method of claim 1 wherein the basic permeation enhancer is contained within an aqueous formulation.

26. The method of claim 25 wherein the aqueous formulation has a pH within the range of about 8.0-13.0

27. The method of claim 26 wherein the pH is within the range of about 8.5-11.5.

28. The method of claim 27 wherein the pH is within the range of about 9.5-11.5.

29. The method of claim 25 wherein the aqueous formulation is selected from the group consisting of a cream, a gel, a lotion, and a paste.

30. The method of claim 1 wherein the analgesic agent is selected from the group consisting of capsaicin, clonidine, tramadol, indomethacin, pharmaceutically acceptable derivatives thereof, and combinations thereof.

31. The method of claim 1 wherein the analgesic drug is a narcotic analgesic.

32. The method of claim 31 wherein the analgesic agent is selected from the group consisting of alfentanil, buprenorphine, butorphanol, codeine, enkephalin, fentanyl, hydrocodone, hydromorphone, levorphanol, meperidine, methadone, morphine, nicomorphine, opium, oxycodone, oxymorphone, pentazocine, propoxyphene, sufentanil, pharmaceutically acceptable derivatives thereof, and combinations thereof.

33. The method of claim 32 wherein the analgesic agent is selected from the group consisting of buprenorphine, butorphanol, fentanyl, hydrocodone, hydromorphone, levorphanol, methadone, morphine, oxycodone, oxymorphone, pharmaceutically acceptable derivatives thereof, and combinations thereof.

34. The method of claim 1 wherein the flux of the analgesic agent is enhanced by at least about 3-fold.

35. The method of claim 34 wherein the flux of the analgesic agent is enhanced by at least about 6-fold.

36. A composition for the enhanced delivery of an analgesic agent through a body surface, comprising an aqueous formulation of: (a) a therapeutically effective amount of the analgesic agent; (b) a pharmaceutically acceptable inorganic base in an amount effective to provide a pH within the range of about 8.0-13.0 at the body surface during administration of the analgesic agent and to enhance the flux of the analgesic agent through the body surface without causing damage thereto; and (c) a pharmaceutically acceptable carrier suitable for topical or transdermal drug administration, wherein the composition provides for at least about 3-fold enhanced delivery.

37. The composition of claim 36 wherein the analgesic agent is an acidic species.

38. The composition of claim 37 wherein the base is present in an amount that is the total of (a) the amount required to neutralize the acidic species plus (b) an amount equal to about 0.3-7.0 wt % of the composition.

39. The composition of claim 36 wherein the analgesic agent is a non-acidic species.

40. The composition of claim 39 wherein the base is present in an amount equal to about 0.3-7.0 wt % of the composition.

41. The composition of claim 36 comprising a cream, a gel, a lotion, or a paste.

42. The composition of claim 36 wherein the composition provides for at least about 6-fold enhanced delivery.

43. The composition of claim 36 wherein the base is selected from the group consisting of inorganic hydroxides, inorganic oxides, inorganic salts of weak acids, and combinations thereof.

44. The composition of claim 43 wherein the base is an inorganic hydroxide selected from the group consisting of ammonium hydroxide, sodium hydroxide, potassium hydroxide, calcium hydroxide and magnesium hydroxide.

45. The composition of claim 43 wherein the base is an inorganic oxide selected from the group consisting of magnesium oxide and calcium oxide.

46. The composition of claim 43 wherein the base is an inorganic salt of a weak acid selected from the group consisting of ammonium phosphate, sodium acetate, sodium borate, sodium metaborate, sodium carbonate, sodium bicarbonate, sodium phosphate, potassium carbonate, potassium bicarbonate, potassium citrate, potassium acetate, and potassium phosphate.

47. The composition of claim 36 wherein the base is effective to provide a pH within the range of about 8.5-11.5 at the localized region of the body surface during administration of the analgesic agent.

48. The composition of claim 36 wherein the analgesic agent is selected from the group consisting of capsaicin, clonidine, tramadol, indomethacin, pharmaceutically acceptable derivatives thereof, and combinations thereof.

49. The composition of claim 36 wherein the analgesic drug is a narcotic analgesic.

50. The composition of claim 49 wherein the analgesic agent is selected from the group consisting of alfentanil, buprenorphine, butorphanol, codeine, enkephalin, fentanyl, hydrocodone, hydromorphone, levorphanol, meperidine, methadone, morphine, nicomorphine, opium, oxycodone, oxymorphone, pentazocine, propoxyphene, sufentanil, pharmaceutically acceptable derivatives thereof, and combinations thereof.

51. The composition of claim 50 wherein the analgesic agent is selected from the group consisting of buprenorphine, butorphanol, fentanyl, hydrocodone, hydromorphone, levorphanol, methadone, morphine, oxycodone, oxymorphone, pharmaceutically acceptable derivatives thereof, and combinations thereof.

52. The composition of claim 36 which further comprises at least one irritation-mitigating additive.

53. A system for the enhanced topical or transdermal administration of an analgesic agent, comprising: (a) at least one drug reservoir containing the analgesic agent and a pharmaceutically acceptable inorganic base, in an amount effective to enhance the flux of the analgesic agent through the body surface without causing damage thereto; (b) a means for maintaining the system in agent and base transmitting relationship to the body surface and forming a body surface-system interface; and (c) a backing layer that serves as the outer surface of the device during use, wherein the base is effective to provide a pH within the range of about 8.0-13.0 at the body surface-system interface during administration of the analgesic agent, and wherein the system provides for at least about 3-fold enhanced delivery.

54. The system of claim 53 wherein the backing layer is occlusive.

55. The system of claim 53 wherein the drug reservoir is comprised of a polymeric adhesive.

56. The system of claim 55 wherein the polymeric adhesive serves as the means for maintaining the system in agent and base transmitting relationship to the body service.

57. The system of claim 53 wherein the drug reservoir is comprised of a hydrogel.

58. The system of claim 53 wherein the drug reservoir is comprised of a sealed pouch containing the analgesic agent and inorganic base in a liquid or semi-solid formulation.

59. The system of claim 53 wherein the analgesic agent is an acidic species.

60. The system of claim 59 wherein the base is present in an amount that is the total of (a) the amount required to neutralize the acidic species plus (b) an amount equal to about 0.3-7.0 wt % of the drug reservoir.

61. The system of claim 53 wherein the analgesic agent is a non-acidic species.

62. The system of claim 61 wherein the base is present in an amount equal to about 0.3-7.0 wt % of the drug reservoir.

63. The system of claim 53 wherein the composition provides for at least about 6-fold enhanced delivery.

64. The system of claim 53 wherein the base is selected from the group consisting of inorganic hydroxides, inorganic oxides, inorganic salts of weak acids, and combinations thereof.

65. The system of claim 64 wherein the base is an inorganic hydroxide selected from the group consisting of ammonium hydroxide, sodium hydroxide, potassium hydroxide, calcium hydroxide and magnesium hydroxide.

66. The system of claim 64 wherein the base is an inorganic oxide selected from the group consisting of magnesium oxide and calcium oxide.

67. The system of claim 64 wherein the base is an inorganic salt of a weak acid selected from the group consisting of ammonium phosphate, sodium acetate, sodium borate, sodium metaborate, sodium carbonate, sodium bicarbonate, sodium phosphate, potassium carbonate, potassium bicarbonate, potassium citrate, potassium acetate, and potassium phosphate.

68. The system of claim 53 wherein the analgesic agent is selected from the group consisting of capsaicin, clonidine, tramadol, indomethacin, pharmaceutically acceptable derivatives thereof, and combinations thereof.

69. The system of claim 53 wherein the analgesic drug is a narcotic analgesic.

70. The system of claim 69 wherein the analgesic agent is selected from the group consisting of alfentanil, buprenorphine, butorphanol, codeine, enkephalin, fentanyl, hydrocodone, hydromorphone, levorphanol, meperidine, methadone, morphine, nicomorphine, opium, oxycodone, oxymorphone, pentazocine, propoxyphene, sufentanil, pharmaceutically acceptable derivatives thereof, and combinations thereof.

71. The system of claim 70 wherein the analgesic agent is selected from the group consisting of buprenorphine, butorphanol, fentanyl, hydrocodone, hydromorphone, levorphanol, methadone, morphine, oxycodone, oxymorphone, pharmaceutically acceptable derivatives thereof, and combinations thereof.

72. The system of claim 53 which further comprises at least one irritation-mitigating additive.
